(19) United States
(12) Patent Application Publication
(10) Pub. No.: US 2016/0129011 A1
(43) Pub. Date: May 12, 2016

(54) BICYCLO 2,3-BENZODIAZEPINES AND SPIROCYCLICALLY SUBSTITUTED 2,3-BENZODIAZEPINES

(71) Applicant: BAYER PHARMA AKTIENGESELLSCHAFT, Berlin (DE)

(72) Inventors: Stephan SIEGEL, Berlin (DE); Stefan BÄURLE, Berlin (DE); Arwed CLEVE, Berlin (DE); Bernard HAENDLER, Berlin (DE); Amaury Ernesto FERNÁNDEZ-MONTALVÁN, Berlin (DE); Ursula MÖNNING, Woltersdorf (DE); Sabine KRAUSE, Berlin (DE); Pascale LEJEUNE, Berlin (DE); Matthias BUSEMANN, Berlin (DE); Joachim KUHNKE, Potsdam (DE)

(21) Appl. No.: 14/772,761

(22) PCT Filed: Feb. 17, 2014
(86) PCT No.: PCT/EP2014/052984

§ 371 Date: Dec. 1, 2015

(30) Foreign Application Priority Data
Feb. 19, 2013 (DE) 10 2013 202 678.1

(51) Int. Cl. A61K 31/551; C07D 401/10; C07D 495/10; C07D 498/08; C07D 491/107; C07D 471/08; C07D 487/10; C07D 487/08; C07D 405/10; C07D 403/10; C07D 471/10; C07D 487/04; C07D 498/10; C07D 491/08; C07D 491/048; C07D 245/06

(52) U.S. Cl. CPC A61K 31/551; C07D 245/06; C07D 401/10; C07D 495/10; C07D 498/08; C07D 491/107; C07D 471/08; C07D 487/10; C07D 487/08; C07D 405/10; C07D 403/10; C07D 471/10; C07D 487/04; C07D 498/10; C07D 491/08; C07D 491/048

(57) ABSTRACT

BET-protein-inhibitory, in particular BRD4-inhibitory bicyclo- and spirocyclically substituted 2,3-benzodiazepines of the general formula (I),

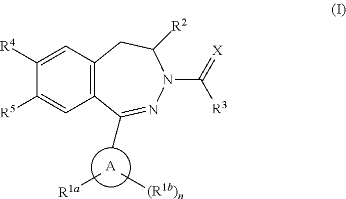

pharmaceutical compositions comprising the compounds according to the invention, and the prophylactic and therapeutic use thereof for hyperproliferative disorders, especially for tumour disorders, are described. Furthermore, the use of BET protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control is described.

The present invention relates to BET protein-inhibitory, especially BRD4-inhibitory, bicyclo- and spirocyclic substituted 2,3-benzodiazepines, to pharmaceutical compositions comprising the compounds according to the invention, and to the prophylactic and therapeutic use thereof for hyperproliferative disorders, especially for neoplastic disorders. The present invention further relates to the use of BET protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007, 282:13141-13145). The bromo domains are protein regions which recognize acetylated lysine residues. Such acetylated lysines are often found at the N-terminal end of histones (e.g. histone H3 or histone H4), and they are features of an open chromatin structure and active gene transcription (Kuo and Allis, Bioessays, 1998, 20:615-626). The different acetylation patterns recognized by BET proteins in histones were investigated in depth (Umehara et al., J. Biol. Chem., 2010, 285:7610-7618; Filippakopoulos et al., Cell, 2012, 149:214-231). In addition, bromo domains can recognize further acetylated proteins. For example, BRD4 binds to RelA, which leads to stimulation of NF-κB and transcriptional activity of inflammatory genes (Huang et al., Mol. Cell. Biol., 2009, 29:1375-1387; Zhang et al., J. Biol. Chem., 2012, doi/10.1074/jbc.M112.359505). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, suggesting a function in epigenetic memory (Dey et al., Mol. Biol. Cell, 2009, 20:4899-4909; Yang et al., Mol. Cell. Biol., 2008, 28:967-976). BRD4 is important for post-mitotic reactivation of gene transcription (Zhao et al., Nat. Cell. Biol., 2011, 13:1295-1304). It has been shown that BRD4 is essential for transcription elongation and for the recruitment of the elongation complex P-TEFb consisting of CDK9 and cyclin Ti, which leads to activation of RNA polymerase II (Yang et al., Mol. Cell, 2005, 19:535-545; Schröder et al., J. Biol. Chem., 2012, 287:1090-1099). Consequently, the expression of genes involved in cell proliferation is stimulated, for example of c-Myc and aurora B (You et al., Mol. Cell. Biol., 2009, 29:5094-5103; Zuber et al., Nature, 2011, 478:524-528). BRD2 and BRD3 bind to transcribed genes in hyperacetylated chromatin regions and promote transcription by RNA polymerase II (LeRoy et al., Mol. Cell, 2008, 30:51-60).

Knock-down of BRD4 or the inhibition of the interaction with acetylated histones in various cell lines leads to G1 arrest and to cell death apoptosis (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674).

BRD2 and BRD4 knockout mice die early in embryogenesis (Gyuris et al., Biochim. Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, doi:10.1038). Reduction in BRD4 expression leads to a selective arrest of the cell cycle and to apoptosis. Treatment with a BRD4 inhibitor prevents the proliferation of an AML xenograft in vivo. Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphoma and leukaemia (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2012, 86:348-357). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV (Bisgrove et al., Proc. Natl. Acad. Sci. USA, 2007, 104:13690-13695).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123).

BET proteins also regulate the expression of the ApoA1 gene which plays an important role in atherosclerosis and inflammatory processes (Chung et al., J. Med. Chem, 2011, 54:3827-3838). Apolipoprotein A1 (ApoA1) is a major component of high density lipoproteins (HDL), and increased expression of ApoA1 leads to elevated blood cholesterol values (Degoma and Rader, Nat. Rev. Cardiol., 2011, 8:266-277). Elevated HDL values are associated with a reduced risk of atherosclerosis (Chapman et al., Eur. Heart J., 2011, 32:1345-1361).

All these studies show that the BET proteins play an essential role in various pathologies, and also in male fertility. It would therefore be desirable to find potent and selective inhibitors which prevent the interaction between the BET proteins and acetylated proteins, in particular acetylated histone H4 peptides.

### PRIOR ART

The nomenclature employed in the assessment of the structural prior art is illustrated by the following figure:

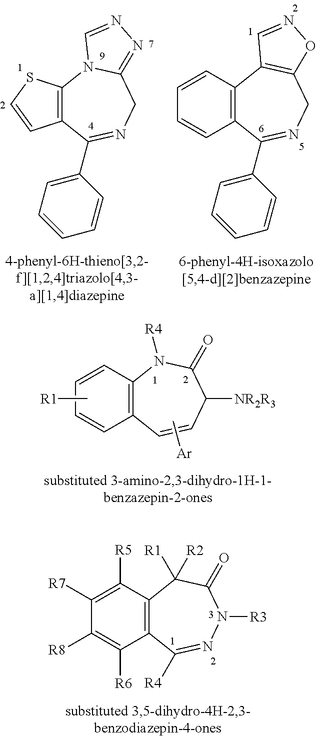

Based on the chemical structure, some types of BRD4 inhibitors have been described to date (Chun-Wa Chung et al., Progress in Medicinal Chemistry 2012, 51, 1-55).

The first published BRD4 inhibitors are phenylthienotriazolo-1,4-diazepines (4-phenyl-6H-thieno[3,2-f][1,2,4]triazolo[4,3-a][1,4]diazepines) as described in WO2009/084693 (Mitsubishi Tanabe Pharma Corporation) and as compound JQ1 in WO2011/143669 (Dana Farber Cancer Institute). Replacement of the thieno moiety by a benzo moiety also leads to active inhibitors (J. Med. Chem. 2011, 54, 3827-3838; E. Nicodeme et al., Nature 2010, 468, 1119). These and one further publication show that the pyrazole unit fused to the 1,4-benzodiazepine or thieno-1,4-diazepine ring system is actively involved in binding of the target protein BRD4 (P. Filippakopoulos et al., Nature 2010, 468, 1067). Further 4-phenyl-6H-thieno[3,2-f][1,2,4]triazolo[4,3-a][1,4]diazepines and related compounds having alternative rings as a fusion partner rather than the benzo unit are addressed generically or described directly in WO2012/075456 (Constellation Pharmaceuticals). WO2012/075383 (Constellation Pharmaceuticals) describes 6-substituted 4H-isoxazolo[5,4-d][2]benzazepines and 4H-isoxazolo[3,4-d][2]benzazepines, including compounds which have optionally substituted phenyl at position 6 as BRD4 inhibitors, and also analogues with alternative heterocyclic fusion partners rather than the benzo unit, for example thieno- or pyridoazepines. WO2013/184876 and WO2013/184878 (Constellation Pharmaceuticals) describe further benzoisoxazoloazepine derivatives as inhibitors of proteins comprising bromo domains.

Another structural class of BRD4 inhibitors described is that of 7-isoxazoloquinolines and related quinolone derivatives (WO2011/054843, Bioorganic & Medicinal Chemistry Letters 22 (2012) 2963-2967, GlaxoSmithKline). Pyridinones and pyridazinones (WO 2013/185284, WO 2013/188381; Abbott Laboratories) and also isoindolones (WO 2013/155695 and WO 2013/158952; Abbott Laboratories) have been described as inhibitors of binding of the bromo domains of the BET proteins to proteins comprising N-acetylated lysine residues.

WO94/26718/EP0703222A1 (Yoshitomi Pharmaceutical Industries) describes substituted 3-amino-2,3-dihydro-1H-1-benzazepin-2-ones or the corresponding 2-thiones and analogues in which the benzo unit has been replaced by alternative monocyclic systems, and in which the 2-ketone or the 2-thione together with the substituted nitrogen atom in the azepine ring may form a heterocycle, as CCK and gastrin antagonists for the treatment of CNS disorders, such as states of anxiety and depression, and of pancreatic disorders and of gastrointestinal ulcers. Ligands of the gastrin and the cholecystokinin receptor are described in WO2006/051312 (James Black Foundation). They also include substituted 3,5-dihydro-4H-2,3-benzodiazepin-4-ones which differ from the compounds according to the invention mainly by the obligatory oxo group in position 4 and by an obligatory carbonyl group-containing alkyl chain in position 5. Finally, substituted 3,5-dihydro-4H-2,3-benzodiazepin-4-ones are also described as AMPA antagonists in WO97/34878 (Cocensys Inc.). The generic claim is very wide with respect to the possible substitution patterns at the benzodiazepine skeleton; however, the working examples are limited to a very narrow range.

It would therefore be desirable to provide novel compounds having prophylactic and therapeutic properties.

Accordingly, it is an object of the present invention to provide compounds and pharmaceutical compositions comprising these compounds used for prophylactic and therapeutic applications for hyperproliferative disorders, in particular for tumour disorders, and as BET protein inhibitors for viral infections, for neurodegenerative disorders, for inflammatory disorders, for atherosclerotic disorders and for male fertility control.

The compounds according to the invention are novel phenyl-2,3-benzodiazepines (1-phenyl-4,5-dihydro-3H-2,3-benzodiazepines) and heteroaryl-2,3-benzodiazepines (1-heteroaryl-4,5-dihydro-3H-2,3-benzodiazepines) which are not fused at the benzodiazepine skeleton to a second heterocyclic moiety, specifically an isoxazole or triazole, and are still, surprisingly, BRD4 inhibitors.

Furthermore, the compounds according to the invention differ from known 2,3-benzodiazepines such as the numerous published AMPA antagonists (WO0198280, Annovis Inc.; WO 9728135, Schering AG; for a review, see Med. Res. Rev. 2007, 27(2), 239-278) or from analogous diazepines where the benzo moiety is replaced by a different monocyclic moiety by their substitution pattern at the phenyl group or at the benzo moiety or another monocyclic moiety: at least one substituent at the phenyl group or at the benzo moiety is cyclic ((hetero)aromatic, (hetero)cyclic) or is new at the position in question, for example trifluoromethoxy or alkylaminosulphonylphenyl at the benzo moiety.

The compounds according to the invention also differ from the known psychopharmacological 2,3-benzodiazepine derivatives which are inhibitors of the adenosine transporter and the MT2 receptor (WO2008/124075, Teva Pharm).

The structurally most similar compounds of the prior art have not been disclosed in the context of the prophylaxis and therapy of tumor disorders.

From the prior art described above, there was no reason to modify the structures of the prior art such that structures suitable for the prophylaxis and therapy of tumour disorders are obtained.

Surprisingly, the compounds according to the invention inhibit the interaction between BET proteins, in particular BRD4, and an acetylated histone 4 peptide and inhibit the growth of cancer cells. Accordingly, they provide novel structures for the therapy of human and animal disorders, in particular of cancers.

It has now been found that compounds of the general formula (I)

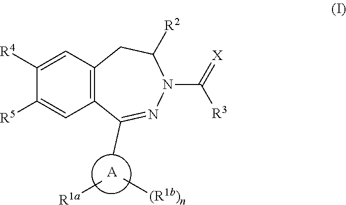

in which
X represents an oxygen or sulphur atom,
A represents a monocyclic heteroaryl ring having 5 or 6 ring atoms,
   or
   represents a phenyl ring,
R^{1a} represents a spirocycloalkyl, heterospirocycloalkyl, bicycloalkyl, heterobicycloalkyl radical, a bridged cycloalkyl radical or a bridged heterocycloalkyl radical, a naphthyl radical or a bicyclic heteroaryl radical, or a partially saturated bicyclic aryl- or heteroaryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical having 3 to 8 ring atoms,
n represents 0, 1 or 2,
R^{1b} represents halogen, hydroxy, cyano, nitro or represents a C₁-C₆-alkyl-, C₁-C₆-alkoxy-, C₁-C₆-alkoxy-C₁-C₆-alkyl-, halo-C₁-C₆-alkyl-, halo-C₁-C₆-alkoxy-, C₃-C₁₀-cycloalkyl radical or a monocyclic heterocyclyl radical having 3 to 8 ring atoms,
R² represents a C₁-C₃-alkyl or trifluoromethyl or a C₃- or C₄-cycloalkyl radical,
R³ represents cyclopropyl-, C₁-C₃-alkyl-, C₁-C₃-alkoxy-, amino-, cyclopropylamino- or C₁-C₃-alkylamino-,
R⁴ and R⁵ independently of one another represent hydrogen, hydroxy, cyano, nitro, amino, aminocarbonyl-, fluorine, chlorine or bromine,
   or
   represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms where the monocyclic heterocyclyl and heteroaryl radicals mentioned may for their part optionally be monosubstituted by C₁-C₃-alkyl,
   or
   represent C₃-C₁₀-cycloalkyl- which may optionally be mono- or polysubstituted by identical or different substituents from the group consisting of halogen, amino, hydroxy, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₃-C₆-alkoxy- and a monocyclic heterocyclyl radical having 3 to 8 ring atoms,
   or
   represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy-, C₃-C₁₀-cycloalkyl and a monocyclic heterocyclyl radical having 3 to 8 ring atoms,
   or
   represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represent phenyl- which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms where phenyl-, heteroaryl- and heterocyclyl- may optionally be mono- or disubstituted by halogen, C₁-C₃-alkoxy- or C₁-C₃-alkyl-,
R⁹ represents C₁-C₆-alkyl-,
   and their polymorphs, enantiomers, diastereomers, racemates, tautomers, solvates, physiologically acceptable salts and solvates of these salts are particularly suitable for a large number of prophylactic and therapeutic applications, in particular for hyperproliferative disorders, for tumour disorders and as BET proteine inhibitors, in particular as inhibitors of BRD4, for viral infections, for neurodegenerative disorders, for inflammatory disorders, for atherosclerotic disorders and for male fertility control.

Preference is given to those compounds of the general formula I in which
X represents an oxygen atom,
A represents a monocyclic heteroaryl ring having 6 ring atoms which may contain one or two nitrogen atoms,
   or
   represents a phenyl ring,
R^{1a} represents a heterospirocycloalkyl, heterobicycloalkyl radical or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
n is 0, 1 or 2,
R^{1b} represents halogen, hydroxy, cyano, nitro or represents a C₁-C₆-alkyl-, C₁-C₆-alkoxy-, C₁-C₆-alkoxy-C₁-C₆-alkyl-, halo-C₁-C₆-alkyl-, halo-C₁-C₆-alkoxy-, C₃-C₁₀-cycloalkyl radical or a monocyclic heterocyclyl radical having 3 to 8 ring atoms,
R² represents methyl,
R³ represents cyclopropyl-, C₁-C₃-alkyl-, C₁-C₃-alkoxy-, amino-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
   or
   represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- or amino-C₁-C₆-alkyl-, monocyclic heterocyclyl-having 3 to 8 ring atoms and monocyclic heteroaryl- having 5 or 6 ring atoms where the monocyclic heterocyclyl and heteroaryl radicals mentioned may for their part optionally be monosubstituted by C₁-C₃-alkyl,
   or
   represent C₃-C₁₀-cycloalkyl- which may optionally be mono- or polysubstituted by identical or different substituents from the group consisting of halogen, amino, hydroxy, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
   or
   represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represent phenyl- which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl, C₁-C₆-alkylaminosulphonyl-, C₁-C₆-alkylamino-C₁-C₆-alkyl, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl, monocyclic heterocyclyl- having 3 to 8 ring atoms or monocyclic heteroaryl- having 5 or 6 ring atoms,
R⁹ represents C₁-C₆-alkyl-,
   and their polymorphs, enantiomers, diastereomers, racemates, tautomers, solvates, physiologically acceptable salts and solvates of these salts.

More preference is given to those compounds of the general formula I in which
X represents an oxygen atom,
A represents a phenyl ring,
R^{1a} represents a heterospirocycloalkyl, heterobicycloalkyl radical or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₁-C₆-alkyl-, C₁-C₆-alkylamino-C₁-C₆-alkyl-, fluoro-C₁-C₆-alkyl-, fluoro-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
n represents 0 or 1,
R^{1b} represents halogen, hydroxy, cyano, nitro or represents a C₁-C₃-alkyl-, C₁-C₃-alkoxy-, C₁-C₃-alkoxy-C₁-C₃-alkyl-, fluoro-C₁-C₃-alkyl-, fluoro-C₁-C₃-alkoxy-, C₃-C₇-cycloalkyl radical or a monocyclic heterocyclyl radical having 5 or 6 ring atoms,
R² represents methyl,
R³ represents cyclopropyl-, C₁-C₃-alkyl-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
   or
   represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
   or
   represent C₃-C₇-cycloalkyl- which may optionally be mono- or polysubstituted by identical or different substituents from the group consisting of halogen, amino, hydroxy, carboxy, C₁-C₃-alkyl-, C₁-C₃-alkoxy-, C₁-C₃-alkylamino-, amino-C₁-C₃-alkyl-, fluoro-C₁-C₃-alkyl-, fluoro-C₁-C₃-alkoxy- and a monocyclic heterocyclyl radical having 5 or 6 ring atoms,
   or
   represent monocyclic heteroaryl having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, hydroxy-C₁-C₃-alkyl-, C₁-C₃-alkylamino-, amino-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
   or
   represent monocyclic heterocyclyl having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, C₁-C₃-alkylamino-, amino-C₁-C₃-alkyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
   or
   represents phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylamino-, amino-C₁-C₃-alkyl-, C₁-C₃-alkylaminocarbonyl, C₁-C₃-alkylaminosulphonyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, fluoro-C₁-C₃-alkyl-, hydroxy-C₁-C₃-alkyl-, C₃-C₈-cycloalkyl-, phenyl, monocyclic heterocyclyl- having 3 to 8 ring atoms or monocyclic heteroaryl- having 5 or 6 ring atoms,
R⁹ represents C₁-C₆-alkyl-,
   and their polymorphs, enantiomers, diastereomers, racemates, tautomers, solvates, physiologically acceptable salts and solvates of these salts.

Even more preference is given to those compounds of the general formula I in which
X represents an oxygen atom,
A represents a phenyl ring,
R^{1a} represents a heterospirocycloalkyl-, heterobicycloalkyl- or a bridged heterocycloalkyl radical, a naphthyl radical or a bicyclic heteroaryl radical, or a partially saturated bicyclic aryl radical where the radicals mentioned may optionally be mono- or polysubstituted by identical or different substituents from the group consisting of halogen, cyano, nitro, hydroxy, amino, oxo, carboxy, C₁-C₄-alkyl-, C₁-C₄-alkoxy-, hydroxy-C₁-C₄-alkyl-, C₁-C₄-alkylamino-, C₁-C₄-alkylcarbonylamino-, amino-C₁-C₄-alkyl-, fluoro-C₁-C₄-alkyl-, fluoro-C₁-C₄-alkoxy-, C₃-C₈-cycloalkyl-, phenyl-, halophenyl-, phenyl-C₁-C₄-alkyl-, phenoxy-, pyridinyl-, —C(═O)—NR⁶R⁷, —C(═O)—R⁸, —S(═O)₂—NR⁶R⁷, —S(═O)₂—R⁹, —NH—S(═O)₂—R⁹ and a monocyclic heterocyclyl radical having 3 to 8 ring atoms,
n represents 0 or 1,
R^{1b} represents halogen, hydroxy, cyano, nitro or represents a C₁-C₃-alkyl-, C₁-C₃-alkoxy-, C₁-C₃-alkoxy-C₁-C₃-alkyl-, fluoro-C₁-C₃-alkyl-, fluoro-C₁-C₃-alkoxy-, C₃-C₇-cycloalkyl radical or a monocyclic heterocyclyl radical having 5 or 6 ring atoms,
R² represents methyl,
R³ represents cyclopropyl-, C₁-C₃-alkyl-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
   or
   represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
   or
   represent monocyclic heteroaryl having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, hydroxy-C₁-C₃-alkyl-, C₁-C₃-alkylamino-, amino-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
   or
   represent monocyclic heterocyclyl having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, C₁-C₃-alkylamino-, amino-C₁-C₃-alkyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
   or
   represent phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylamino-, amino-C₁-C₃-alkyl-, C₁-C₃-alkylaminocarbonyl, C₁-C₃-alkylaminosulphonyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₄-alkyl-, C₁-C₄-alkoxy-, fluoro-C₁-C₃-alkyl-, hydroxy-C₁-C₃-alkyl-, C₃-C₈-cycloalkyl-, phenyl, monocyclic heterocyclyl- having 3 to 8 ring atoms or monocyclic heteroaryl- having 5 or 6 ring atoms,
R⁹ represents C₁-C₄-alkyl-,
   and their polymorphs, enantiomers, diastereomers, racemates, tautomers, solvates, physiologically acceptable salts and solvates of these salts.

Particular preference is given to those compounds of the general formula I in which
X represents an oxygen atom,
A represents a phenyl ring,
R^{1a} represents a heterospirocycloalkyl-, heterobicycloalkyl- or a bridged heterocycloalkyl radical, a naphthyl radical or a bicyclic heteroaryl radical, or a partially saturated bicyclic aryl radical where the radicals mentioned may optionally be mono- or polysubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl-, halophenyl-, phenyl-C₁-C₂-alkyl, pyridinyl-, phenoxy- and —C(═O)—R⁸,
n represents 0 or 1,
R^{1b} represents halogen, hydroxy, cyano, or represents a C₁-C₃-alkyl-, C₁-C₃-alkoxy-, fluoro-C₁-C₃-alkyl- or fluoro-C₁-C₃-alkoxy radical,
R² represents methyl,
R³ represents C₁-C₃-alkyl- or C₁-C₃-alkylamino-,
R⁴ and R⁵ independently of one another represent hydrogen, hydroxy, cyano, aminocarbonyl-, fluorine, chlorine, bromine,
   or
   represent C₁-C₄-alkyl-, C₁-C₄-alkoxy-, C₁-C₆-alkylamino-, C₁-C₄-alkylcarbonylamino-, C₁-C₄-alkylaminocarbonyl- or C₁-C₄-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
   or
   represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, fluoro-C₁-C₃-alkyl- and fluoro-C₁-C₃-alkoxy-,
   or
   represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, fluoro-C₁-C₃-alkyl- and fluoro-C₁-C₃-alkoxy-,
   or
   represents phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylaminocarbonyl, C₁-C₃-alkylaminosulphonyl-, fluoro-C₁-C₃-alkyl- and fluoro-C₁-C₃-alkoxy-,
   R⁸ represents hydroxy, C₁-C₄-alkyl-, C₁-C₄-alkoxy-, fluoro-C₁-C₃-alkyl-, hydroxy-C₁-C₃-alkyl-, C₃-C₈-cycloalkyl-, phenyl, monocyclic heterocyclyl- having 4 to 7 ring atoms or monocyclic heteroaryl- having 5 or 6 ring atoms,
      and their polymorphs, enantiomers, diastereomers, racemates, tautomers, solvates, physiologically acceptable salts and solvates of these salts.

Very particular preference is given to those compounds of the general formula I in which
X represents an oxygen atom,
A represents a phenyl ring,
R^{1a} represents the radicals bicyclo[2.2.1]heptyl-, spiro[3.3]heptyl-, bicyclo[3.2.1]octyl-, spiro[3.4]octyl-, bicyclo[4.2.1]nonyl-, spiro[3.5]nonyl-, spiro[4.5]decyl- which contain one, two or three identical or different heteroatoms selected from the group consisting of oxygen, nitrogen and sulphur and which may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl, phenyl-C₁-C₂-alkyl, phenoxy- and —C(═O)—R⁸,
   or
   represents the radicals perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl- which may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl, phenyl-C₁-C₂-alkyl, phenoxy- and —C(═O)—R⁸,
n represents 0 or 1,
R^{5a} represents fluorine, chlorine or cyano,
R² represents methyl,
R³ represents methyl or C₁-C₃-alkylamino-,
R⁴ and R⁵ independently of one another represent hydrogen, hydroxy, cyano, aminocarbonyl-, fluorine, chlorine, bromine,
   or
   represent C₁-C₄-alkyl-, C₁-C₄-alkoxy- which may optionally be mono- or polysubstituted by identical or different substituents from the group consisting of fluorine, amino, hydroxy, carboxy, C₁-C₃-alkoxy,
   or
   represent monocyclic heteroaryl- having 5 or 6 ring atoms which may optionally be mono- or polysubstituted by identical or different substituents from the group consisting of halogen, cyano, nitro, C₁-C₃-alkyl-, C₁-C₃-alkoxy-, fluoro-C₁-C₃-alkyl- and fluoro-C₁-C₃-alkoxy-,
   or
   represent monocyclic heterocyclyl- having 4 to 7 ring atoms which may optionally be mono- or polysubstituted by identical or different substituents from the group consisting of halogen, cyano, oxo, C₁-C₃-alkyl-, C₁-C₃-alkoxy-, fluoro-C₁-C₃-alkyl- and fluoro-C₁-C₃-alkoxy-,
   or
   represent phenyl which may optionally be mono- or polysubstituted by identical or different substituents from the group consisting of halogen, cyano, C₁-C₃-alkyl-, C₁-C₃-alkoxy-, fluoro-C₁-C₃-alkyl- and fluoro-C₁-C₃-alkoxy-,
R⁸ represents C₁-C₄-alkyl or C₁-C₄-alkoxy-,
   and their polymorphs, enantiomers, diastereomers, racemates, tautomers, solvates, physiologically acceptable salts and solvates of these salts.

Very particular preference is also given to those compounds of the general formula I in which
X represents an oxygen atom,
A represents a phenyl ring,
R^{1a} represents the radicals bicyclo[2.2.1]heptyl-, spiro[3.3]heptyl-, bicyclo[3.2.1]octyl-, spiro[3.4]octyl-, bicyclo[4.2.1]nonyl-, spiro[3.5]nonyl-, spiro[4.5]decyl- which contain one, two or three identical or different heteroatoms selected from the group consisting of oxygen, nitrogen and sulphur and which may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl, monocyclic heterocyclyl- having 3 to 8 ring members, phenyl, halophenyl-, phenyl-C₁-C₂-alkyl-, phenoxy- and —C(═O)—R⁸,
   or
   represents the radicals perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl- which may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl, monocyclic heterocyclyl- having 3 to 8 ring members, phenyl, halophenyl-, phenyl-C₁-C₂-alkyl-, phenoxy- and —C(═O)—R⁸,
n represents 0 or 1,
R^{1b} represents fluorine, chlorine or cyano,
R² represents methyl,
R³ represents methyl or C₁-C₃-alkylamino-,
R⁴ represents hydrogen or C₁-C₃-alkoxy-,
R⁵ represents hydrogen, C₁-C₃-alkoxy or fluoro-C₁-C₃-alkoxy-,
   or
   represents a monocyclic heteroaryl radical having 5 or 6 ring atoms which may be mono- or disubstituted by C₁-C₃-alkyl, C₁-C₃-alkoxy- or halogen,
R⁸ represents C₁-C₄-alkyl or C₁-C₄-alkoxy-,
   and their polymorphs, enantiomers, diastereomers, racemates, tautomers, solvates, physiologically acceptable salts and solvates of these salts.

Most preference is given to those compounds of the general formula I in which
X represents an oxygen atom,
A represents a phenyl ring,
R^{1a} represents the radicals 2-azabicyclo[2.2.1]heptyl-, 2,5-diazabicyclo[2.2.1]heptyl-, 2-oxa-5-azabicyclo[2.2.1]heptyl-, 2-azaspiro[3.3]heptyl-, 1-thia-6-azaspiro[3.3]heptyl-, 2-thia-6-azaspiro[3.3]heptyl-, 2-oxa-6-azaspiro[3.3]heptyl-, 2,6-diazaspiro[3.3]heptyl-, 8-oxa-3-azabicyclo[3.2.1]octyl-, 8-azabicyclo[3.2.1]octyl-, 2-oxa-6-azaspiro[3.4]octyl-, 3,9-diazabicyclo[4.2.1]nonyl-, 2-oxa-6-azaspiro[3.5]nonyl-, 2-oxa-7-azaspiro[3.5]nonyl-, 8-azaspiro[4.5]decyl-, 2,8-diazaspiro[4.5]decyl-, 3-oxa-1,8-diazaspiro[4.5]decyl-, perhydrofuro[3,2-c]pyridinyl-, perhydropyrrolo[1,2-a]pyrazinyl-, perhydropyrrolo[3,4-c]pyrrolyl-, quinolinyl-, isochinolinyl-, indolyl-, 2,3-dihydro-1,4-benzodioxinyl-, 1,3-benzodioxolyl-, 2,3-dihydro-1-benzofuranyl-, where the radicals mentioned may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl-, monocyclic heterocyclyl- having 3 to 8 ring members, phenyl, halophenyl-, phenyl-C₁-C₂-alkyl-, phenoxy- and —C(═O)—R⁸,
n represents 0 or 1,
R^{1b} represents fluorine,
R² represents methyl,
R³ represents methyl or C₁-C₃-alkylamino-,
R⁴ represents hydrogen or C₁-C₃-alkoxy-,
R⁵ represents hydrogen, C₁-C₃-alkoxy- or fluoro-C₁-C₃-alkoxy-,
   or
   represents a monocyclic heteroaryl radical having 5 ring atoms which contains at least one nitrogen atom through which it is attached to the remainder of the molecule, and which may be mono- or disubstituted by C₁-C₃-alkyl or halogen,
R⁸ represents C₁-C₄-alkyl or C₁-C₄-alkoxy-,
   and their polymorphs, enantiomers, diastereomers, racemates, tautomers, solvates, physiologically acceptable salts and solvates of these salts.

Particularly interesting compounds of the general formula (I) are those in which
X represents an oxygen atom,
A represents a phenyl ring,
R^{1a} represents the radicals

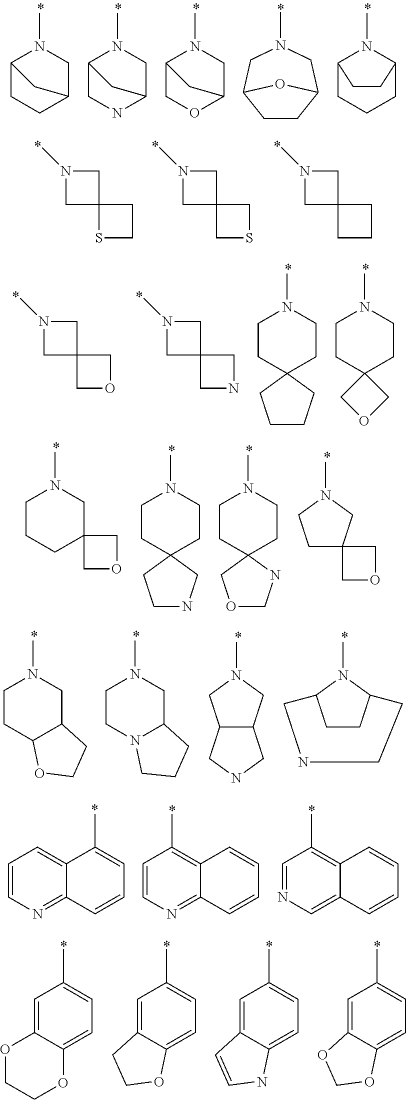

where “*” denotes the point of attachment to the remainder of the molecule,
and the radicals may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl-, phenyl-C₁-C₂-alkyl-, phenoxy- and —C(═O)—R⁸,
n represents 0 or 1,
R^{1b} represents fluorine,
R² represents methyl,
R³ represents C₁-C₃-alkylamino-,
R⁴ represents hydrogen or methoxy-,
R⁵ represents methoxy-, trifluoromethoxy- or 3,5-dimethylpyrazol-1-yl,
R⁸ represents C₁-C₄-alkyl or C₁-C₄-alkoxy,
   and their polymorphs, enantiomers, diastereomers, racemates, tautomers, solvates, physiologically acceptable salts and solvates of these salts.

Likewise of particular interest are those compounds of the general formula I in which
X represents an oxygen atom,
A represents a phenyl ring,
R^{1a} represents the radicals

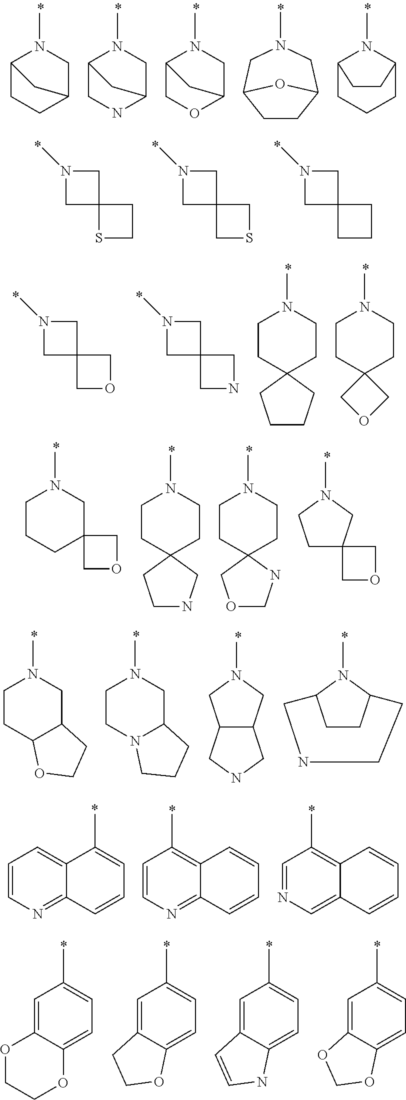

where “*” denotes the point of attachment to the remainder of the molecule and the radicals may optionally be mono- or disubstituted by identical or different radicals from the group consisting of oxo, fluorine, chlorine, bromine, cyano, hydroxy, methyl, ethyl, methoxy-, ethoxy-, benzyl-, phenyl-, phenoxy- and —C(═O)—R⁸,
n represents 0 or 1,
R^{1b} represents fluorine,
R² represents methyl,
R³ represents C₁-C₃-alkylamino-,
R⁴ represents hydrogen or methoxy-,
R⁵ represents methoxy-, trifluoromethoxy- or 3,5-dimethylpyrazol-1-yl,
R⁸ represents methyl or tert-butoxy-,
   and their polymorphs, enantiomers, diastereomers, racemates, tautomers, solvates, physiologically acceptable salts and solvates of these salts.

Of very particular interest are those compounds of the general formula (I)

in which
X represents an oxygen atom,
A represents a phenyl ring,
R^{1a} represents the radicals

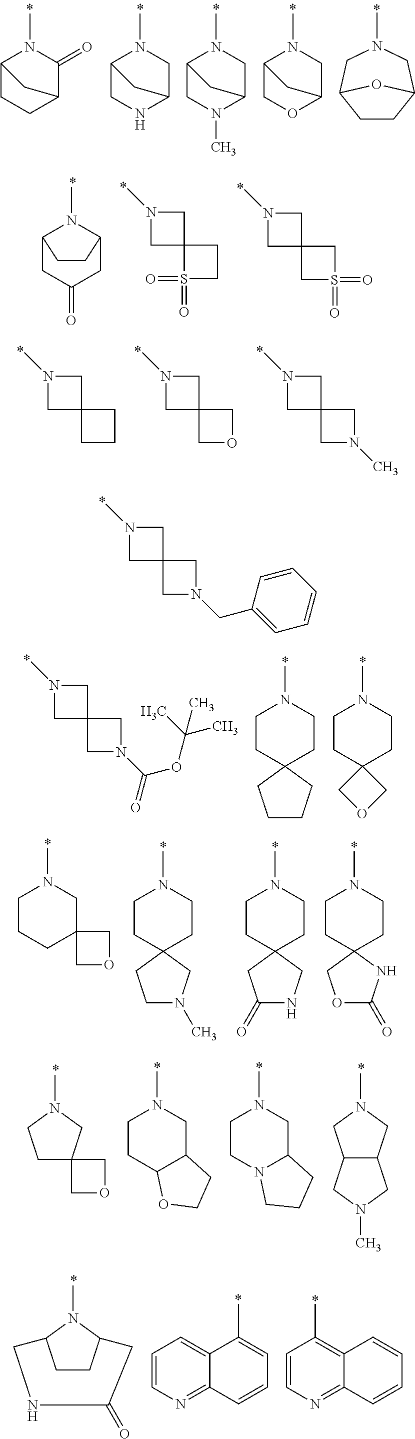

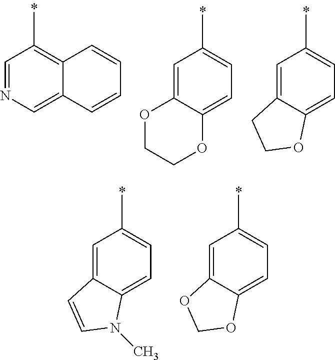

where “*” denotes the point of attachment to the remainder of the molecule,
n represents 0 or 1,
R^{1b} represents fluorine,
R² represents methyl-,
R³ represents methylamino-,
R⁴ represents hydrogen or methoxy-,
R⁵ represents methoxy-, trifluoromethoxy- or 3,5-dimethylpyrazol-1-yl,
   and their polymorphs, enantiomers, diastereomers, racemates, tautomers, solvates, physiologically acceptable salts and solvates of these salts.

Preference is furthermore given to those compounds of the general formula I in which
X represents an oxygen or sulphur atom,
A represents a monocyclic heteroaryl ring having 5 or 6 ring atoms or represents a phenyl ring,
R^{1a} represents a spirocycloalkyl, heterospirocycloalkyl, bicycloalkyl, heterobicycloalkyl radical, a bridged cycloalkyl radical or a bridged heterocycloalkyl radical, a naphthyl radical or a bicyclic heteroaryl radical, or a partially saturated bicyclic aryl or heteroaryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₆-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
n represents 0, 1 or 2 and
R^{1b} represents halogen, hydroxy, cyano, nitro and/or represents a C₁-C₆-alkyl-, C₁-C₆-alkoxy-, C₁-C₆-alkoxy-C₁-C₆-alkyl-, halo-C₁-C₆-alkyl-, halo-C₁-C₆-alkoxy-, C₃-C₁₀-cycloalkyl radical and/or a monocyclic heterocyclyl radical having 3 to 8 ring atoms,
R² represents a C₁-C₃-alkyl or trifluoromethyl or a C₃- or C₄-cycloalkyl radical, and
R³ represents cyclopropyl-, C₁-C₃-alkyl-, C₁-C₃-alkoxy-, amino-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
   or
   represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms where the monocyclic heterocyclyl and heteroaryl radicals mentioned may for their part optionally be monosubstituted by C₁-C₃-alkyl,
   or
   represent C₃-C₁₀-cycloalkyl- which may optionally be mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₆-C₆-alkyl-, C₁-C₆-alkylamino-C₁-C₆-alkyl, halo-C₁-C₆-alkyl-, halo-C₆-C₆-alkoxy- and a monocyclic heterocyclyl radical having 3 to 8 ring atoms,
   or
   represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy-, C₃-C₁₀-cycloalkyl and a monocyclic heterocyclyl radical having 3 to 8 ring atoms,
   or
   represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represent phenyl- which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms where phenyl-, heteroaryl- and heterocyclyl- may optionally be mono- or disubstituted by halogen, C₁-C₃-alkoxy- or C₁-C₃-alkyl-,
R⁹ represents C₁-C₆-alkyl-,
   and their polymorphs, enantiomers, diastereomers, racemates, tautomers, solvates, physiologically acceptable salts and solvates of these salts.

Very particular preference is furthermore given to those compounds of the general formula I in which
X represents an oxygen atom,
A represents a phenyl ring,
R^{1a} represents a group selected from the group consisting of bicyclo[2.2.1]heptyl-, spiro[3.3]heptyl-, bicyclo[3.2.1]octyl-, spiro[3.4]octyl-, spiro[4.5]decyl-, where the groups mentioned independently of one another contain at least one, optionally two, heteroatoms selected from the group consisting of oxygen, nitrogen and sulphur which may be identical or different,
   or represents a group selected from the group consisting of octahydrofuro[3,2-c]pyridinyl, octahydropyrrolo[1,2-a]pyrazinyl, quinolinyl, isoquinolinyl, 2,3-dihydro-1,4-benzodioxinyl, 2,3-dihydro-1-benzofuranyl,
   where the groups mentioned in each case may optionally independently of one another be mono- or disubstituted by identical or different radicals from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl having 3 to 8 ring members, phenyl, halophenyl, phenyl-C₁-C₂-alkyl-, phenoxy- and —C(═O)—R⁸,
n represents 0 or 1,
R^{1b} represents fluorine, chlorine or cyano,
R² represents methyl and
R³ represents methyl or C₁-C₃-alkylamino-,
R⁴ and R⁵ independently of one another represent hydrogen, hydroxy, cyano, aminocarbonyl-, fluorine, chlorine, bromine,
   or
   represent C₁-C₄-alkyl-, C₁-C₄-alkoxy- which may optionally be mono- or polysubstituted by identical or different substituents from the group consisting of fluorine, amino, hydroxy, carboxy, C₁-C₃-alkoxy,
   or
   represent a monocyclic heteroaryl radical having 5 or 6 ring atoms which may optionally be mono- or polysubstituted by identical or different substituents from the group consisting of halogen, cyano, nitro, C₁-C₃-alkyl-, C₁-C₃-alkoxy-, fluoro-C₁-C₃-alkyl- and fluoro-C₁-C₃-alkoxy-,
   or
   represent a monocyclic heterocyclyl radical having 4 to 7 ring atoms which may optionally be mono- or polysubstituted by identical or different substituents from the group consisting of halogen, cyano, oxo, C₁-C₃-alkyl-, C₁-C₃-alkoxy-, fluoro-C₁-C₃-alkyl- and fluoro-C₁-C₃-alkoxy-,
   or
   represent a phenyl radical which may optionally be mono- or polysubstituted by identical or different substituents from the group consisting of halogen, cyano, C₁-C₃-alkyl-, C₁-C₃-alkoxy-, fluoro-C₁-C₃-alkyl- and fluoro-C₁-C₃-alkoxy-,
R⁸ represents C₁-C₄-alkyl or C₁-C₄-alkoxy-,
   and their polymorphs, enantiomers, diastereomers, racemates, tautomers, solvates, physiologically acceptable salts and solvates of these salts.

Very particular preference is furthermore also given to those compounds of the general formula I in which
X represents an oxygen atom,
A represents a phenyl ring,
R^{1a} represents a group selected from the group consisting of bicyclo[2.2.1]heptyl-, spiro[3.3]heptyl-, bicyclo[3.2.1]octyl-, spiro[3.4]octyl-, spiro[4.5]decyl-, where the groups mentioned independently of one another each contain at least one, optionally two, heteroatoms selected from the group consisting of oxygen, nitrogen and sulphur which may be identical or different,
   or represents a group selected from the group consisting of octahydrofuro[3,2-c]pyridinyl, octahydropyrrolo[1,2-a]pyrazinyl, quinolinyl, isoquinolinyl, 2,3-dihydro-1,4-benzodioxinyl, 2,3-dihydro-1-benzofuranyl,
   where the groups mentioned in each case may optionally independently of one another be mono- or disubstituted by identical or different radicals from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl-, monocyclic heterocyclyl having 3 to 8 ring members, phenyl, halophenyl-, phenyl-C₁-C₂-alkyl-, phenoxy- and —C(═O)—R⁸,
n represents 0 or 1,
R^{1b} represents fluorine, chlorine or cyano,
R² represents methyl,
R³ represents methyl or C₁-C₃-alkylamino-,
R⁴ represents hydrogen or C₁-C₃-alkoxy-,
R⁵ represents hydrogen, C₁-C₃-alkoxy or fluoro-C₁-C₃-alkoxy-, or represents a heteroaryl radical having 5 or 6 ring atoms which may be mono- or disubstituted by C₁-C₃-alkyl, C₁-C₃-alkoxy- and/or halogen, and
R⁸ represents C₁-C₄-alkyl or C₁-C₄-alkoxy-,
   and their polymorphs, enantiomers, diastereomers, racemates, tautomers, solvates, physiologically acceptable salts and solvates of these salts.

Extraordinary preference is furthermore given to those compounds of the general formula I in which
X represents an oxygen atom,
A represents a phenyl ring,
R^{1a} represents a group selected from the group consisting of 2-azabicyclo[2.2.1]heptyl-, 1-thia-6-azaspiro[3.3]heptyl-, 8-oxa-3-azabicyclo[3.2.1]octyl-, 2-oxa-6-azaspiro[3.3]heptyl-, 8-azabicyclo[3.2.1]octyl-, octahydrofuro[3,2-c]-pyridinyl-, 2,5-diazabicyclo[2.2.1]heptyl-, 2,6-diazaspiro[3.3]heptyl-, 2-oxa-6-azaspiro[3.4]octyl-, 8-azaspiro[4.5]decyl-, 2,8-diazaspiro[4.5]decyl-, octahydropyrrolo[1,2-a]pyrazinyl-, quinolinyl-, isoquinolinyl-, 2,3-dihydro-1,4-benzodioxinyl-, 2,3-dihydro-1-benzofuranyl-, where the groups mentioned may optionally independently of one another be mono- or disubstituted by identical or different radicals from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl-, monocyclic heterocyclyl having 3 to 8 ring members, phenyl, halophenyl-, phenyl-C₁-C₂-alkyl-, phenoxy- and —C(═O)—R⁸,
n represents 0 or 1,
R^{1b} represents fluorine,
R² represents methyl,
R³ represents methyl or C₁-C₃-alkylamino-,
R⁴ represents hydrogen or C₁-C₃-alkoxy-,
R⁵ represents hydrogen, C₁-C₃-alkoxy- or fluoro-C₁-C₃-alkoxy-, or represents a heteroaryl radical having 5 ring atoms which contains at least one nitrogen atom through which it is attached to the remainder of the molecule, and which may be mono- or disubstituted by C₁-C₃-alkyl and/or halogen, and
R⁸ represents C₁-C₄-alkyl or C₁-C₄-alkoxy-,
   and their polymorphs, enantiomers, diastereomers, racemates, tautomers, solvates, physiologically acceptable salts and solvates of these salts.

Particularly interesting compounds of the general formula (I) are furthermore those in which
X represents an oxygen atom,
A represents a phenyl ring,
R¹ represents a group selected from

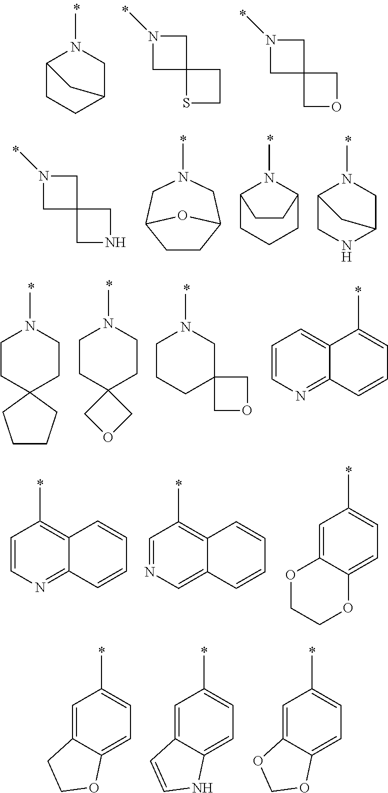

where “*” denotes the point of attachment to the remainder of the molecule,
where the groups may optionally independently of one another be mono- or disubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl having 3 to 8 ring members, phenyl, halophenyl-, phenyl-C₁-C₂-alkyl-, phenoxy- and —C(═O)—R⁸,
n represents 0 or 1,
R^{1b} represents fluorine,
R² represents methyl,
R³ represents C₁-C₃-alkylamino-,
R⁴ represents hydrogen or methoxy-,
R⁵ represents methoxy-, trifluoromethoxy- or 3,5-dimethylpyrazol-1-yl, and
R⁸ represents C₁-C₄-alkyl or C₁-C₄-alkoxy,
   and their polymorphs, enantiomers, diastereomers, racemates, tautomers, solvates, physiologically acceptable salts and solvates of these salts.

Likewise of particular interest are furthermore those compounds of the general formula I in which
X represents an oxygen atom,
A represents a phenyl ring,
R¹ represents a group selected from

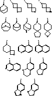

where “*” denotes the point of attachment to the remainder of the molecule,
and the groups may optionally independently of one another be mono- or disubstituted by identical or different radicals from the group consisting of oxo, fluorine, chlorine, bromine, cyano, hydroxy, methyl, ethyl, methoxy-, ethoxy-, benzyl-, phenyl-, phenoxy- and —C(═O)—R⁸,
n represents 0 or 1,
R^{1b} represents fluorine,
R² represents methyl,
R³ represents C₁-C₃-alkylamino-,
R⁴ represents hydrogen or methoxy-,
R⁵ represents methoxy-, trifluoromethoxy- or 3,5-dimethylpyrazol-1-yl, and
R⁸ represents methyl or tert-butoxy-,
   and their polymorphs, enantiomers, diastereomers, racemates, tautomers, solvates, physiologically acceptable salts and solvates of these salts.

Of very particular interest are furthermore those compounds of the general formula (I) in which
X represents an oxygen atom,
A represents a phenyl ring,
R¹ represents a group selected from

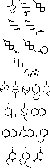

where “*” denotes the point of attachment to the remainder of the molecule,
n represents 0 or 1,
R^{1b} represents fluorine,
R² represents methyl-,
R³ represents methylamino-,
R⁴ represents hydrogen or methoxy-, and
R⁵ represents methoxy-, trifluoromethoxy- or 3,5-dimethylpyrazol-1-yl,
   and their polymorphs, enantiomers, diastereomers, racemates, tautomers, solvates, physiologically acceptable salts and solvates of these salts.

Of special interest are furthermore those compounds of the general formula (I) in which
X represents an oxygen atom,
A represents a phenyl ring,
R¹ represents a group selected from

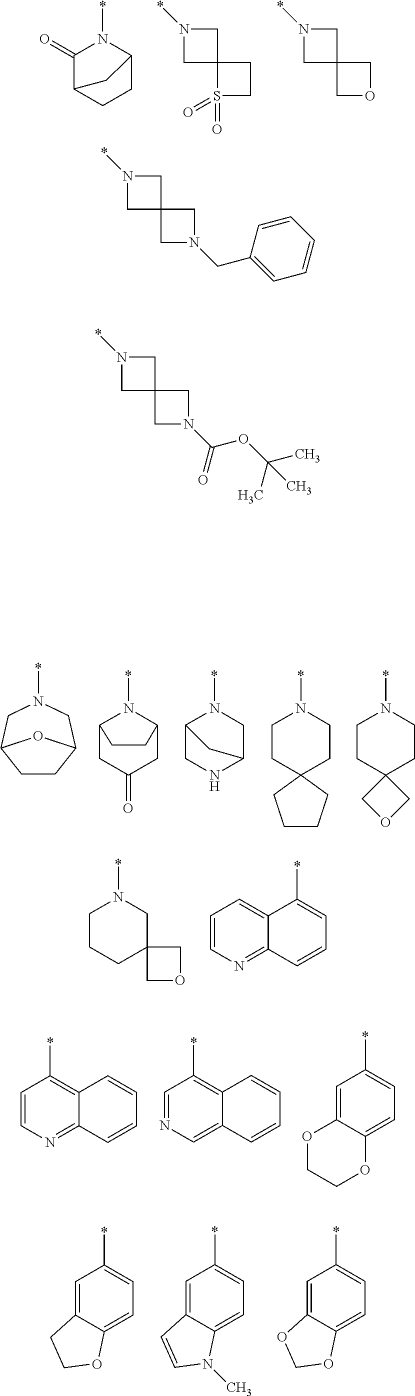

where “*” denotes the point of attachment to the remainder of the molecule,
n represents 0 or 1,
R^{1b} represents fluorine,
R² represents methyl-,
R³ represents methylamino-,
R⁴ represents hydrogen or methoxy-, and
R⁵ represents methoxy-, trifluoromethoxy- or 3,5-dimethylpyrazol-1-yl,
   and their polymorphs, enantiomers, diastereomers, racemates, tautomers, solvates, physiologically acceptable salts and solvates of these salts.

Extraordinary preference is given to the following compounds:
[1S-(1R*,4S*)]-7,8-dimethoxy-N,4-dimethyl-1-[4-(3-oxo-2-azabicyclo[2.2.1]hept-2-yl)phenyl]-4,5-dihydro-3H-2,3-benzodiazepine-3-carboxamide;
{1S-[1R*,2(S*),4S*]}-7,8-dimethoxy-N,4-dimethyl-1-[4-(-3-oxo-2-azabicyclo[2.2.1]hept-2-yl)phenyl]-4,5-dihydro-3H-2,3-benzodiazepine-3-carboxamide;
(4S)-1-[4-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(4R)-1-[4-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(±)-1-[4-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(8-oxa-3-azabicyclo[3.2.1]oct-3-yl)phenyl]-4,5-dihydro-3H-2,3-benzodiazepine-3-carboxamide;
(4R)-7,8-dimethoxy-N,4-dimethyl-1-[4-(8-oxa-3-azabicyclo[3.2.1]oct-3-yl)phenyl]-4,5-dihydro-3H-2,3-benzodiazepine-3-carboxamide;
(4S)-7,8-dimethoxy-N,4-dimethyl-1-[4-(8-oxa-3-azabicyclo[3.2.1]oct-3-yl)phenyl]-4,5-dihydro-3H-2,3-benzodiazepine-3-carboxamide;
(±)-7,8-dimethoxy-N,4-dimethyl-1-[4-(2-oxa-6-azaspiro[3.3]hept-6-yl)phenyl]-4,5-dihydro-3H-2,3-benzodiazepine-3-carboxamide;
(4S)-7,8-dimethoxy-N,4-dimethyl-1-[4-(2-oxa-6-azaspiro[3.3]hept-6-yl)phenyl]-4,5-dihydro-3H-2,3-benzodiazepine-3-carboxamide;
(4R)-7,8-dimethoxy-N,4-dimethyl-1-[4-(2-oxa-6-azaspiro[3.3]hept-6-yl)phenyl]-4,5-dihydro-3H-2,3-benzodiazepine-3-carboxamide;
(±)-7,8-dimethoxy-N,4-dimethyl-1-[4-(3-oxo-8-azabicyclo[3.2.1]oct-8-yl)phenyl]-4,5-dihydro-3H-2,3-benzodiazepine-3-carboxamide;
(4R)-7,8-dimethoxy-N,4-dimethyl-1-[4-(3-oxo-8-azabicyclo[3.2.1]oct-8-yl)phenyl]-4,5-dihydro-3H-2,3-benzodiazepine-3-carboxamide;
(4S)-7,8-dimethoxy-N,4-dimethyl-1-[4-(3-oxo-8-azabicyclo[3.2.1]oct-8-yl)phenyl]-4,5-dihydro-3H-2,3-benzodiazepine-3-carboxamide;
tert-butyl 6-{4-[(±)-7,8-dimethoxy-4-methyl-3-(methylcarbamoyl)-4,5-dihydro-3H-2,3-benzodiazepine-1-yl]phenyl}-2,6-diazaspiro[3.3]heptane-2-carboxylate;
tert-butyl 6-{4-[(4S)-7,8-dimethoxy-4-methy-3-(methylcarbamoyl)-4,5-dihydro-3H-2,3-benzodiazepine-1-yl]phenyl}-2,6-diazaspiro[3.3]heptane-2-carboxylate;
tert-butyl 6-{4-[(4R)-7,8-dimethoxy-4-methyl-3-(methylcarbamoyl)-4,5-dihydro-3H-2,3-benzodiazepine-1-yl]phenyl}-2,6-diazaspiro[3.3]heptane-2-carboxylate;
(±)-1-[4-(8-azaspiro[4.5]dec-8-yl)phenyl]-7,8-dimethoxy-N,4-dimethyl-4,5-dihydro-3H-2,3-benzodiazepine-3-carboxamide;
(4S)-1-[4-(8-azaspiro[4.5]dec-8-yl)phenyl]-7,8-dimethoxy-N,4-dimethyl-4,5-dihydro-3H-2,3-benzodiazepine-3-carboxamide;
(4R)-1-[4-(8-azaspiro[4.5]dec-8-yl)phenyl]-7,8-dimethoxy-N,4-dimethyl-4,5-dihydro-3H-2,3-benzodiazepine-3-carboxamide;
(±)-1-[4-(6-benzyl-2,6-diazaspiro[3.3]hept-2-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(2-oxa-6-azaspiro[3.5]non-6-yl)phenyl]-4,5-dihydro-3H-2,3-benzodiazepine-3-carboxamide;
(±)-7,8-dimethoxy-N,4-dimethyl-1-[4-(2-oxa-7-azaspiro[3.5]non-7-yl)phenyl]-4,5-dihydro-3H-2,3-benzodiazepine-3-carboxamide;
(±)-7,8-dimethoxy-N,4-dimethyl-1-[3-(8-oxa-3-azabicyclo[3.2.1]oct-3-yl)phenyl]-4,5-dihydro-3H-2,3-benzodiazepine-3-carboxamide;
(±)-1-[3-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(±)-1-[3-(1,1-dioxido-1-thia-6-azaspiro[3.3]hept-6-yl)-4-fluorophenyl]-7,8-dimethoxy-N,4-dimethyl-4,5-dihydro-3H-2,3-benzodiazepine-3-carboxamide;
(±)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide;
(4R)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide;
(4S)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide;
(±)-1-[4-(1,1-dioxido-1-thia-6-azaspiro[3.3]hept-6-yl)phenyl]-N,4-dimethyl-8-(trifluoromethoxy)-4,5-dihydro-3H-2,3-benzodiazepine-3-carboxamide;
(4R)-1-[4-(1,1-dioxido-1-thia-6-azaspiro[3.3]hept-6-yl)phenyl]-N,4-dimethyl-8-(trifluoromethoxy)-4,5-dihydro-3H-2,3-benzodiazepine-3-carboxamide;
(4S)-1-[4-(1,1-dioxido-1-thia-6-azaspiro[3.3]hept-6-yl)phenyl]-N,4-dimethyl-8-(trifluoromethoxy)-4,5-dihydro-3H-2,3-benzodiazepine-3-carboxamide;
[1S-(1R*,4R*)]-1-[4-(2,5-diazabicyclo[2.2.1]hept-2-yl)phenyl]-7,8-dimethoxy-4,5-dihydro-N,4-dimethyl-3H-2,3-benzodiazepine-3-carboxamide;
[1S-(1R*,4R*)]-1-[3-(2,5-diazabicyclo[2.2.1]hept-2-yl)-4-fluorophenyl]-7,8-dimethoxy-4,5-dihydro-N,4-dimethyl-3H-2,3-benzodiazepine-3-carboxamide;
(±)-1-[4-(2,3-dihydro-1,4-benzodioxin-6-yl)phenyl]-7,8-dimethoxy-N,4-dimethyl-4,5-dihydro-3H-2,3-benzodiazepine-3-carboxamide;
(±)-1-[4-(2,3-dihydro-1-benzofuran-5-yl)phenyl]-7,8-dimethoxy-N,4-dimethyl-4,5-dihydro-3H-2,3-benzodiazepine-3-carboxamide;
(±)-1-[4-(quinolin-5-yl)phenyl]-7,8-dimethoxy-N,4-dimethyl-4,5-dihydro-3H-2,3-benzodiazepine-3-carboxamide;
(±)-1-[4-(quinolin-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(1-methyl-1H-indol-5-yl)phenyl]-4,5-dihydro-3H-2,3-benzodiazepine-3-carboxamide;
(±)-1-[4-(isoquinolin-4-yl)phenyl]-7,8-dimethoxy-N,4-dimethyl-4,5-dihydro-3H-2,3-benzodiazepine-3-carboxamide;
(±)-1-[4-(1,3-benzodioxol-5-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(3-oxo-2,8-diazaspiro[4.5]dec-8-yl)phenyl]-4,5-dihydro-3H-2,3-benzodiazepine-3-carboxamide;
(±)-7,8-dimethoxy-N,4-dimethyl-1-[4-(2-oxa-6-azaspiro[3.4]oct-6-yl)phenyl]-4,5-dihydro-3H-2,3-benzodiazepine-3-carboxamide;
(±)-1-[4-(hexahydropyrrolo[1,2-a]pyrazin-2(1H)-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(2-methyl-2,8-diazaspiro[4.5]dec-8-yl)phenyl]-4,5-dihydro-3H-2,3-benzodiazepine-3-carboxamide;
(±)-7,8-dimethoxy-N,4-dimethyl-1-[4-(2-oxo-3-oxa-1,8-diazaspiro[4.5]dec-8-yl)phenyl]-4,5-dihydro-3H-2,3-benzodiazepine-3-carboxamide;
(±)-7,8-dimethoxy-N,4-dimethyl-1-{4-[(3aR,6aS)-5-methylhexahydropyrrolo[3,4-c]pyrrol-2(1H)-yl]phenyl}-4,5-dihydro-3H-2,3-benzodiazepine-3-carboxamide;
(±)-1-[4-(2,2-dioxido-2-thia-6-azaspiro[3.3]hept-6-yl)phenyl]-7,8-dimethoxy-N,4-dimethyl-4,5-dihydro-3H-2,3-benzodiazepine-3-carboxamide;
(4S)-7,8-dimethoxy-N,4-dimethyl-1-{4-[(1S,4S)-2-oxa-5-azabicyclo[2.2.1]hept-5-yl]phenyl}-4,5-dihydro-3H-2,3-benzodiazepine-3-carboxamide;
(±)-1-[4-(hexahydrofuro[3,2-c]pyridin-5(4H-yl)phenyl]-7,8-dimethoxy-N,4-dimethyl-4,5-dihydro-3H-2,3-benzodiazepine-3-carboxamide;
(±)-1-[4-(2-azaspiro[3.3]hept-2-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(6-methyl-2,6-diazaspiro[3.3]hept-2-yl)phenyl]-4,5-dihydro-3H-2,3-benzodiazepine-3-carboxamide;
(±)-7,8-dimethoxy-N,4-dimethyl-1-[4-(4-oxo-3,9-diazabicyclo[4.2.1]non-9-yl)phenyl]-4,5-dihydro-3H-2,3-benzodiazepine-3-carboxamide;
[1S-(1R*,4R*)]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide;
[1S-[1R*,2(S*),4R*]]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide;
[1S-[1R*,2(R*),4R*]]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide;
[1S-(1R*,4R*)]-1-[4-fluoro-3-(5-methyl-2,5-diazabicyclo[2.2.1]hept-2-yl)phenyl]-7,8-dimethoxy-4,5-dihydro-N,4-dimethyl-3H-2,3-benzodiazepine-3-carboxamide;
[1S-(1R*,2(R*),4R*)]-1-[4-fluoro-3-(5-methyl-2,5-diazabicyclo[2.2.1]hept-2-yl)phenyl]-7,8-dimethoxy-4,5-dihydro-N,4-dimethyl-3H-2,3-benzodiazepine-3-carboxamide
and
[1S-(1R*,2(S*),4R*)]-1-[4-fluoro-3-(5-methyl-2,5-diazabicyclo[2.2.1]hept-2-yl)phenyl]-7,8-dimethoxy-4,5-dihydro-N,4-dimethyl-3H-2,3-benzodiazepine-3-carboxamide.

In the general formula (I), X may represent an oxygen or sulphur atom.

In the general formula (I), X preferably represents an oxygen atom.

In the general formula (I), A may represent a monocyclic heteroaryl ring having 5 or 6 ring atoms or a phenyl ring.

In the general formula (I), A preferably represents a monocyclic 6-membered heteroaryl ring which may contain one or two nitrogen atoms, or represents a phenyl ring.

In the general formula (I), A particularly preferably represents a phenyl ring.

In the general formula (I), R^{1a} preferably represents a heterospirocycloalkyl, heterobicycloalkyl radical or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms.

In the general formula (I), R^{1a} even more preferably represents a heterospirocycloalkyl, heterobicycloalkyl or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of halogen, cyano, nitro, hydroxy, amino, oxo, carboxy, C₁-C₄-alkyl-, C₁-C₄-alkoxy-, hydroxy-C₁-C₄-alkyl-, C₁-C₄-alkylamino-, C₁-C₄-alkylcarbonylamino-, amino-C₁-C₄-alkyl-, fluoro-C₁-C₄-alkyl-, fluoro-C₁-C₄-alkoxy-, C₃-C₈-cycloalkyl-, phenyl-, halophenyl-, phenyl-C₁-C₄-alkyl-, phenoxy-, pyridinyl-, —C(═O)—NR⁶R⁷, —C(═O)—R⁸, —S(═O)₂—NR⁶R⁷, —S(═O)₂—R⁹, —NH—S(═O)₂—R⁹ and a monocyclic heterocyclyl radical having 3 to 8 ring atoms.

In the general formula (I), R^{1a} even more preferably represents a heterospirocycloalkyl, heterobicycloalkyl or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of oxo, fluoro, chloro, bromo, cyano, hydroxy, methyl, ethyl, methoxy-, ethoxy-, benzyl-, phenyl-, phenoxy- and —C(═O)—R⁸.

In the general formula (I), R^{1a} even more preferably represents the radicals bicyclo[2.2.1]heptyl-, spiro[3.3]heptyl-, bicyclo[3.2.1]octyl-, spiro[3.4]octyl-, bicyclo[4.2.1]nonyl-, spiro[3.5]nonyl-, spiro[4.5]decyl- which contain one, two or three identical or different heteroatoms selected from the group consisting of oxygen, nitrogen and sulphur and which may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl, phenyl-C₁-C₂-alkyl, phenoxy- and —C(═O)—R⁸, or

represents the radicals perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl- which may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl, phenyl-C₁-C₂-alkyl, phenoxy- and —C(═O)—R⁸.

In the general formula (I), R^{1a} also very particularly preferably represents the radicals bicyclo[2.2.1]heptyl-, spiro[3.3]heptyl-, bicyclo[3.2.1]octyl-, spiro[3.4]octyl-, bicyclo[4.2.1]nonyl-, spiro[3.5]nonyl-, spiro[4.5]decyl- which contain one, two or three identical or different heteroatoms selected from the group consisting of oxygen, nitrogen and sulphur and which may optionally be mono- or disubstituted by identical or different radicals from the group consisting of oxo, fluorine, chlorine, bromine, cyano, hydroxy, methyl, ethyl, methoxy-, ethoxy-, benzyl-, phenyl-, phenoxy- and —C(═O)—R⁸, or

represents the radicals perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl- which may optionally be mono- or disubstituted by identical or different substituents from the group consisting of oxo, fluorine, chlorine, bromine, cyano, hydroxy, methyl, ethyl, methoxy-, ethoxy-, benzyl-, phenyl-, phenoxy- and —C(═O)—R⁸.

In the general formula (I), R^{1a} further very particularly preferably represents a group selected from the group consisting of bicyclo[2.2.1]heptyl-, spiro[3.3]heptyl-, bicyclo[3.2.1]octyl-, spiro[3.4]octyl-, spiro[4.5]decyl-, where the groups mentioned independently of one another contain at least one, optionally also two, heteroatoms selected from the group consisting of oxygen, nitrogen and sulphur which may be identical or different,

or represents a group selected from the group consisting of octahydrofuro[3,2-c]pyridinyl-, octahydropyrrolo[1,2-a]pyrazinyl-, quinolinyl-, isoquinolinyl-, 2,3-dihydro-1,4-benzodioxinyl-, 2,3-dihydro-1-benzofuranyl-,
where the groups mentioned in each case may optionally independently of one another be mono- or disubstituted by identical or different radicals from the group consisting of oxo, halogen, cyano, hydroxy, C₁-C₄-alkyl-, fluoro-C₁-C₄-alkyl-, C₁-C₄-alkoxy-, fluoro-C₁-C₄-alkoxy, C₃-C₈-cycloalkyl-, monocyclic heterocyclyl- having 3 to 8 ring members, phenyl-, halophenyl-, phenyl-C₁-C₂-alkyl-, phenoxy- and —C(═O)—R⁸,

In the general formula (I), R^{1a} with extraordinary preference represents the radicals 2-azabicyclo[2.2.1]heptyl-, 2,5-diazabicyclo[2.2.1]heptyl-, 2-oxa-5-azabicyclo[2.2.1]heptyl-, 2-azaspiro[3.3]heptyl-, 1-thia-6-azaspiro[3.3]heptyl-, 2-thia-6-azaspiro[3.3]heptyl-, 2-oxa-6-azaspiro[3.3]heptyl-, 2,6-diazaspiro[3.3]heptyl-, 8-oxa-3-azabicyclo[3.2.1]octyl-, 8-azabicyclo[3.2.1]octyl-, 2-oxa-6-azaspiro[3.4]octyl-, 3,9-diazabicyclo[4.2.1]nonyl-, 2-oxa-6-azaspiro[3.5]nonyl-, 2-oxa-7-azaspiro[3.5]nonyl-, 8-azaspiro[4.5]decyl-, 2,8-diazaspiro[4.5]decyl-, 3-oxa-1,8-diazaspiro[4.5]decyl-, perhydrofuro[3,2-c]pyridinyl-, perhydropyrrolo[1,2-a]pyrazinyl-, perhydropyrrolo[3,4-c]pyrrolyl-, quinolinyl-, isochinolinyl-, indolyl-, 2,3-dihydro-1,4-benzodioxinyl-, 1,3-benzodioxolyl-, 2,3-dihydro-1-benzofuranyl- where the radicals mentioned may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl-, monocyclic heterocyclyl- having 3 to 8 ring members, phenyl, halophenyl-, phenyl-C₁-C₂-alkyl-, phenoxy- and —C(═O)—R⁸.

In the general formula (I), R^{1a} furthermore with extraordinary preference represents the radicals 2-azabicyclo[2.2.1]heptyl-, 2,5-diazabicyclo[2.2.1]heptyl-, 2-oxa-5-azabicyclo[2.2.1]heptyl-, 2-azaspiro[3.3]heptyl-, 1-thia-6-azaspiro[3.3]heptyl-, 2-thia-6-azaspiro[3.3]heptyl-, 2-oxa-6-azaspiro[3.3]heptyl-, 2,6-diazaspiro[3.3]heptyl-, 8-oxa-3-azabicyclo[3.2.1]octyl-, 8-azabicyclo[3.2.1]octyl-, 2-oxa-6-azaspiro[3.4]octyl-, 3,9-diazabicyclo[4.2.1]nonyl-, 2-oxa-6-azaspiro[3.5]nonyl-, 2-oxa-7-azaspiro[3.5]nonyl-, 8-azaspiro[4.5]decyl-, 2,8-diazaspiro[4.5]decyl-, 3-oxa-1,8-diazaspiro[4.5]decyl-, perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl-, where the radicals mentioned may optionally be mono- or disubstituted by identical or different substituents from the group consisting of oxo, fluoro, chloro, bromo, cyano, hydroxy, methyl, ethyl, methoxy-, ethoxy-, benzyl-, phenyl-, phenoxy- and —C(═O)—R⁸.

In the general formula (I), of particular interest are those compounds in which

R^{1a} represents the radicals

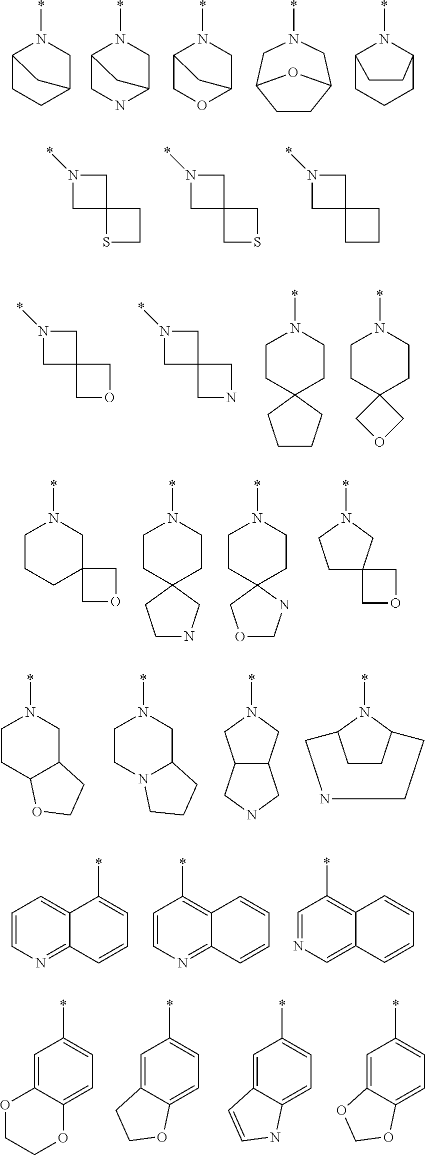

where “*” denotes the point of attachment to the remainder of the molecule,
and the radicals may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl having 3 to 8 ring members, phenyl, halophenyl-, phenyl-C₁-C₂-alkyl-, phenoxy- and —C(═O)—R⁸.

In the general formula (I), also of particular interest are those compounds in which
R^{1a} represents the radicals

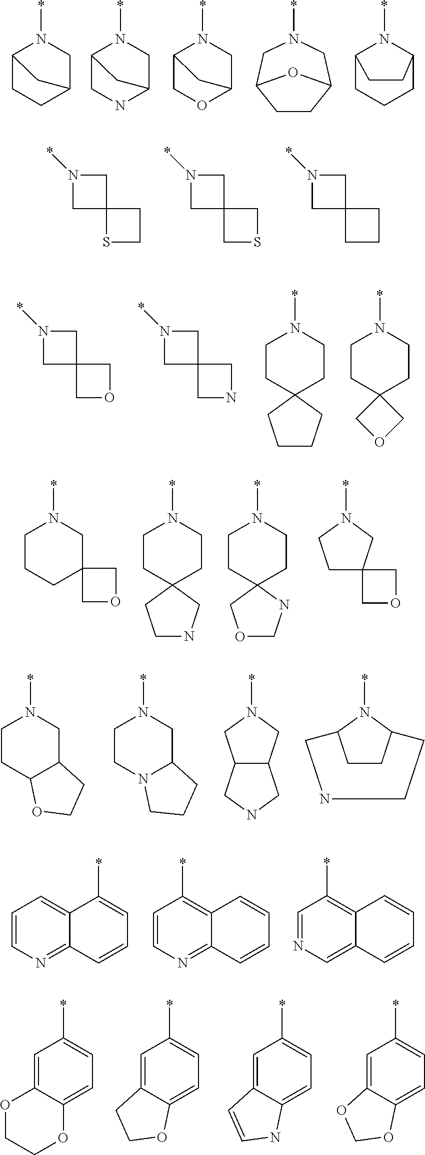

where “*” denotes the point of attachment to the remainder of the molecule,
and the radicals may optionally be mono- or disubstituted by identical or different radicals from the group consisting of oxo, fluorine, chlorine, bromine, cyano, hydroxy, methyl, ethyl, methoxy-, ethoxy-, benzyl-, phenyl-, phenoxy- and —C(═O)—R⁸.

In the general formula (I), furthermore of particular interest are those compounds in which
R^{1a} represents a group selected from

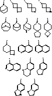

where “*” denotes the point of attachment to the remainder of the molecule,
and the groups may optionally be mono- or disubstituted independently of one another by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl having 3 to 8 ring members, phenyl, halophenyl-, phenyl-C₁-C₂-alkyl-, phenoxy- and —C(═O)—R⁸.

In the general formula (I), of very particular interest are those compounds in which R^{1a} represents the radicals

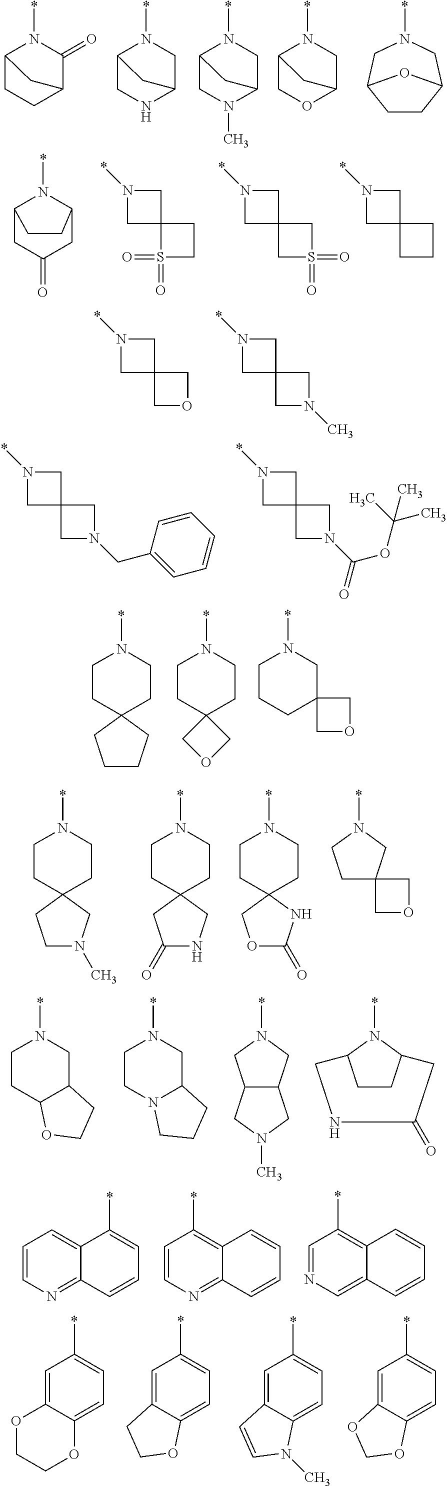

In the general formula (I), R^{1b} preferably represents halogen, hydroxy, cyano, nitro or represents a C₁-C₆-alkyl-, C₁-C₆-alkoxy-, C₁-C₆-alkoxy-C₁-C₆-alkyl-, halo-C₁-C₆-alkyl-, halo-C₁-C₆-alkoxy-, C₃-C₁₀-cycloalkyl radical or a monocyclic heterocyclyl radical having 3 to 8 ring atoms.

In the general formula (I), R^{1b} particularly preferably represents halogen, hydroxy, cyano, or represents a C₁-C₃-alkyl-, C₁-C₃-alkoxy-, fluoro-C₁-C₃-alkyl- or fluoro-C₁-C₃-alkoxy radical.

In the general formula (I), R^{1b} very particularly preferably represents fluorine, chlorine or cyano.

In the general formula (I), R^{1b} with extraordinary preference represents fluorine.

In the general formula (I), n may represent 0, 1 or 2.

In the general formula (I), n particularly preferably represents 0 or 1.

In the general formula (I), n particularly preferably represents 1.

In the general formula (I), n especially preferably represents 0.

In the general formula (I), R² may represent a C₁-C₃-alkyl- or trifluoromethyl- or a C₃- or C₄-cycloalkyl radical.

In the general formula (I), R² preferably represents a methyl radical.

In the general formula (I), R³ may represent a cyclopropyl-, C₁-C₃-alkyl-, C₁-C₃-alkoxy-, amino-, cyclopropylamino- or a C₁-C₃-alkylamino radical.

In the general formula (I), R³ particularly preferably represents a C₁-C₃-alkyl or a C₁-C₃-alkylamino radical.

In the general formula (I), R³ very particularly preferably represents a methyl or a C₁-C₃-alkylamino radical.

In the general formula (I), R³ very particularly preferably represents a methyl radical.

In the general formula (I), R³ very particularly preferably represents a C₁-C₃-alkylamino radical.

In the general formula (I), of very particular interest are those compounds in which R³ represents a methylamino radical.

In the general formula (I), R⁴ and R⁵ independently of one another may represent hydrogen, hydroxy, cyano, nitro, amino, aminocarbonyl-, fluorine, chlorine, bromine,

or
C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms where the monocyclic heterocyclyl and heteroaryl radicals mentioned may for their part optionally be monosubstituted by C₁-C₃-alkyl,
or
represent C₃-C₁₀-cycloalkyl- which may optionally be mono- or polysubstituted by identical or different substituents from the group consisting of halogen, amino, hydroxy, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
or
represents monocyclic heteroaryl- having 5 or 6 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy-, C₃-C₁₀-cycloalkyl and a monocyclic heterocyclyl radical having 3 to 8 ring atoms,
or
represents monocyclic heterocyclyl- having 3 to 8 ring atoms which may optionally be mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
or
represents phenyl- which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,

In the general formula (I), R⁴ and R⁵ independently of one another more preferably represent hydrogen, hydroxy, cyano, nitro, amino, aminocarbonyl-, fluorine, chlorine, bromine,

or
represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
or
represent C₃-C₇-cycloalkyl- which may optionally be mono- or polysubstituted by identical or different substituents from the group consisting of halogen, amino, hydroxy, carboxy, C₁-C₃-alkyl-, C₁-C₃-alkoxy-, C₁-C₃-alkylamino-, amino-C₁-C₃-alkyl-, fluoro-C₁-C₃-alkyl-, fluoro-C₁-C₃-alkoxy- and a monocyclic heterocyclyl radical having 5 or 6 ring atoms,
or
represent monocyclic heteroaryl having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, hydroxy-C₁-C₃-alkyl-, C₁-C₃-alkylamino-, amino-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
or
represent monocyclic heterocyclyl having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, C₁-C₃-alkylamino-, amino-C₁-C₃-alkyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
or
represent phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylamino-, amino-C₁-C₃-alkyl-, C₁-C₃-alkylaminocarbonyl, C₁-C₃-alkylaminosulphonyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms.

In the general formula (I), R⁴ and R⁵ independently of one another very particularly preferably represent hydrogen, hydroxy, cyano, aminocarbonyl-, fluorine, chlorine, bromine,

or
represent C₁-C₄-alkyl-, C₁-C₄-alkoxy- which may optionally be mono- or polysubstituted by identical or different substituents from the group consisting of fluorine, amino, hydroxy, carboxy, C₁-C₃-alkoxy,
or
represent monocyclic heteroaryl- having 5 or 6 ring atoms which may optionally be mono- or polysubstituted by identical or different substituents from the group consisting of halogen, cyano, nitro, C₁-C₃-alkyl-, C₁-C₃-alkoxy-, fluoro-C₁-C₃-alkyl- and fluoro-C₁-C₃-alkoxy-,
or
represent monocyclic heterocyclyl- having 4 to 7 ring atoms which may optionally be mono- or polysubstituted by identical or different substituents from the group consisting of halogen, cyano, oxo, C₁-C₃-alkyl-, C₁-C₃-alkoxy-, fluoro-C₁-C₃-alkyl- and fluoro-C₁-C₃-alkoxy-,
or
represent phenyl which may optionally be mono- or polysubstituted by identical or different substituents from the group consisting of halogen, cyano, C₁-C₃-alkyl-, C₁-C₃-alkoxy-, fluoro-C₁-C₃-alkyl- and fluoro-C₁-C₃-alkoxy-.

In the general formula (I), R⁴ likewise very particularly preferably represents hydrogen or C₁-C₃-alkoxy-.

In the general formula (I), of very particular interest are those compounds in which R⁴ represents hydrogen or methoxy-.

In the general formula (I), R⁵ likewise very particularly preferably represents hydrogen, C₁-C₃-alkoxy or fluoro-C₁-C₃-alkoxy-, or represents a heteroaryl radical having 5 or 6 ring atoms which may be mono- or disubstituted by C₁-C₃-alkyl, C₁-C₃-alkoxy- or halogen.

In the general formula (I), R⁵ with extraordinary preference represents hydrogen, C₁-C₃-alkoxy- or fluoro-C₁-C₃-alkoxy-, or represents a heteroaryl radical having 5 ring atoms which contains at least one nitrogen atom through which it is attached to the remainder of the molecule, and which may be mono- or disubstituted by C₁-C₃-alkyl or halogen.

In the general formula (I), of very particular interest are those compounds in which R⁵ represents methoxy-, trifluoromethoxy- or 3,5-dimethylpyrazol-1-yl.

In the general formula (I), R⁶ and R⁷ preferably represent hydrogen, C₁-C₃-alkyl-, cyclopropyl- or di-C₁-C₃-alkylamino-C₁-C₃-alkyl-.

In the general formula (I), R⁸ preferably represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl, monocyclic heterocyclyl- having 3 to 8 ring atoms or monocyclic heteroaryl- having 5 or 6 ring atoms.

In the general formula (I), R⁸ particularly preferably represents hydroxy, C₁-C₄-alkyl-, C₁-C₄-alkoxy-, fluoro-C₁-C₃-alkyl-, hydroxy-C₁-C₃-alkyl-, C₃-C₈-cycloalkyl-, phenyl, monocyclic heterocyclyl- having 4 to 7 ring atoms or monocyclic heteroaryl- having 5 or 6 ring atoms.

In the general formula (I), R⁸ very particularly preferably represents C₁-C₄-alkyl or C₁-C₄-alkoxy-.

In the general formula (I), of very particular interest are those compounds in which R⁸ represents methyl or tert-butoxy-.

In the general formula (I), of special interest are those compounds in which R⁸ represents tert-butoxy-.

In the general formula (I), R⁹ preferably represents C₁-C₆-alkyl-.

In the general formula (I), R⁹ even more preferably represents C₁-C₄-alkyl-.

In the general formula (I), the stereocentre represented by the carbon atom, attached to R², of the benzodiazepine skeleton is present either in racemic form or predominantly or completely in the (S) configuration.

In the general formula (I), the stereocentre represented by the carbon atom, attached to R², of the benzodiazepine skeleton is preferably present in racemic form.

In the general formula (I), the stereocentre represented by the carbon atom, attached to R², of the benzodiazepine skeleton is particularly preferably present predominantly or completely in the (S) configuration.

In the general formula (I), the stereocentre represented by the carbon atom, attached to R², of the benzodiazepine skeleton is particularly preferably present predominantly in the (S) configuration.

In the general formula (I), the stereocentre represented by the carbon atom, attached to R², of the benzodiazepine skeleton is particularly preferably present completely in the (S) configuration.

The specific radical definitions given in the particular combinations or preferred combinations of radicals are, irrespective of the particular combinations of radicals specified, also replaced as desired by radical definitions of other combination.

Very particular preference is given to combinations of two or more of the abovementioned preferred ranges.

The invention is based on the following definitions:

In the present invention, the term “ring” may have the same meaning as the term “radical” which in this case also refers to a cyclic radical. Thus, for example, a monocyclic heteroaryl ring is to be understood as meaning a monocyclic heteroaryl radical.

### Alkyl

Alkyl represents a straight-chain or branched saturated monovalent hydrocarbon radical having generally 1 to 6 (C₁-C₆-alkyl), preferably 1 to 4 (C₁-C₄-alkyl) and particularly preferably 1 to 3 (C₁-C₃-alkyl) carbon atoms.

Examples which may be mentioned as being preferred are:

methyl-, ethyl-, propyl-, butyl-, pentyl-, hexyl-, isopropyl-, isobutyl-, sec-butyl, tert-butyl-, isopentyl-, 2-methylbutyl-, 1-methylbutyl-, 1-ethylpropyl-, 1,2-dimethylpropyl, neopentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, 1,2-dimethylbutyl-.

Particular preference is given to a methyl, ethyl, propyl, isopropyl or tert-butyl radical.

### Cycloalkyl

Cycloalkyl represents a monocyclic saturated monovalent hydrocarbon radical having generally 3 to 10 (C₃-C₁₀-cycloalkyl), preferably 3 to 8

(C₃-C₈-cycloalkyl) and particularly preferably 3 to 7 (C₃-C₇-cycloalkyl) carbon atoms.

Examples of monocyclic cycloalkyl radicals which may be mentioned as being preferred are:

cyclopropyl, cyclobutyl, cyclopentyl, cyclohexyl and cycloheptyl.

Particular preference is given to a cyclopropyl, cyclopentyl or cyclohexyl radical.

### Phenylalkyl

Phenyl-C₁-C₆-alkyl- is understood to mean a group composed of an optionally substituted phenyl radical and a C₁-C₆-alkyl group, and bonded to the rest of the molecule via the C₁-C₆-alkyl group.

Here, the alkyl radical has the meanings given above under alkyl.

Examples which may be mentioned include benzyl, phenethyl, phenylpropyl, phenylpentyl, with benzyl being preferred.

### Alkoxy

Alkoxy represents a straight-chain or branched saturated alkylether radical of the formula —O-alkyl having generally 1 to 6 (C₁-C₆-alkoxy), preferably 1 to 4 (C₁-C₄-alkoxy) and particularly preferably 1 to 3 (C₁-C₃-alkoxy) carbon atoms.

Examples which may be mentioned as being preferred are:

methoxy, ethoxy, n-propoxy, isopropoxy, tert-butoxy, n-pentyloxy and n-hexyloxy.

### Alkoxyalkyl

Alkoxyalkyl represents an alkyl radical substituted by alkoxy, for example C₁-C₆-alkoxy-C₁-C₆-alkyl- or C₁-C₃-alkoxy-C₁-C₃-alkyl-.

Here, C₁-C₆-alkoxy-C₁-C₆-alkyl- means that the alkoxyalkyl group is attached via the alkyl moiety to the remainder of the molecule.

### Oxo

Oxo, an oxo group or an oxo substituent, is understood to mean a double-bonded oxygen atom ═O.

Oxo may be attached to atoms of suitable valency, for example to a saturated carbon atom or to sulphur.

Preference is given to the bond to carbon with formation of a carbonyl group and to the bond of two double-bonded oxygen atoms to sulphur with formation of a sulphonyl group —(S═O)₂—.

### Alkylamino

Alkylamino represents an amino radical having one or two alkyl substituents (selected independently of one another) having generally 1 to 6 (C₁-C₆-alkylamino) and preferably 1 to 3 (C₁-C₃-alkylamino) carbon atoms.

(C₁-C₃)-Alkylamino represents, for example, a monoalkylamino radical having 1 to 3 carbon atoms or a dialkylamino radical having 1 to 3 carbon atoms each per alkyl substituent.

The following may be mentioned by way of example:

methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

### Alkylaminocarbonyl

Alkylaminocarbonyl represents the group alkylamino-C(═O)— having one or two alkyl substituents (selected independently of one another) having generally 1 to 6 (C₁-C₆-alkylaminocarbonyl) and preferably 1 to 3 (C₁-C₃-alkylaminocarbonyl) carbon atoms.

### Alkylcarbonylamino

Alkylcarbonylamino represents the group alkyl-C(═O)—NH— having generally 1 to 6 (C₁-C₆-alkylcarbonylamino), preferably 1 to 4 and particularly preferably 1 to 3 carbon atoms in the alkyl moiety.

### Alkylaminosulphonyl

Alkylaminosulphonyl represents the group alkylamino-S(═O)₂— having one or two alkyl substituents (selected independently of one another) having generally 1 to 6 (C₁-C₆-alkylaminosulphonyl) and preferably 1 to 3 carbon atoms.

Examples which may be mentioned as being preferred are:

methylaminosulphonyl, ethylaminosulphonyl, dimethylaminosulphonyl.

### Heteroatoms

Heteroatoms are understood to mean oxygen, nitrogen or sulphur atoms.

### Aryl

Aryl represents a monovalent mono- or bicyclic aromatic ring system which consists of carbon atoms. Examples are naphthyl- and phenyl-; preference is given to phenyl- or a phenyl radical.

### Halophenyl:

Halophenyl- refers to a phenyl radical which is mono- or polysubstituted by identical or different substituents from the group consisting of fluorine, chlorine and bromine.

### Heteroaryl

Heteroaryl represents a monovalent mono- or bicyclic aromatic ring system having one, two, three or four heteroatoms which may be identical or different. The heteroatoms may be nitrogen atoms, oxygen atoms or sulphur atoms. The binding valency can be at any aromatic carbon atom or at a nitrogen atom.

A monocyclic heteroaryl radical in accordance with the present invention has 5 or 6 ring atoms. Preference is given to heteroaryl radicals having one or two heteroatoms. Here, particular preference is given to one or two nitrogen atoms.

Heteroaryl radicals having 5 ring atoms include, for example, the rings:

thienyl, thiazolyl, furyl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the rings:

pyridinyl, pyridazinyl, pyrimidinyl, pyrazinyl and triazinyl.

A bicyclic heteroaryl radical in accordance with the present invention has 9 or 10 ring atoms.

Heteroaryl radicals having 9 ring atoms include, for example, the rings:

phthalidyl, thiophthalidyl, indolyl, isoindolyl, indazolyl, benzothiazolyl, benzofuryl, benzothienyl, benzimidazolyl, benzoxazolyl, azocinyl, indolizinyl, purinyl, indolinyl.

Heteroaryl radicals having 10 ring atoms include, for example, the rings:

isochinolinyl, quinolinyl, quinolizinyl, quinazolinyl, quinoxalinyl, cinnolinyl, phthalazinyl, 1,7- and 1,8-naphthyridinyl, pteridinyl, chromanyl.

### Partially Saturated Bicyclic Aryl and Partially Saturated Bicyclic Heteroaryl

A partially saturated bicyclic aryl radical or heteroaryl radical represents a bicyclic group consisting of a phenyl radical or a monocyclic 5- or 6-membered heteroaryl radical which is condensed via two directly adjacent ring atoms in each case to an aliphatic cyclic radical having 4 to 7 ring atoms which may optionally contain one or two heteroatoms which may be identical or different. The heteroatoms may be nitrogen atoms, oxygen atoms or sulphur atoms.

Partially saturated bicyclic aryl radicals include, for example, the groups:

tetrahydronaphthyl, 2,3-dihydro-1,4-benzodioxinyl-, 2,3-dihydro-1-benzofuranyl- and 1,3-benzodioxolyl-.

Partially saturated bicyclic heteroaryl radicals include, for example, the groups:

5,6,7,8-tetrahydroquinolinyl- and 5,6,7,8-tetrahydroisoquinolinyl-.

### Monocyclic Heterocyclyl

Monocyclic heterocyclyl- means a non-aromatic monocyclic ring system having one, two or three heteroatoms which may be identical or different. The heteroatoms may be nitrogen atoms, oxygen atoms or sulphur atoms.

A monocyclic heterocyclyl ring according to the present invention may have 3 to 8, preferably 4 to 7, particularly preferably 5 or 6 ring atoms.

By way of example and with preference, the following may be mentioned for monocyclic heterocyclyl radicals having 3 ring atoms:

aziridinyl-.

By way of example and with preference, the following may be mentioned for monocyclic heterocyclyl radicals having 4 ring atoms:

azetidinyl-, oxetanyl-.

By way of example and with preference, the following may be mentioned for monocyclic heterocyclyl radicals having 5 ring atoms:

pyrrolidinyl-, imidazolidinyl-, pyrazolidinyl-, pyrrolinyl-, dioxolanyl- and tetrahydrofuranyl-.

By way of example and with preference, the following may be mentioned for monocyclic heterocyclyl radicals having 6 ring atoms:

piperidinyl-, piperazinyl-, morpholinyl-, dioxanyl-, tetrahydropyranyl- and thiomorpholinyl-.

By way of example and with preference, the following may be mentioned for monocyclic heterocyclyl radicals having 7 ring atoms:

azepanyl-, oxepanyl-, 1,3-diazepanyl-, 1,4-diazepanyl-.

By way of example and with preference, the following may be mentioned for monocyclic heterocyclyl radicals having 8 ring atoms:

oxocanyl-, azocanyl-.

From among the monocyclic heterocyclyl radicals, preference is given to 4- to 7-membered saturated heterocyclyl radicals having up to two heteroatoms from the group consisting of O, N and S.

Particular preference is given to morpholinyl-, piperidinyl- and pyrrolidinyl-.

### Spirocycloalkyl and Heterospirocycloalkyl

C₅-C₁₂-Spirocycloalkyl or C₅-C₁₂-heterospirocycloalkyl where one, two, three or four carbon atoms are replaced by heteroatoms as defined above in any combination is understood to mean a fusion of two saturated ring systems which share one common atom. Examples are spiro[2.2]pentyl, spiro[2.3]hexyl, azaspiro[2.3]hexyl, spiro[3.3]heptyl, azaspiro[3.3]heptyl, oxaazaspiro[3.3]heptyl, thiaazaspiro[3.3]heptyl, oxaspiro[3.3]heptyl, oxazaspiro[3.5]nonyl, oxazaspiro[3.4]octyl, oxazaspiro[5.5]undecyl, diazaspiro[3.3]heptyl, thiazaspiro[3.3]heptyl, thiazaspiro[3.4]octyl, azaspiro[5.5]decyl, and the further homologous spiro[3.4], spiro[4.4], spiro[5.5], spiro[6.6], spiro[2.4], spiro[2.5], spiro[2.6], spiro[3.5], spiro[3.6], spiro[4.5], spiro[4.6] and spiro[5.6] systems including the variants modified by heteroatoms as per the definition. Preference is given to C₆-C₁₀-heterospirocycloalkyl-, by way of example and with particular preference 2-azaspiro[3.3]heptyl-, 1-thia-6-azaspiro[3.3]heptyl-, 2-thia-6-azaspiro[3.3]heptyl-, 2-oxa-6-azaspiro[3.3]heptyl-, 2,6-diazaspiro[3.3]heptyl-, 2-oxa-6-azaspiro[3.4]octyl-, 2-oxa-6-azaspiro[3.5]nonyl-, 2-oxa-7-azaspiro[3.5]nonyl-, 8-azaspiro[4.5]decyl-, 2,8-diazaspiro[4.5]decyl-, 3-oxa-1,8-diazaspiro[4.5]decyl-.

### Bicycloalkyl and Heterobicycloalkyl

C₆-C₁₂-Bicycloalkyl or C₆-C₁₂-heterobicycloalkyl where one, two, three or four carbon atoms are replaced by heteroatoms as defined above in any combination is understood to mean a fusion of two saturated ring systems which share two directly adjacent atoms. Examples are radicals derived from bicyclo[2.2.0]hexyl-, bicyclo[3.3.0]octyl-, bicyclo[4.4.0]decyl-, bicyclo[5.4.0]undecyl-, bicyclo[3.2.0]heptyl-, bicyclo[4.2.0]octyl-, bicyclo[5.2.0]nonyl-, bicyclo[6.2.0]decyl-, bicyclo[4.3.0]nonyl-, bicyclo[5.3.0]decyl-, bicyclo[6.3.0]undecyl- and bicyclo[5.4.0]undecyl-, including the variants modified by heteroatoms, for example azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl- or azabicyclo[4.4.0]decyl-, and the further possible combinations as per the definition. Preference is given to C₆-C₁₀-heterobicycloalkyl-, by way of example and with particular preference perhydrocyclopenta[c]pyrrolyl-, perhydrofuro[3,2-c]pyridinyl-, perhydropyrrolo[1,2-a]pyrazinyl-, perhydropyrrolo[3,4-c]pyrrolyl-.

Preferred examples of C₆-C₁₂-bicycloalkyl- are perhydronaphthalenyl- (decalinyl-), perhydrobenzoannulenyl-, perhydroazulenyl-, perhydroindanyl-, perhydropentalenyl-.

### Bridged Cycloalkyl and Bridged Heterocycloalkyl

A bridged C₆-C₁₂ ring system such as bridged C₆-C₁₂-cycloalkyl- or bridged C₆-C₁₂-heterocycloalkyl- is understood to mean a fusion of at least two saturated rings which share two atoms that are not directly adjacent to one another. This may give rise either to a bridged carbocycle (bridged cycloalkyl-) or to a bridged heterocycle (bridged heterocycloalkyl-) where one, two, three or four carbon atoms are replaced by heteroatoms as defined above in any combination. Examples are bicyclo[2.2.1]heptyl-, azabicyclo[2.2.1]heptyl-, oxazabicyclo[2.2.1]heptyl-, thiazabicyclo[2.2.1]heptyl-, diazabicyclo[2.2.1]heptyl-, bicyclo[2.2.2]octyl-, azabicyclo[2.2.2]octyl-, diazabicyclo[2.2.2]octyl-, oxazabicyclo[2.2.2]octyl-, thiazabicyclo[2.2.2]octyl-, bicyclo[3.2.1]octyl-, azabicyclo[3.2.1]octyl-, diazabicyclo[3.2.1]octyl-, oxazabicyclo[3.2.1]octyl-, thiazabicyclo[3.2.1]octyl-, bicyclo[3.3.1]nonyl-, azabicyclo[3.3.1]nonyl-, diazabicyclo[3.3.1]nonyl-oxazabicyclo[3.3.1]nonyl-, thiazabicyclo[3.3.1]nonyl-, bicyclo[4.2.1]nonyl-, azabicyclo[4.2.1]nonyl-, diazabicyclo[4.2.1]nonyl-, oxazabicyclo[4.2.1]nonyl-, thiazabicyclo[4.2.1]nonyl-, bicyclo[3.3.2]decyl-, azabicyclo[3.3.2]decyl-, diazabicyclo[3.3.2]decyl-, oxazabicyclo[3.3.2]decyl-, thiazabicyclo[3.3.2]decyl- or azabicyclo[4.2.2]decyl- and the further possible combinations according to the definition. Preference is given to bridged C₆-C₁₀-heterocycloalkyl-, by way of example and with particular preference 2-azabicyclo[2.2.1]heptyl-, 2,5-diazabicyclo[2.2.1]heptyl-, 2-oxa-5-azabicyclo[2.2.1]heptyl-, 8-azabicyclo[3.2.1]octyl-, 8-oxa-3-azabicyclo[3.2.1]octyl-, 3,9-diazabicyclo[4.2.1]nonyl-.

### Halogen

The term “halogen” or “halo” includes fluorine, chlorine, bromine and iodine.

Preference is given to fluorine and chlorine.

### Haloalkyl

Haloalkyl represents an alkyl radical having at least one halogen substituent.

A halo-C₁-C₆-alkyl radical is an alkyl radical having 1-6 carbon atoms and at least one halogen substituent. If a plurality of halogen substituents is present, these may also be different from one another. Preference is given to fluoro-C₁-C₆-alkyl, fluoro-C₁-C₄-alkyl and fluoro-C₁-C₃-alkyl radicals.

Examples which may be mentioned as being likewise preferred are:

the trifluoromethyl, 2,2,2-trifluoroethyl, pentafluoroethyl, 4,4,5,5,5-pentafluoropentyl or 3,3,4,4,5,5,5-heptafluoropentyl group.

Preference is given to perfluorinated alkyl radicals such as trifluoromethyl or pentafluoroethyl.

### Haloalkoxy

Haloalkoxy represents an alkoxy radical having at least one halogen substituent.

A halo-C₁-C₆-alkoxy radical is an alkoxy radical having 1-6 carbon atoms and at least one halogen substituent. If a plurality of halogen substituents is present, these may also be different from one another. Preference is given to fluoro-C₁-C₆-alkoxy, fluoro-C₁-C₄-alkoxy and fluoro-C₁-C₃-alkoxy radicals.

Examples which may be mentioned as being likewise preferred are:

the trifluoromethoxy or 2,2,2-trifluoroethoxy radical.

### Hydroxyalkyl

Haloalkyl represents an alkyl radical having at least one hydroxy substituent.

A hydroxy-C₁-C₆-alkyl radical is an alkyl radical consisting of 1-6 carbon atoms and at least one hydroxy substituent.

### Aminoalkyl

Aminoalkyl represents an alkyl radical having at least one amino substituent.

An amino-C₁-C₆-alkyl radical is an alkyl radical consisting of 1-6 carbon atoms and at least one amino substituent.

### Alkylaminoalkyl

Alkylaminoalkyl- represents an alkyl radical substituted by alkylamino as defined above, for example C₁-C₆-alkylamino-C₁-C₆-alkyl- or C₁-C₃-alkylamino-C₁-C₃-alkyl-.

Here, C₁-C₆-alkylamino-C₁-C₆-alkyl- means that the alkylaminoalkyl group is attached via the alkyl moiety to the remainder of the molecule.

Dialkylaminoalkyl-, for example di-C₁-C₃-alkylamino-C₁-C₃-alkyl-, means, that the alkylamino moiety mentioned above obligatorily contains two alkyl groups which may be identical or different. Examples of alkylaminoalkyl are N,N-dimethylaminoethyl-, N,N-dimethylaminomethyl-, N,N-diethylaminoethyl-, N,N-dimethylaminopropyl-, N-methylaminoethyl-, N-methylaminomethyl-.

Compounds according to the invention are the compounds of the formula (I) and their salts, solvates and solvates of the salts, the compounds encompassed by formula (I) of the formulae mentioned below and their salts, solvates and solvates of the salts and the compounds encompassed by formula (I) and mentioned below as working examples, and their salts, solvates and solvates of the salts, if the compounds encompassed by formula (I) and mentioned below are not already salts, solvates and solvates of the salts.

The present invention is likewise considered to encompass the use of the salts of the compounds according to the invention.

Preferred salts in the context of the present invention are physiologically acceptable salts of the compounds according to the invention. The invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention furthermore include base addition salts, for example of alkali metals such as sodium and potassium, of alkaline earth metals such as calcium and magnesium, or of ammonium salts derived from ammonia or organic amines having 1 to 16 carbon atoms, for example methylamine, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base or 1-amino-2,3,4-butanetriol. Furthermore, the compounds according to the invention may form base addition salts with quarterary ammonium ions which can be obtained, for example, by quarternization of corresponding amines with agents such as lower alkyl halides, for example methyl-, ethyl-, propyl- and butyl chlorides, bromides and iodides, dialkyl sulphates such as dimethyl, diethyl, dibutyl and diamyl sulphate, long-chain halides such as decyl, lauryl, myristyl and stearyl chlorides, bromides and iodides, or arylalkyl halides such as benzyl bromide or phenethyl bromide. Examples of such quarternary ammonium ions are tetramethylammonium, tetraethylammonium, tetra(n-propyl)ammonium, tetra(n-butyl)ammonium and also benzyltrimethylammonium.

The present invention further provides all the possible crystalline and polymorphous forms of the compounds according to the invention, where the polymorphs may be present either as single polymorphs or as a mixture of a plurality of polymorphs in all concentration ranges.

The present invention furthermore provides medicaments comprising the compounds according to the invention and at least one or more further active compounds, in particular for the prophylaxis and/or therapy of neoplastic disorders.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else optionally as conformational isomers. The compounds according to the invention may have a centre of asymmetry at the carbon atom to which R² is attached (C-4). They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses diastereomers and the respective mixtures thereof. The pure enantiomers and diastereomers can be isolated from the mixtures mentioned in a known manner; chromatography processes are preferably used for this, in particular HPLC chromatography on a chiral or achiral phase.

In general, the stereoisomers according to the invention inhibit the target to different degrees and have different activity in the cancer cell lines studied. The more active stereoisomer is preferred, which is often that in which the centre of asymmetry represented by the carbon atom bonded to R² has (S) configuration.

The present invention further provides stereoisomer mixtures of the (4S)-configured compounds according to the invention with their (4R) isomers, especially the corresponding racemates, diastereomer and enantiomer mixtures in which the (4S) form predominates.

Where the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here as meaning a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example to an extension of the half-life in the body or to a reduction in the active dose required; such modifications of the compounds according to the invention may therefore in some cases also constitute a preferred embodiment of the present invention. Isotopic variants of the compounds according to the invention can be prepared by generally customary processes known to those skilled in the art, for example by the methods described below and the procedures reported in the working examples, by using corresponding isotopic modifications of the particular reagents and/or starting compounds therein.

In addition, the present invention also encompasses prodrugs of the compounds according to the invention. The term “prodrugs” includes compounds which may themselves be biologically active or inactive but are converted to compounds according to the invention while resident in the body (for example metabolically or hydrolytically).

The compounds according to the invention can act systemically and locally. For this purpose, they can be administered in a suitable manner, for example by the oral, parenteral, pulmonary, nasal, sublingual, lingual, buccal, rectal, dermal, transdermal, conjunctival or otic route, or as implant or stent.

The compounds according to the invention can be administered in suitable administration forms for these administration routes.

Suitable administration forms for oral administration are those which function according to the prior art and deliver the compounds according to the invention rapidly and in modified fashion, and which contain the compounds according to the invention in crystalline or amorphized or dissolved form, for example tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and odour correctants.

The present invention further provides medicaments comprising the compounds according to the invention, typically together with one or more inert, nontoxic, pharmaceutically suitable excipients, and for the use thereof for the aforementioned purposes.

The compounds according to the invention are formulated to give pharmaceutical preparations in a manner known per se, by converting the active compound(s) to the desired administration form with the excipients customary in pharmaceutical formulation.

The excipients used may, for example, be carrier substances, fillers, disintegrants, binders, humectants, glidants, absorbents and adsorbents, diluents, solvents, cosolvents, emulsifiers, solubilizers, taste correctors, colourants, preservatives, stabilizers, wetting agents, salts for modifying the osmotic pressure or buffers. Reference should be made to Remington's Pharmaceutical Science, 15th ed. Mack Publishing Company, East Pennsylvania (1980).

The pharmaceutical formulations may be

in solid form, for example in the form of tablets, coated tablets, pills, suppositories, capsules, transdermal systems, or
in semisolid form, for example in the form of ointments, creams, gels, suppositories, emulsions, or
in liquid form, for example in the form of solutions, tinctures, suspensions or emulsions.

Excipients in the context of the invention may, for example, be salts, saccharides (mono-, di-, tri-, oligo- and/or polysaccharides), proteins, amino acids, peptides, fats, waxes, oils, hydrocarbons and derivatives thereof, and the excipients may be of natural origin or be obtained by synthetic or partially synthetic means.

Useful forms for oral or peroral administration are especially tablets, coated tablets, capsules, pills, powders, granules, pastilles, suspensions, emulsions or solutions.

Useful forms for parenteral administration are especially suspensions, emulsions, and particularly solutions.

The present invention relates to the compounds according to the invention.

They can be used for the prophylaxis and therapy of human disorders, in particular neoplastic disorders.

The compounds according to the invention can be used in particular for inhibiting or reducing cell proliferation and/or cell division and/or to induce apoptosis.

The compounds according to the invention are suitable in particular for the prophylaxis and therapy of hyper-proliferative disorders such as, for example,
psoriasis,
keloids and other skin hyperplasias,
benign prostate hyperplasias (BPH),
solid tumours and
haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours which can be treated are, for example,
multiple myelomas
lymphomas or
leukaemias

Breast tumours which can be treated are, for example:
breast carcinomas with positive hormone receptor status
breast carcinomas with negative hormone receptor status
Her-2 positive breast carcinomas
hormone receptor and Her-2 negative breast carcinomas
BRCA-associated breast carcinomas
inflammatory breast carcinomas.

Tumours of the respiratory tract which can be treated are, for example,
non-small-cell bronchial carcinomas such as, for example, squamous cell carcinoma, adenocarcinoma, large-cell carcinoma and
small-cell bronchial carcinomas.

Tumours of the brain which can be treated are, for example,
gliomas,
glioblastomas,
astrocytomas,
meningiomas and
medulloblastomas.

Tumours of the male reproductive organs which can be treated are, for example:
prostate carcinomas,
malignant epididymal tumours
malignant testicular tumours and
penis carcinomas.

Tumours of the female reproductive organs which can be treated are, for example:
endometrial carcinomas
cervix carcinomas
ovarian carcinomas
vaginal carcinomas
vulvar carcinomas

Tumours of the gastrointestinal tract which can be treated are, for example:
colorectal carcinomas
anal carcinomas
stomach carcinomas
pancreas carcinomas
oesophagus carcinomas
gall bladder carcinomas
carcinomas of the small intestine
salivary gland carcinomas
neuroendocrine tumours
gastrointestinal stroma tumours

Tumours of the uorgenital tract which can be treated are, for example:
urinary bladder carcinomas
kidney cell carcinomas
carcinomas of the renal pelvis and lower urinary tract

Tumours of the eye which can be treated are, for example:
retinoblastomas
intraocular melanomas

Tumours of the liver which can be treated are, for example:
hepatocellular carcinomas
cholangiocellular carcinomas

Tumours of the skin which can be treated are, for example:
malignant melanomas
basaliomas
spinaliomas
Kaposi sarcomas
Merkel cell carcinomas

Tumours of the head and neck which can be treated are, for example:
larynx carcinomas
carcinomas of the pharynx and the oral cavity
carcinomas of the middle line structures (e.g. NMC, C. A. French, Annu. Rev. Pathol. 2012, 7:247-265)

Sarcomas which can be treated are, for example:
soft tissue sarcomas
osteosarcomas

Lymphomas which can be treated are, for example:
non-Hodgkin lymphomas
Hodgkin lymphomas
cutaneous lymphomas
lymphomas of the central nervous system
AIDS-associated lymphomas

Leukaemias which can be treated are, for example:
acute myeloid leukaemias
chronic myeloid leukaemias
acute lymphatic leukaemias
chronic lymphatic leukaemias
hairy cell leukaemias

Advantageously, the compounds according to the invention can be used for prophylaxis and/or treatment of leukaemias, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

Particularly advantageously, the compounds according to the invention can be used for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The compounds according to the invention are also suitable for prophylaxis and/or treatment of benign hyperproliferative diseases, for example endometriosis, leiomyoma and benign prostate hyperplasia.

The compounds according to the invention are also suitable for male fertility control.

The compounds according to the invention are also suitable for prophylaxis and/or treatment of systemic inflammatory diseases, especially LPS-induced endotoxic shock and/or bacteria-induced sepsis.

The compounds according to the invention are also suitable for prophylaxis and treatment of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease,
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritides; collagenoses of any origin, e.g. systemic lupus erythematosus, scleroderma, polymyositis, dermatomyositis, Sjögren's syndrome, Still's syndrome, Felty's syndrome
allergies associated with inflammatory and/or proliferative processes: all forms of allergic reactions, e.g. angiooedema, hay fever, insect bites, allergic reactions to medicaments, blood derivatives, contrast agents, etc., anaphylactic shock, urticaria, contact dermatitis
vascular inflammation (vasculitis): panarteritis nodosa, temporal arteritis, erythema nodosum
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma,
renal disorders associated with inflammatory, allergic and/or proliferative processes: nephrotic syndrome; all nephritides,
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue
proctological disorders associated with inflammatory, allergic and/or proliferative processes: anal eczema; fissures; haemorrhoids; idiopathic proctitis,
ocular disorders associated with inflammatory, allergic and/or proliferative processes: allergic keratitis, uveitis, iritis; conjunctivitis; blepharitis; optic neuritis; chlorioditis; sympathetic ophthalmia
disorders of the ear-nose-throat region associated with inflammatory, allergic and/or proliferative processes: allergic rhinitis, hay fever; otitis externa, for example caused by contact eczema, infection, etc.; otitis media
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome
haematological disorders associated with inflammatory, allergic and/or proliferative processes: congenital haemolytic anaemia; idiopathic thrombocytopenia,
neoplastic disorders associated with inflammatory, allergic and/or proliferative processes: acute lymphatic leukaemia; malignant lymphoma; lymphogranulomatoses;
lymphosarcoma; extensive metastases, particularly in the case of mammary, bronchial and prostate carcinoma
endocrine disorders associated with inflammatory, allergic and/or proliferative processes: endocrine orbitopathy; thyrotoxic crisis; de Quervain's thyroiditis; Hashimoto's thyroiditis; Basedow's disease,
organ and tissue transplants, graft-versus-host disease,
severe states of shock, for example anaphylactic shock, systemic inflammatory response syndrome (SIRS)
substitution therapy in the case of: congenital primary renal insufficiency, for example congenital adrenogenital syndrome; acquired primary renal insufficiency, for example Addison's disease, autoimmune adrenalitis, postinfectious tumours, metastases, etc; congenital secondary renal insufficiency, for example congenital hypopituitarism; acquired secondary renal insufficiency, for example postinfectious, tumours, etc.
emesis associated with inflammatory, allergic and/or proliferative processes, for example in combination with a 5-HT3 antagonist in the case of cytostatic-induced vomiting
pain of inflammatory origin, for example lumbago.

The compounds according to the invention are also suitable for the treatment of viral disorders, for example infections caused by papilloma viruses, herpes viruses, Epstein-Barr viruses, hepatitis B or C viruses, and human immunodeficiency viruses.

The compounds according to the invention are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The compounds according to the invention are also suitable for the treatment of neurodegenerative diseases, for example multiple sclerosis, Alzheimer's disease and Parkinson's disease.

These disorders are well characterized in man, but also exist in other mammals.

The present application further provides the compounds according to the invention for use as medicaments, especially for prophylaxis and/or treatment of neoplastic disorders.

The present application further provides the compounds according to the invention for prophylaxis and treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides the compounds according to the invention for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the compounds according to the invention for production of a medicament.

The present application further provides for the use of the compounds according to the invention for production of a medicament for prophylaxis and treatment of neoplastic disorders.

The present application further provides for the use of the compounds according to the invention for production of a medicament for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the compounds according to the invention for production of a medicament for prophylaxis and treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides for the use of the compounds according to the invention for prophylaxis and treatment of neoplastic disorders.

The present application further provides for the use of the compounds according to the invention for prophylaxis and treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the compounds according to the invention for prophylaxis and treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides pharmaceutical formulations in the form of tablets comprising one of the compounds according to the invention for prophylaxis and treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides pharmaceutical formulations in the form of tablets comprising one of the compounds according to the invention for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the compounds according to the invention for treatment of disorders associated with proliferative processes.

The invention further provides for the use of the compounds according to the invention for treatment of benign hyperplasias, inflammation disorders, autoimmune disorders, sepsis, viral infections, vascular disorders and neurodegenerative disorders.

The compounds according to the invention can be used alone or, if required, in combination with one or more other pharmacologically active substances, provided that this combination does not lead to undesirable and unacceptable side effects. Accordingly, the present invention further provides medicaments comprising a compound according to the invention and one or more further active compounds, in particular for the prophylaxis and/or therapy of the disorders mentioned above.

For example, the compounds according to the invention can be combined with known antihyperproliferative, cytostatic or cytotoxic substances for treatment of cancer. The combination of the compounds according to the invention with other substances commonly used for cancer treatment, or else with radiotherapy, is particularly appropriate.

An illustrative but nonexhaustive list of suitable combination active compounds is as follows: abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, copanlisib, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, farston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotin, formestane, fosteabine, fotemustine, fulvestrant, Gammagard, gefitinib, gemcitabine, gemtuzumab, Gleevec, Gliadel, goserelin, gossypol, granisetrone hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lenalidomide, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, Osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, tastolactone, Taxoprexin, Taxoter, teceleukin, temozolomide, temsirolimus, teniposide, testosterone propionate, Testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, tastuzumab, teosulfan, transMID-107R, tretinoin, Trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflumine, vinorelbine, virulizin, vismodegib, Xeloda, Z-100, Zinecard, zinostatin stimalamer, zofran, zoledronic acid.

The combination of the compound according to the invention with a P-TEFb or CDK9 inhibitor is likewise particularly preferred.

In a promising manner, the compounds according to the invention can also be combined with bio-logics such as antibodies (for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab, trastuzumab) and recombinant proteins.

The compounds according to the invention can also achieve positive effects in combination with other therapies directed against angiogenesis, for example with bevacizumab, axitinib, regorafenib, cediranib, sorafenib, sunitinib or thalidomide. By virtue of their favourable side-effect profile, combinations with antihormones and steroidal metabolic enzyme inhibitors are particularly suitable.

Generally, the following aims can be pursued with the combination of the compounds according to the invention with other cytostatically or cytotoxically active agents:
improved efficacy in slowing the growth of a tumour, in reducing its size or even in the complete elimination thereof, compared with treatment with an individual active compound;
the possibility of using the chemotherapeutics used in a lower dosage than in the case of monotherapy;
the possibility of a more tolerable therapy with fewer side effects compared with individual administration;
the possibility of treatment of a broader spectrum of tumours;
the achievement of a higher rate of response to the therapy;
a longer survival time of the patient compared with present-day standard therapy.

In addition, the compounds according to the invention can also be used in conjunction with radiotherapy and/or surgical intervention.

### Preparation of the Compounds of the General Formula (I) According to the Invention

### Synthesis Routes for Preparing the Compounds of the General Formula (I)

The following schemes and general procedures illustrate the general synthetic access to the compounds of the formula (I) according to the invention; however, this should not be interpreted as meaning that the synthesis of the compounds according to the invention is limited to these. 4,5-Dihydro-3H-2,3-benzodiazepines of the general formula (I) can be prepared analogously to processes described in the literature. Depending on the substituents present, protective group strategies may be required; however, these are generally known to the person skilled in the art. Scheme 1 shows the synthesis of 4,5-dihydro-3H-2,3-benzodiazepines using a 3,4-dihydro-1H-2-benzopyran intermediate (III), where A, n and the radicals R^{1a}, R^{1b}, R², R⁴ and R⁵ have the meanings given in the general formula (I). Corresponding approaches are described, for example, in F. Gatta et al. Il Farmaco—Ed. Sc. 1985, 40, 942 or in WO2008124075 or WO200198280.

The aldehydes (IIa) used are commercially available, or their preparation is known to the person skilled in the art. R^{1a} and R^{1b} can also be introduced at a later stage of the synthesis, for example as described in Scheme 5.

The 1-aryl-2-propanols (II) used are either commercially available or are prepared in a manner generally known to the person skilled in the art by reduction of the corresponding ketones (Ia), for example by reduction with lithium aluminium hydride in THF.

This synthesis route is preferably used for arylpropanols (II) having electron-rich substituents (e.g. with alkoxy).

3,4-Dihydro-1H-2-benzopyrans (III) are obtained by condensation of the 1-aryl-2-propanols (II) with aromatic or heteroaromatic aldehydes (IIa) under acidic conditions. The reaction is carried out at elevated temperature (about 100° C.) in dioxane saturated with HCl, in the presence of anhydrous zinc chloride. Further conversion of the 3,4-dihydro-1H-2-benzopyrans (III) can be by various routes:

oxidative ring-opening of the 3,4-dihydro-1H-2-benzopyrans (III) using chromium(VI) oxide/sulphuric acid affords the diketone (IV) which can be cyclized with hydrazine to give 4-methyl-1-aryl-5H-2,3-benzodiazepine or 4-methyl-1-heteroaryl-5H-2,3-benzodiazepine (V) (cf. U.S. Pat. No. 5,288,863). Reduction, for example with sodium cyanoborohydride (Synthetic Communications, 2002, 32, 527), then yields the desired 4,5-dihydro-3H-2,3-benzodiazepine derivative (VI). Oxidation of the 3,4-dihydro-1H-2-benzopyrans (III) with atmospheric oxygen affords the 1-aryl-3,4-dihydro-1H-2-benzopyran-1-ol or 1-heteroaryl-3,4-dihydro-1H-2-benzopyran-1-ol (VII) which, with elimination of water using H₂NNHBoc, can be converted into the corresponding hydrazone derivative (VIII). This can be cyclized, for example by mesylation and subsequent treatment with base, to give the Boc-protected 4,5-dihydro-3H-2,3-benzodiazepine derivative (IX), which in turn can be converted by acidic deprotection in a generally known manner into the corresponding 4,5-dihydro-3H-2,3-benzodiazepine derivative (VI).

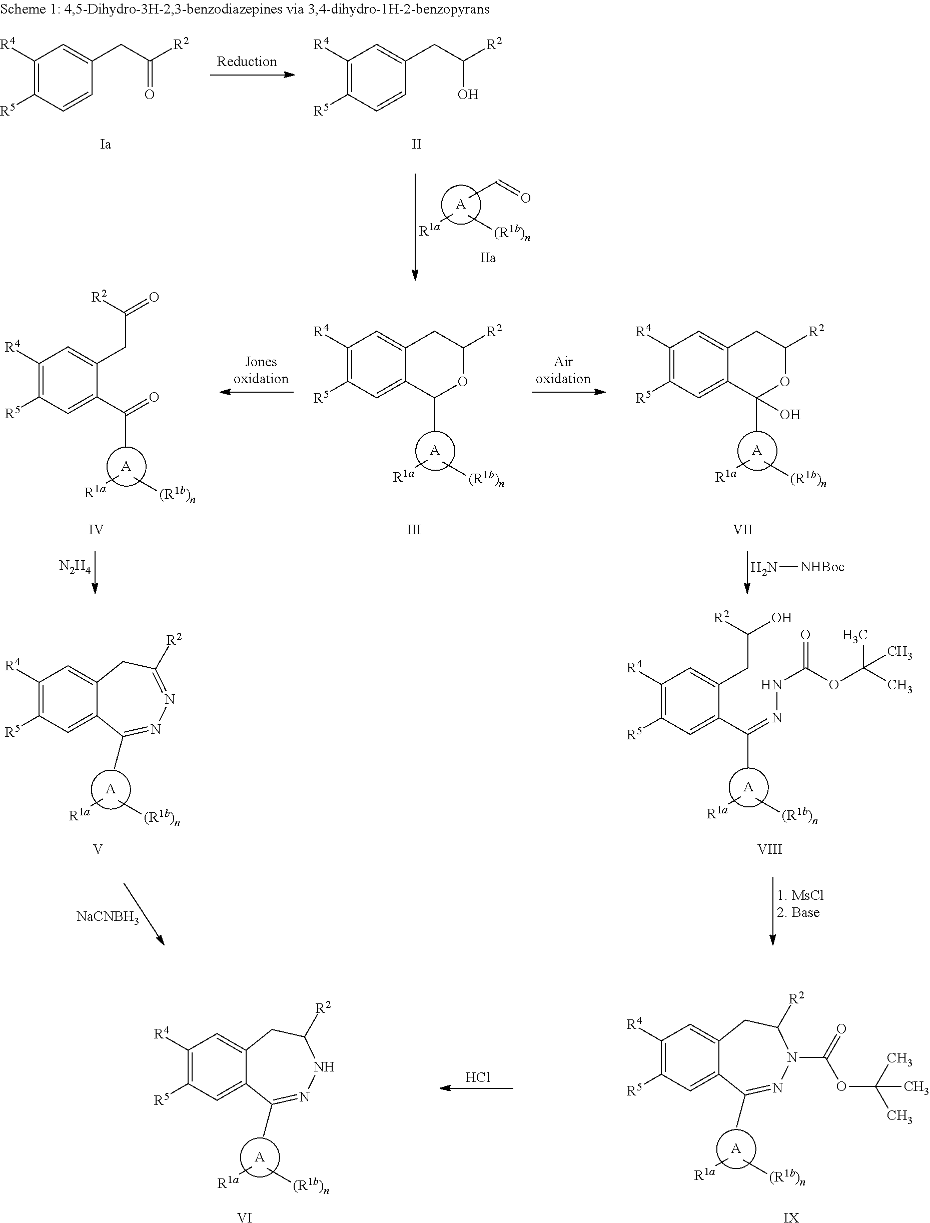

Scheme 2 describes the synthesis of 4,5-dihydro-3H-2,3-benzodiazepines from indanones (X).

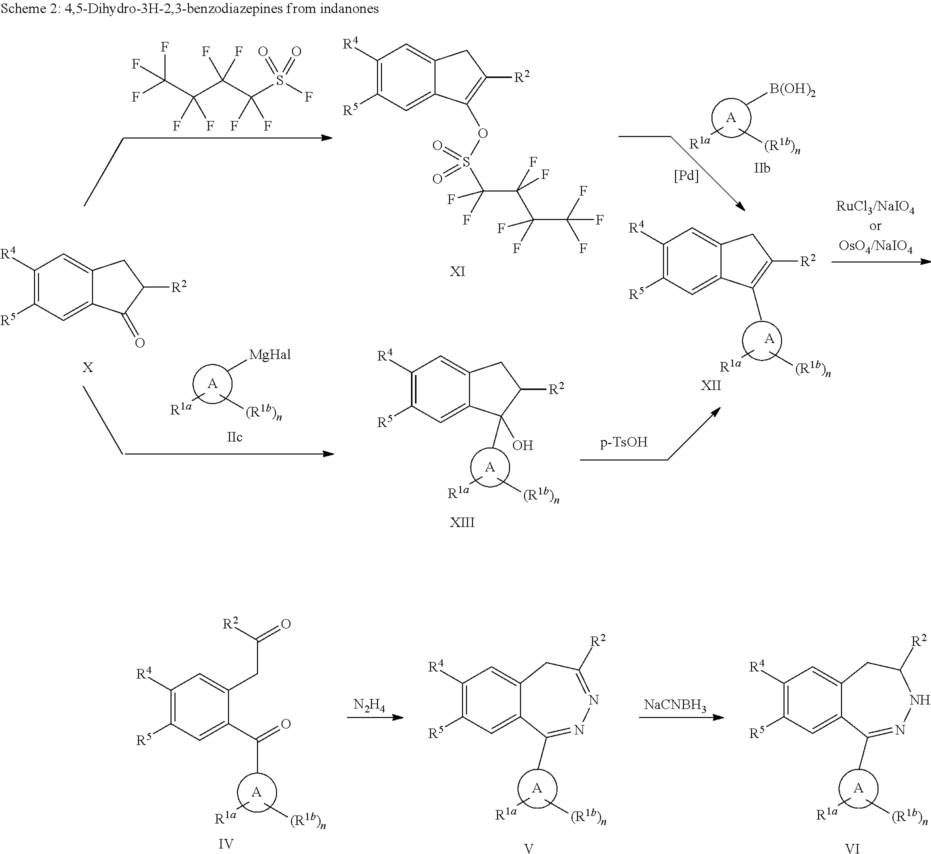

A, n and the radicals R^{1a}, R^{1b}, R², R⁴ and R⁵ in Scheme 2 have the meanings given in the general formula (I).

The indanone (X) can be converted into the corresponding 3-aryl-1H-indene or 3-heteroaryl-1H-indene (XII). To this end, the following processes may be used:
the indanone derivative (X) can, for example, be converted in a generally known manner into the corresponding enol nonaflate (XI) and then be converted by palladium-catalysed Suzuki coupling with the appropriate boronic acid derivatives (IIb) into the indene (XII).
the indanone derivative (X) can be converted by addition of organomagnesium reagents (IIc) in a generally known manner into the corresponding indanols (XIII) which, via acid-catalysed elimination, readily form the corresponding indenes (X).

The 3-aryl-1H-indenes or 3-heteroaryl-1H-indenes (XII) can be converted by oxidative methods using, for example, ruthenium(III) chloride/sodium periodate (Bioorganic and Medicinal Chemistry Letters, 2011, 21, 2554) into the corresponding diketones (IV). These can be converted analogously to Scheme 1 into the corresponding 4,5-dihydro-3H-2,3-benzodiazepine derivatives (VI).

The indanones (X) used for preparing the working examples are either commercially available or can be prepared as shown, for example, in Scheme 3, where the radicals R², R⁴ and R⁵ have the meaning given in the general formula (I).

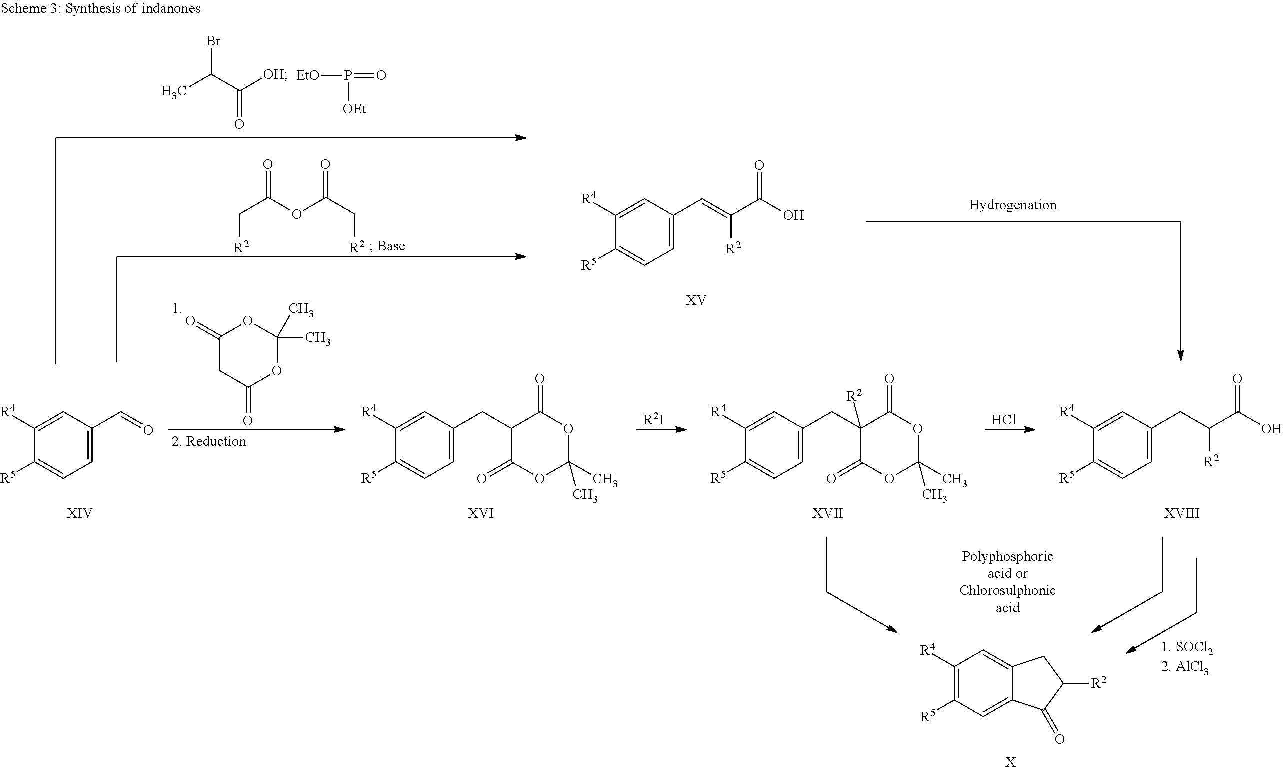

The 2-methyl-3-arylpropanoic acids (XVIII) can be prepared from the corresponding aromatic aldehydes (XIV) by processes known from the literature (cf. Angewandte Chemie, International Edition, 2012, 51, 1265). These can be cyclized using, for example, chlorosulphonic acid or polyphosphoric acid, giving the corresponding indanones (X) (cf. Synthesis 2009, 627 and Org. Process Res. Dev. 2011, 15, 570-580, J. Org. Chem. 2005, 70, 1316 and Bioorg. Med. Chem. Lett. 2011, 21, 2554-2558).

Scheme 4 illustrates the preparation of the exemplary compounds according to the invention starting with 4,5-dihydro-3H-2,3-benzodiazepines (VI) using generally known reactions, for example with acid chlorides, anhydrides, chloroformates or isocyanates or isothiocyanates, where A, n and the radicals R^{1a}, R^{1b}, R², R³, R⁴ and R⁵ have the meanings giving in general formula (I). The corresponding alkylureas (XIX) can also be obtained by reacting a reactive intermediate such as, for example, the 4-nitrophenyl carbamate, with alkylamines.

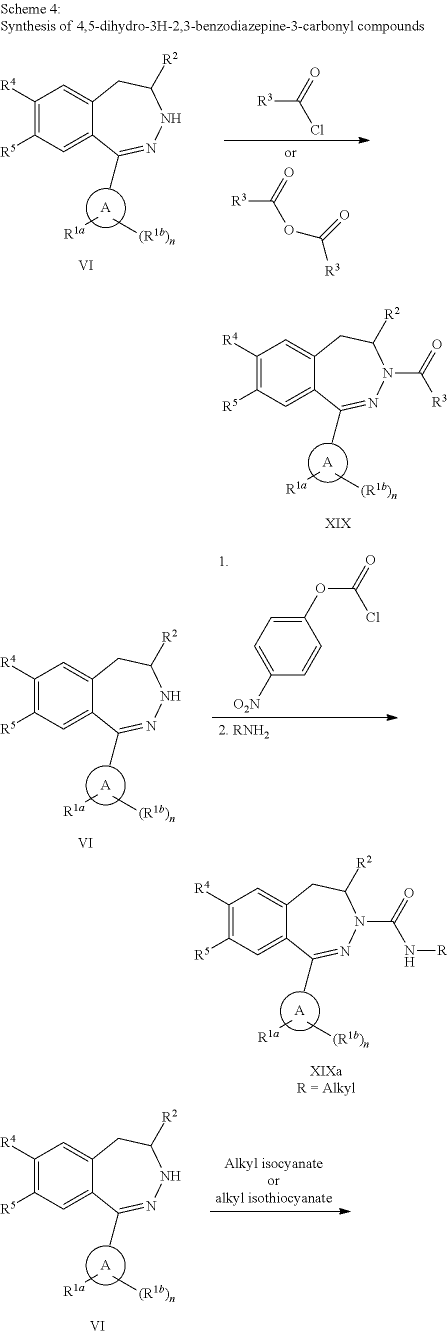

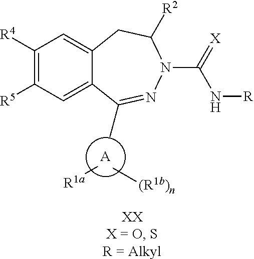

R^{1a}, R⁴ and R⁵ can also be introduced at a later stage of the synthesis, for example as described in Scheme 5.

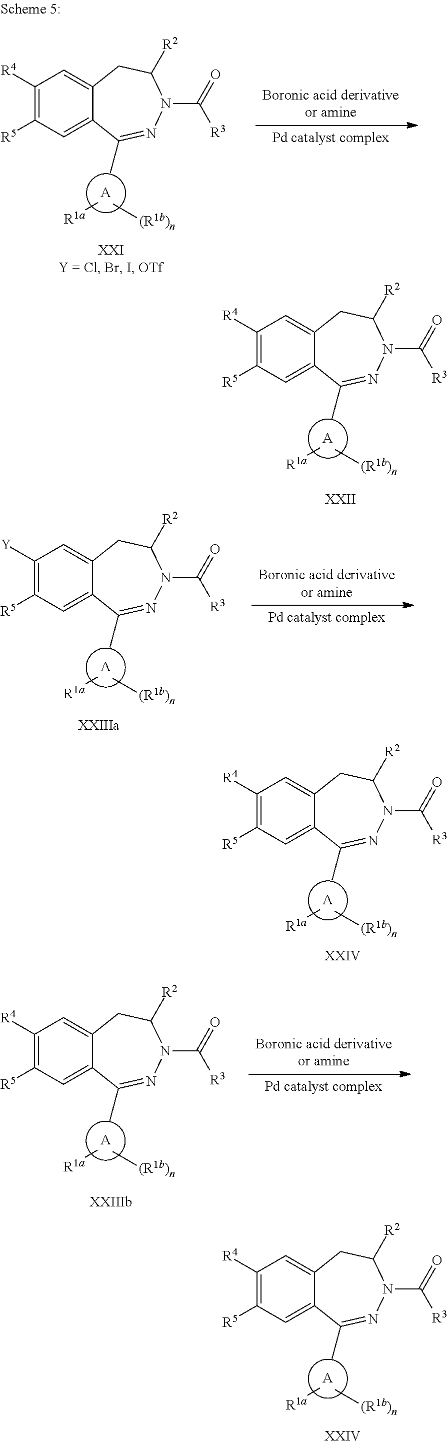

The radicals R^{1a}, R^{1b}, R², R³, R⁴, R⁵ and n in Scheme 5 have the meanings given in the general formula (I).

Scheme 5 illustrates the preparation of working examples which can be prepared by palladium-catalysed coupling reactions generally known to the person skilled in the art starting, for example, with bromine-substituted aryl- or heteroaryl derivatives (XXI, XXIIIa and XXIIIb) by reaction with the appropriate boronic acid derivatives (Chem. Rev. 1995, 95, 2457-2483; Angewandte Chemie, International Edition (2002), 41(22), 4176-4211) or amines. Intermediates XXI, XXIIIa and XXIIIb can be prepared analogously to the synthesis routes shown.

Boronic acid derivatives and amines are commercially available or can be prepared in a generally known manner. The preparation of the exemplary compounds according to the invention by reacting amines is carried out, for example, under Buchwald-Hartwig conditions (Journal of Organometallic Chemistry 1999, 576(1-2), 125-146, Angew. Chem. Int. Ed. 2008, 47, 6338-6361, Chem. Eur. J. 2010, 16, 1983-1991).

### ABBREVIATIONS

abs. absolute
ACN acetonitrile
FA formic acid
Boc tert-butoxycarbonyl
CDCl₃ deuterochloroform
CO₂ carbon dioxide
d day
DIPEA N,N-diisopropylethylamine
DMAP 4-N,N-dimethylaminopyridine
DMF dimethylformamide
DMSO dimethyl sulphoxide
of th. of theory
eq. equivalent
ESI electrospray ionization (in MS)
sat. saturated
h hour
HOBt 1-hydroxy-1H-benzotriazole×H₂O
HPLC high-pressure, high-performance liquid chromatography
conc. concentrated
LC-MS liquid chromatography-coupled mass spectrometry
min minutes
MS mass spectrometry
Ms methanesulphonyl
MW molecular weight [g/mol]
NMP N-methylpyrrolidone
NMR nuclear magnetic resonance spectroscopy
R_{f} retention index (in TLC)
RP-HPLC reversed phase HPLC
RT room temperature
Rₜ retention time (in HPLC)
SFC supercritical fluid chromatography
TFA trifluoroacetic acid
THF tetrahydrofuran

### LC-MS Methods:

Method 1: Instrument: Waters Acquity LCT; column: Phenomenex Kinetex C18, 50 mm×2.1 mm, 2.6μ; mobile phase A: water/0.05% FA, mobile phase B: ACN/0.05% FA; gradient: 0.0 min 98% A→0.2 min: 98% A→1.7 min: 10% A→1.9 min: 10% A→2 min: 98% A→2.5 min: 98% A; flow rate: 1.3 ml/min; column temperature: 60° C.; UV detection: 200-400 nm.

Method 2: Instrument: Waters Acquity Platform ZQ4000; column: Waters BEHC 18, 50 mm×2.1 mm, 1.7μ; mobile phase A: water/0.05% FA, mobile phase B: ACN/0.05% FA; gradient: 0.0 min 98% A 4→0.2 min: 98% A→1.7 min: 10% A→1.9 min: 10% A→2 min: 98% A→2.5 min: 98% A; flow rate: 1.3 ml/min; column temperature: 60° C.; UV detection: 200-400 nm.

Method 3: UPLC-SQD-HCOOH; instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Analytical HPLC Methods:

Method A: System: Waters: Alliance 2695, DAD 996; column: Chiralpak ID-3 3 μm 100×4.6 mm; mobile phase: hexane/IPA 50:50 (v/v)+0.1% DEA; flow rate: 1.0 ml/min; column temperature: 25° C.; detection: DAD 254 nm.

Method B: System: Agilent: 1260 AS, MWD, Aurora SFC module; column: Chiralpak IA 5 μm 100×4.6 mm; mobile phase: CO₂/methanol 8:2; flow rate: 4.0 ml/min; pressure (outlet): 100 bar; column temperature: 37.5° C.; detection: DAD 254 nm.

Method C: System: Agilent: 1260 AS, MWD, Aurora SFC module; column: Chiralpak IA 5 μm 100×4.6 mm; mobile phase: CO₂/methanol 7:3; flow rate: 4.0 ml/min; pressure (outlet): 100 bar; column temperature: 37.5° C.; detection: DAD 254 nm.

Method D: System: Agilent: 1260 AS, MWD, Aurora SFC module; column: Chiralpak ID 5 μm 100×4.6 mm; mobile phase: CO₂/ethanol 6:4; flow rate: 4.0 ml/min; pressure (outlet): 100 bar; column temperature: 37.5° C.; detection: DAD 254 nm.

Method E: System: Waters: Alliance 2695, DAD 996, ESA: Corona; column: Chiralpak IC-3 μm 100×4.6 mm; mobile phase: ethanol/methanol/diethylamine 50:50:0.1 (v/v/v); flow rate: 1.0 ml/min; column temperature: 25° C.; detection: DAD 254 nm.

Method F: System: Agilent SFC 1200; column: Chiralpak AZ-H 5μ 250×4.6 mm; mobile phase: CO₂/isopropanol 70:30 (v/v); flow rate: 3 ml/min; detection: DAD 210 nm

Method G: System: Waters: Alliance 2695, DAD 996, ESA: Corona; column: Chiralpak IA-3 μm 100×4.6 mm; mobile phase: hexane/2-propanol/diethylamine 70:30:0.1 (v/v/v); flow rate: 1.0 ml/min; column temperature: 25° C.; detection: DAD 254 nm.

Method H: System: Waters: Alliance 2695, DAD 996, ESA: Corona; column: Chiralpak ID-3 μm 100×4.6 mm; mobile phase: hexane/2-propanol/diethylamine 70:30:0.1 (v/v/v); flow rate: 1.0 ml/min; column temperature: 25° C.; detection: DAD 280 nm.

Method J: System: Agilent: 1260 AS, MWD, Aurora SFC module; column: Chiralpak ID 3 m 100×4.6 mm; mobile phase: CO₂/ethanol 65:35+0.2% vol. diethylamine; flow rate: 4.0 ml/min; pressure (outlet): 100 bar; column temperature: 37.5° C.; detection: DAD 254 nm.

Method K: System: Agilent: 1260 AS, MWD, Aurora SFC module; column: Chiralpak IC 3 μm 100×4.6 mm; mobile phase: CO₂/2-propanol 60:30+0.2% vol. diethylamine; flow rate: 4.0 ml/min; pressure (outlet): 100 bar; column temperature: 37.5° C.; detection: DAD 254 nm.

### Preparative HPLC Methods:

Method I: System: Agilent: Prep 1200, 2×Prep pump G1361A, DLA G2258A, MWD G1365D, Prep FC G1364B; column: Chiralpak ID 5 μm 250×20 mm; mobile phase: hexane/IPA 50:50 (v/v)+0.1% DEA; flow rate: 30 ml/min; temperature: RT; detection: UV 254 nm.

Method II: System: Sepiatec: Prep SFC 100; column: Chiralpak IA 5 am 250×20 mm; mobile phase: CO₂/methanol 8:2; flow rate: 80 ml/min; pressure (outlet): 150 bar; temperature: 40° C.; detection: UV 254 nm.

Method III: System: Agilent: Prep 1200, 2×Prep Pump, DLA, MWD, Prep FC; column: Chiralpak ID 5 μm 250×20 mm; mobile phase: hexane/2-propanol 70:30 (v/v)+0.1% DEA; flow rate: 40 ml/min; temperature: RT; detection: UV 280 nm.

Method IV: System: Agilent: Prep 1200, 2×Prep Pump, DLA, MWD, Prep FC; column: Chiralpak IC 5 μm 250×30 mm; mobile phase: ethanol/methanol/diethylamine 70:30:0.1 (v/v/v); flow rate: 30 ml/min; temperature: RT; detection: UV 280 nm.

Method V: System: Sepiatec: Prep SFC 100, Prep FC; column: Chiralpak ID 5 μm 250×30 mm; Eluent: CO₂/ethanol 6/4; flow rate: 80 ml/min; temperature: 40° C.; detection: UV 254 nm.

Method VI: System: Agilent: Prep 1200, 2×Prep Pump, DLA, MWD, Prep FC; column: Chiralpak IA 5 μm 250×30 mm; mobile phase: hexane/2-propanol/diethylamine 70:30:0.1 (v/v/v); flow rate: 20 ml/min; temperature: RT; detection: UV 254 nm.

Method VII: System: Agilent: Prep 1200, 2×Prep Pump, DLA, MWD, Prep FC; column: Chiralpak ID 5 μm 250×30 mm; mobile phase: hexane/2-propanol/diethylamine 70:30:0.1 (v/v/v); flow rate: 50 ml/min; temperature: RT; detection: UV 280 nm.

Method VIII: System: Sepiatec: Prep SFC 100; column: Chiralpak IC 5 μm 250×20 mm; mobile phase: CO₂/2-propanol/diethylamine 60:40:0.4 (v/v/v); flow rate: 80 ml/min; temperature: 40° C.; detection: UV 254 nm.

### PREPARATION OF THE INTERMEDIATES

### Example 1A

### 1-(3,4-Dimethoxyphenyl)propan-2-ol

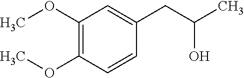

At 0° C., 147 mg (3.86 mmol) of lithium aluminium hydride were initially charged in 30 ml of THF, and 1.00 g (5.15 mmol) of 1-(3,4-dimethoxyphenyl)propan-2-one, dissolved in 10 ml of THF, were added dropwise. The mixture was stirred at 0° C. for 2 h, and 0.1 ml of water, 0.1 ml of 2M aqueous sodium hydroxide solution and a further 0.3 ml of water were then added carefully. After a further 30 min of stirring at RT, the mixture was filtered through silica gel/sodium sulphate, the filter cake was washed with ethyl acetate and the filtrate was concentrated on a rotary evaporator. This gave 950 mg of product (82% of theory) which was directly reacted further.

LCMS (method 2): Rₜ=0.82 min; m/z=197 (M+H)⁺; 179 (M−H₂O+H)⁺

¹H-NMR (300 MHz, DMSO-d₆): δ=0.98 (d, 3H), 2.43 (dd, 1H), 2.59 (dd, 1H), 3.67 (s, 3H), 3.69 (s, 3H), 3.70-3.79 (m, 1H), 4.43 (d, 1H), 6.65 (dd, 1H), 6.75 (d, 1H), 6.79 (d, 1H).

### Example 2A

### 1-(4-Bromophenyl)-3,4-dihydro-6,7-dimethoxy-3-methyl-1H-2-benzopyran

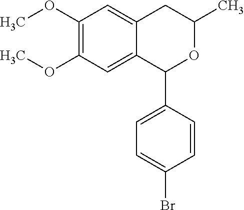

At RT, 960 mg (4.89 mmol) of 1-(3,4-dimethoxyphenyl)propan-2-ol (Example 1A) and 950 mg (5.14 mmol) of 4-bromobenzaldehyde (CAS [1122-91-4]) were initially charged in 4 ml of dioxane, and 7.70 ml of zinc chloride solution (0.7 M in THF, CAS [7646-85-7]) and 2.45 ml of hydrochloric acid (4 M in dioxane, CAS [7647-01-0]) were added. The mixture was then heated under reflux for 3 h and stirred at RT for a further 14 h. The mixture was added to water and extracted with ethyl acetate and the combined organic phases were washed with sat. sodium bicarbonate solution and sat. sodium chloride solution and dried with sodium sulphate. The solvents were removed on a rotary evaporator. This gave 3.0 g of crude product as a light-brown oil which was directly reacted further.

LCMS (Method 2): Rₜ=1.44 min; m/z=363; 365 (Br isotope pattern, M+H)⁺

Analogously to Example 2A, the following compounds were prepared from Example 1A and 3-bromobenzaldehyde or 3-bromo-4-fluorobenzaldehyde:

| No | Structure | Name | Analytical data |
|---|---|---|---|
| 3A | 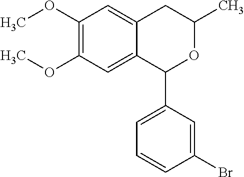 [SMILES: COc1cc2c(cc1OC)C(c1cccc(Br)c1)OC(C)C2] | 1-(3-bromophenyl)- 3,4-dihydro-6,7- dimethoxy-3-methyl- 1H-2-benzopyran | LCMS (method 3): Rₜ = 1.40 min; m/z = 363; 365 (M + H, Br isotope pattern)⁺ |
| 4A | 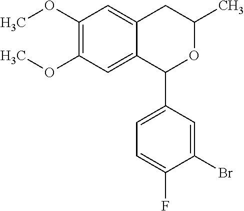 [SMILES: COc1cc2c(cc1OC)C(c1ccc(F)c(Br)c1)OC(C)C2] | 1-(3-bromo-4- fluorophenyl)-6,7- dimethoxy-3-methyl- 3,4-dihydro-1H-2- benzopyran | LCMS (method 3): Rₜ = 1.44 min; m/z = 381; 383 (Br isotope pattern, M + H)⁺ |

### Example 5A

### 1-[2-(4-Bromobenzoyl)-4,5-dimethoxyphenyl]propan-2-one

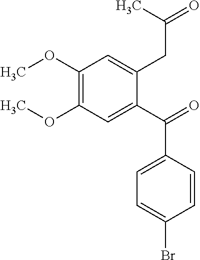 [SMILES: COc1cc(CC(C)=O)c(C(=O)c2ccc(Br)cc2)cc1OC]

At 0° C., 3.00 g (8.26 mmol) of 1-(4-bromophenyl)-3,4-dihydro-6,7-dimethoxy-3-methyl-1H-2-benzopyran (Example 2A) were initially charged together with 3 g of silica gel in 30 ml of acetone. A solution of 3.01 g (30.1 mmol) of chromium(VI) oxide (CAS [1333-82-0]) in 10 ml of conc. sulphuric acid and 20 ml of water was then slowly added dropwise, and the mixture was stirred at RT for 1 h. The red-brown mixture was then added to water and extracted with ethyl acetate. The organic phases were washed with sat. sodium chloride solution until neutral and dried with sodium sulphate and the solvents were removed on a rotary evaporator. The residue (3 g) was purified by flash chromatography (SiO₂, hexane/ethyl acetate). This gave 1.03 g (33% of theory, 2 steps) of the product as a yellow solid.

LCMS (Method 2): Rₜ=1.26 min; m/z=377; 379 (Br isotope pattern, M+H)⁺

Analogously to Example 5A, the following compounds were prepared from the corresponding 3,4-dihydro-1H-2-benzopyrans:

| No | Structure | Name | Analytical data |
|---|---|---|---|
| 6A | 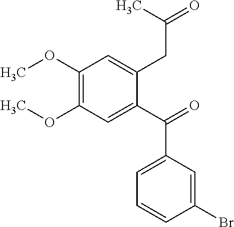 [SMILES: COc1cc(CC(C)=O)c(C(=O)c2cccc(Br)c2)cc1OC] | 1-[2-(3- Bromobenzoyl)-4,5- dimethoxyphenyl] propan-2-one | LCMS (method 3): Rₜ = 1.21 min; m/z = 377; 379 (M + H, Br isotope pattern)⁺ |
| 7A | 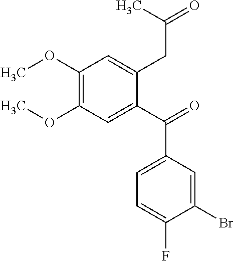 [SMILES: COc1cc(CC(C)=O)c(C(=O)c2ccc(F)c(Br)c2)cc1OC] | 1-[2-(3-bromo-4- fluorobenzoyl)-4,5- dimethoxyphenyl] propan-2-one | LCMS (method 3): Rₜ = 1.25 min; m/z = 395; 397 (Br isotope pattern, M + H)⁺ |

### Example 8A

### 2-Methyl-3-(4-nitrophenyl)acrylic acid

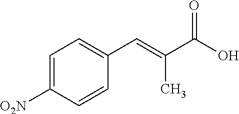

100 g (662 mmol) of 4-nitrobenzaldehyde, 114 g (1.19 mol) of sodium propionate (CAS [137-40-6]) and 86.1 g (662 mmol) of propionic anhydride (CAS [123-62-6]) were stirred together at 150° C. for 3 h. The warm mixture was diluted with water and cooled, and the precipitate formed was filtered off, washed with water and dried (vacuum drying cabinet, 40° C.). This gave 140 g of crude product as a pale yellow solid which was converted further without further purification.

LCMS (method 2): Rₜ=0.99 min; m/z [ES⁻]⁼206 (M−H)⁻

1H-NMR (400 MHz, DMSO-d₆): δ=2.01 (s, 3H), 7.62 (s, 1H), 7.70 (d, 2H), 8.23 (d, 2H), 12.8 (s, br, 1H).

### Example 9A

### (±)-3-(4-Aminophenyl)-2-methylpropanoic acid

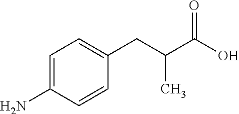

41.0 g (198 mmol) of 2-methyl-3-(4-nitrophenyl)acrylic acid (Example 8A) were dissolved in 380 ml of ethyl acetate, 4.21 g of palladium (10% on activated carbon) were added and the mixture was hydrogenated at atmospheric pressure with hydrogen for 3.5 h. This gave 32.0 g (90%) of the desired compound as a yellow oil which crystallizes.

LCMS (method 2): Rt=0.48 min; m/z=180 (M+H)+

1H-NMR (400 MHz, CDCl₃): δ=0.95 (d, 3H), 2.36 (dd, 1H), 2.42-2.45 (m, 1H), 2.70 (dd, 1H), 6.43 (d, 2H), 6.78 (d, 2H).

### Example 10A

### (±)-6-Amino-2-methylindan-1-one

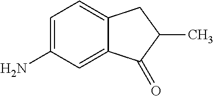

310 g of polyphosphoric acid were added to 38 g (11.1 mmol) of (±)-3-(4-aminophenyl)-2-methylpropanoic acid (obtained as described in Example 9A), and the mixture was stirred at 150° C. for 7 h using a compressed air stirrer. After cooling, the mixture was carefully diluted with water a little at a time and then, with ice cooling, made alkaline using 32% strength aqueous sodium hydroxide solution (pH=10). The mixture was extracted with dichloromethane and the combined organic phases were dried with sodium sulphate. The solvents were removed on a rotary evaporator and the crude product (26 g) was directly reacted further.

LCMS (method 2): Rₜ=0.69 min; m/z=162; 203 (M+H; M+ACN+H)⁺

¹H-NMR (300 MHz, DMSO-d₆): δ=1.11 (d, 3H), 2.53-2.60 (m, 1H), 2.68 (dd, 1H), 3.15 (dd, 1H), 5.25 (s, br, 2H), 6.71 (d, 1H), 6.88 (dd, 1H), 7.16 (d, 1H).

### Example 11A

### (±)-tert-Butyl (2-methyl-3-oxo-2,3-dihydro-1H-inden-5-yl)carbamate

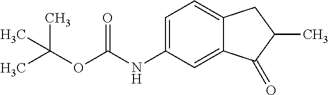 [SMILES: CC1Cc2ccc(NC(=O)OC(C)(C)C)cc2C1=O]

15.0 g (93.0 mmol) of (±)-6-amino-2-methylindan-1-one (Example 10A) were dissolved in 450 ml of dichloromethane and stirred in an ice bath for 10 min, 21.3 g (97.7 mmol) of di-tert-butyl dicarbonate were then added and the mixture was stirred at RT for a further 16 h. The mixture was added to water and extracted with dichloromethane, the combined organic phases were washed with sat. sodium chloride solution and the solvents were removed on a rotary evaporator. The crude product was purified chromatographically (SiO₂, hexane/ethyl acetate 0-30%). This gave 13.3 g (50% of theory) as a yellow foam.

LCMS (method 2): R=1.21 min; m/z=262; 303 (M+H)⁺; (M+ACN+H)⁺

¹H-NMR (400 MHz, DMSO-d₆): δ=1.14 (d, 3H), 1.45 (s, 9H), 2.58 (dd, 1H), 2.61-2.70 (m, 1H), 3.25 (dd, 1H), 7.40 (d, 1H), 7.63 (dd, 1H), 7.77 (d, 1H), 9.51 (s. 1H).

### Example 12A

### tert-Butyl [3-(4-chlorophenyl)-2-methyl-1H-inden-5-yl]carbamate

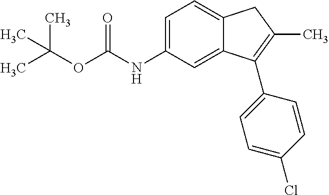 [SMILES: CC1=C(c2ccc(Cl)cc2)c2cc(NC(=O)OC(C)(C)C)ccc2C1]

Under argon, 124 ml of 4-chlorophenylmagnesium bromide (1M in diethyl ether, 124 mmol) were initially charged in 140 ml of THF, and 13.0 g (49.7 mmol) of (±)-tert-butyl (2-methyl-3-oxo-2,3-dihydro-1H-inden-5-yl)carbamate (Example 11A), dissolved in 60 ml of THF, were added dropwise at RT. The mixture was stirred at RT for 30 min and then added to sat. ammonium chloride solution and extracted 3× with ethyl acetate, the combined organic phases were washed with sat. sodium chloride solution and dried with sodium sulphate and the solvents were removed on a rotary evaporator.

The residue was taken up in 950 ml of dichloromethane, 142 g (750 μmol) of 4-toluenesulphonic acid monohydrate were added and the mixture was stirred at RT for 1 h. The reaction mixture was added to sat. sodium hydrogencarbonate solution and extracted 3× with dichloromethane, the combined organic phases were washed with sat. sodium chloride solution and dried with sodium sulphate and the solvent was removed on a rotary evaporator. The crude product (grey resin) was directly reacted further without further purification.

LCMS (method 2): Rₜ=1.64 min; m/z=256 (M+H)⁺

### Example 13A

### 2,2-Dimethyl-5-[4-(trifluoromethoxy)benzyl]-1,3-dioxane-4,6-dione

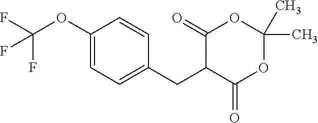 [SMILES: CC1(C)OC(=O)C(Cc2ccc(OC(F)(F)F)cc2)C(=O)O1]

25.4 g (134 mmol) of 4-(trifluoromethoxy)benzaldehyde (CAS [659-28-9]), 19.3 g (134 mmol) of Meldrum's acid (2,2-dimethyl-1,3-dioxane-4,6-dione, CAS [2033-24-1]) and 1.93 g (13.4 mmol) of piperidinium acetate (CAS [4540-33-4]) were dissolved in 500 ml of ethanol, and the mixture was stirred at RT for 30 min. The reaction solution was cooled to 0° C. using an ice bath and stirred for a further 10 min. 12.6 g (200 mmol) of sodium cyanoborohydride were introduced a little at a time and the mixture was allowed to warm to RT and stirred for a further 1.5 h. 250 ml of 2M hydrochloric acid were then added carefully and stirring was continued until the evolution of gas had ceased completely (about 30 min). The ethanol was removed on a rotary evaporator, the residue was taken up in 2M hydrochloric acid and the mixture was extracted repeatedly with dichloromethane. The combined organic phases were dried with sodium sulphate and the solvent was removed on a rotary evaporator. This gave 32.7 g (41% of theory) of crude product as a white solid which was converted further without further purification.

LCMS (method 1): Rₜ=1.33 min; m/z=319 (M+H)⁺

### Example 14A

### 2,2,5-Trimethyl-5-[4-(trifluoromethoxy)benzyl]-1,3-dioxane-4,6-dione

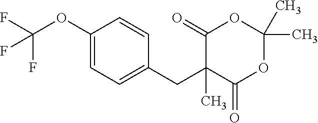 [SMILES: CC1(C)OC(=O)C(C)(Cc2ccc(OC(F)(F)F)cc2)C(=O)O1]

At RT, 32.7 g (103 mmol) of 2,2-dimethyl-5-[4-(trifluoromethoxy)benzyl]-1,3-dioxane-4,6-dione (Example 13A) and 21.3 g (154 mmol) of potassium carbonate were initially charged in 400 ml of DMF, and 72.9 g (514 mmol, 32.0 ml) of iodomethane were slowly added dropwise. The mixture was stirred vigorously at RT for 1.5 h and then added to water. The mixture was extracted 3× with ethyl acetate, the combined organic phases were washed with sat. sodium chloride solution and dried with sodium sulphate. The solvents were removed on a rotary evaporator and the crude product (32.5 g colourless oil) was purified by flash chromatography (SiO₂, hexane/ethyl acetate). This gave 20.0 mg (55% of theory) of the desired product as a colourless oil.

¹H-NMR (300 MHz, DMSO-d₆): δ=0.99 (s, 3H), 1.57 (s, 3H), 1.63 (s, 3H), 3.22 (s, 2H), 7.12 (d, 2H), 7.31 (s, 2H).

### Example 15A

### 2-Methyl-3-[4-(trifluoromethoxy)phenyl]propanoic acid

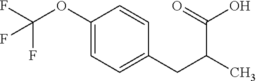 [SMILES: CC(Cc1ccc(OC(F)(F)F)cc1)C(=O)O]

19.0 g (57.2 mmol) of 2,2,5-trimethyl-5-[4-(trifluoromethoxy)benzyl]-1,3-dioxane-4,6-dione (Example 14A) were taken up in 90 ml of dioxane and 35 ml of conc. aqueous hydrochloric acid and heated under reflux at 125° C. for 2 h. The mixture was allowed to cool and the solvents were removed on a rotary evaporator. The residue (19.5 g of a colourless resin) was heated at 200° C. for 1 h. The crude product obtained was reacted further without further purification.

LCMS (method 2): Rₜ=1.21 min; m/z [ES−]=247 (M−H)⁻

¹H-NMR (300 MHz, DMSO-d₆): δ=1.12 (s, 3H), 3.06 (s, 2H), 7.21-7.27 (m, 4H).

### Example 16A

### 2-Methyl-6-(trifluoromethoxy)indan-1-one

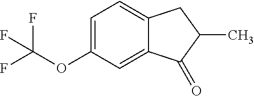 [SMILES: CC1Cc2ccc(OC(F)(F)F)cc2C1=O]

17.2 g (69.3 mmol) of crude 2-methyl-3-[4-(trifluoromethoxy)phenyl]propanoic acid (Example 15A) were dissolved in 100 ml of dichloromethane, and 12.1 ml (16.6 g, 166 mmol) of thionyl chloride and 0.16 ml of DMF were added dropwise at RT. The mixture was then heated under reflux for about 30 min until the evolution of gas had ceased. The solution was allowed to cool and the solvents were removed on a rotary evaporator. The residue (yellow solid) was taken up in 35 ml of dichloromethane and, at RT, added dropwise to a suspension of 10.2 g (76.2 mmol) of anhydrous aluminium chloride in 200 ml of dichloromethane. The dark-red solution was stirred for 30 min and then added to water and the phases were separated. The aqueous phase was extracted 3× with dichloromethane, washed with water, sat. sodium bicarbonate solution and sat. sodium chloride solution and dried with sodium sulphate. The solvents were removed and the residue (10.0 g) was purified by flash chromatography (SiO₂, hexane/dioxane). This gave 5.84 mg (14% of theory) of the product as a yellow oil.

LCMS (method 2): Rₜ=1.27 min; m/z=231; 272 (M+H)⁺/(M+ACN+H)⁺

### Example 17A

### 3-(4-Chlorophenyl)-2-methyl-5-(trifluoromethoxy)-1H-indene

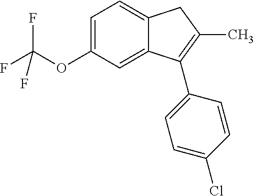 [SMILES: CC1=C(c2ccc(Cl)cc2)c2cc(OC(F)(F)F)ccc2C1]

Under argon, 38.1 ml of 4-chlorophenylmagnesium bromide (1M in diethyl ether, 38.1 mmol) were initially charged in 80 ml of THF, and 5.84 g (25.4 mmol) of 2-methyl-6-trifluoromethoxyindan-1-one (Example 16A), dissolved in 20 ml of THF, were added dropwise at RT. The mixture was stirred at RT for 1 h and then added to sat. ammonium chloride solution and extracted 3× with ethyl acetate, the combined organic phases were washed with sat. sodium chloride solution and dried with sodium sulphate and the solvents were removed on a rotary evaporator.

The residue was taken up in 375 ml of dichloromethane, 55 mg of 4-toluenesulphonic acid monohydrate were added and the mixture was stirred at RT for 16 h. The reaction mixture was added to sat. sodium hydrogencarbonate solution and extracted 3× with dichloromethane, the combined organic phases were washed with sat. sodium chloride solution and dried with sodium sulphate and the solvent was removed on a rotary evaporator. The residue was purified by flash chromatography (SiO₂, hexane/ethyl acetate). This gave 2.42 mg (21% of theory) of the product as a colourless resin.

LCMS (method 1): Rₜ=1.76 min; m/z=325 (M+H)⁺

¹H-NMR (400 MHz, DMSO-d₆): δ=2.09 (s, 3H), 3.53 (s, 2H), 6.93 (s, br, 1H), 7.07-7.12 (m, 1H), 7.38 (d, 2H), 7.50-7.56 (m, 1H), 7.54 (d, 2H).

### Example 18A

### tert-Butyl [3-(4-chlorobenzoyl)-4-(2-oxopropyl)phenyl]carbamate

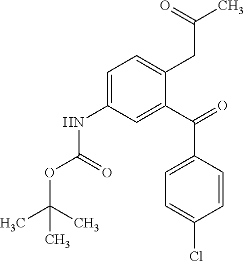 [SMILES: CC(=O)Cc1ccc(NC(=O)OC(C)(C)C)cc1C(=O)c1ccc(Cl)cc1]

22.0 g (61.8 mmol) of tert-butyl [3-(4-chlorophenyl)-2-methyl-1H-inden-5-yl]carbamate (Example 12A) were initially charged in 120 ml of hexane and 120 ml of acetonitrile, and 280 mg (1.24 mmol) of ruthenium(III) chloride hydrate (CAS [14898-67-0]) were added. The mixture was stirred at 0° C. for 10 min, and 26.4 g (124 mmol) of sodium periodate were then added a little at a time. The brown suspension was stirred for a further 90 min. The mixture was filtered through silica gel, the filter cake was washed with ethyl acetate, the filtrate was washed with sat. sodium chloride solution and dried with sodium sulphate and the solvents were removed on a rotary evaporator. The residue was purified by flash chromatography (SiO₂, hexane/ethyl acetate 0-20-50%). This gave 5.30 g (20% of theory) of the product as a yellow foam.

LCMS (method 2): Rₜ=1.39 min; m/z=388 (M+H)⁺

¹H-NMR (300 MHz, DMSO-d₆): δ=1.39 (s, 9H), 2.01 (s, 3H), 3.83 (s, 2H), 7.20 (d, 1H), 7.44 (d, 1H), 7.51-7.57 (m, 1H), 7.59 (d, 2H), 7.69 (d, 2H), 9.42 (s, 1H).

Analogously to Example 18A, the following compound was prepared from the corresponding 2-methyl-1H-indene:

| No | Structure | Name | Analytical data |
|---|---|---|---|
| 19A | 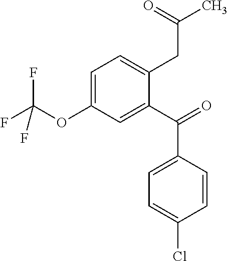 [SMILES: CC(=O)Cc1ccc(OC(F)(F)F)cc1C(=O)c1ccc(Cl)cc1] | 1-[2-(4-chlorobenzoyl)- 4- (trifluoromethoxy) phenyl]propan-2-one | ¹H-NMR (400 MHz, DMSO-d₆): δ = 2.05 (s, 3H), 3.98 (s, 2H), 7.31-7.33 (m, 1H), 7.46 (d, 1H), 7.50-7.56 (m, 1H), 7.60 (d, 2H), 7.68 (d, 2H), LCMS (method 1): Rₜ = 1. 45 min; m/z = 357 (M + H)⁺ |

### Example 20A

### 1-(4-Bromophenyl)-7,8-dimethoxy-4-methyl-5H-2,3-benzodiazepine

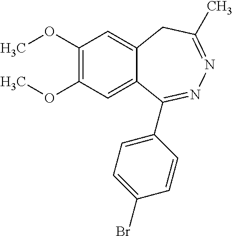

730 mg (1.94 mmol) of 1-[2-(4-bromobenzoyl)-4,5-dimethoxyphenyl]propan-2-one (Example 5A) and 513 mg (10.3 mmol) of hydrazine hydrate were stirred in 7 ml of ethanol at a bath temperature of 100° C. for 1 h. After cooling, the mixture was saturated with hydrogen chloride gas (introduced for about 5 min). The reaction solution was added to water, made alkaline with 1M aqueous sodium hydroxide solution and extracted with dichloromethane. The combined organic phases were dried with sodium sulphate and the solvent was removed on a rotary evaporator. The residue (1 g of a yellow solid) was purified by flash chromatography (SiO₂, dichloromethane/methanol 0-3%). This gave 390 mg (50% of theory) of the product as a yellow solid.

LCMS (method 2): Rₜ=1.20 min; m/z=373; 375 (Br isotope pattern, M+H)⁺

¹H-NMR (300 MHz, DMSO-d₆): δ=2.02 (s, 3H), 2.71 (d, 1H), 3.42 (d, 1H), 3.59 (s, 3H), 3.83 (s, 3H), 6.70 (s, 1H), 7.06 (s, 1H), 7.50 (d, 2H), 7.61 (d, 2H).

Analogously to Example 20A, the following compounds were prepared from the corresponding diketones:

| No | Structure | Name | Analytical data |
|---|---|---|---|
| 21A | 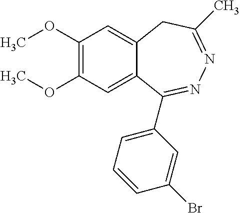 | 1-(3-bromophenyl)- 7,8-dimethoxy-4- methyl-5H-2,3- benzodiazepine | ¹H-NMR (300 MHz, CDCl₃): δ = 2.15 (s, 3H), 2.98 (d, 1H), 3.27 (d, 1H), 3.75 (s, 3H), 3.97 (s, 3H), 6.73 (s, 1H), 6.75 (s, 1H), 7.27 (dd, 1H), 7.55 (dbr, 1H), 7.61 (dbr, 1H), 7.86 (m, 1H). LCMS (method 3): Rₜ = 1.15 min; m/z = 373; 375 (M + H, Br isotope pattern)⁺ |
| 22A | 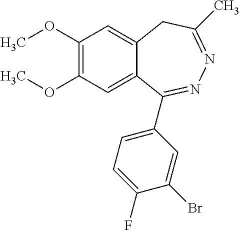 | 1-(3-bromo-4- fluorophenyl)-7,8- dimethoxy-4-methyl- 5H-2,3-benzodiazepine | ¹H-NMR (400 MHz, CDCl₃): δ = 2.16 (s, 3H), 2.99 (d, 1H), 3.29 (d, 1H), 3.77 (s, 3H), 3.98 (s, 3H), 6.74 (s, 2H), 7.16 (dd, 1H), 7.62 (ddd, 1H), 7.94 (dd, 1H). LCMS (method 3): Rₜ = 1.21 min; m/z = 381; 383 (Br isotope pattern, M + H)⁺ |
| 23A | 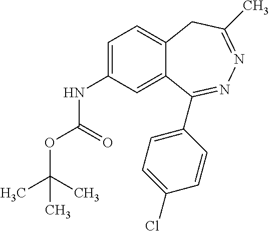 [SMILES: CC1=NN=C(c2ccc(Cl)cc2)c2cc(NC(=O)OC(C)(C)C)ccc2C1] | tert-butyl [1-(4- chlorophenyl)-4- methyl-5H-2,3- benzodiazepin-8- yl]carbamate | ¹H-NMR (400 MHz, CDCl₃): δ = 1.48 (s, 9H), 2.13 (s, 3H), 3.03 (d, 1H), 3.30 (d, 1H), 6.48 (s, br, 1H), 7.11 (d, 1H), 7.22 (d, 1H), 7.37 (d, 2H), 7.62 (d, 2H), 7.73 (d, 1H). LCMS (method 1): Rₜ = 1.37 min; m/z = 384 (M + H)⁺ |
| 24A | 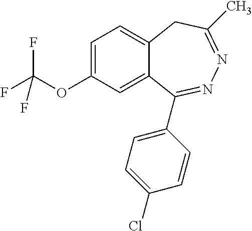 [SMILES: CC1=NN=C(c2ccc(Cl)cc2)c2cc(OC(F)(F)F)ccc2C1] | 1-(4-chlorophenyl)-4- methyl-8- (trifluoromethoxy)-5H- 2,3-benzodiazepine | ¹H-NMR (400 MHz, DMSO-d₆): δ = 2.05 (s, 3H), 2.89 (d, 1H), 3.61 (d, 1H), 7.20 (s, br, 1H), 7.49-7.54 (m, 4H), 7.59-7.66 (m, 2H). LCMS (method 1): Rₜ = 1.44 min; m/z = 353 (M + H)⁺ |

### Example 25A

### (±)-1-(4-Bromophenyl)-7,8-dimethoxy-4-methyl-4,5-dihydro-3H-2,3-benzodiazepine

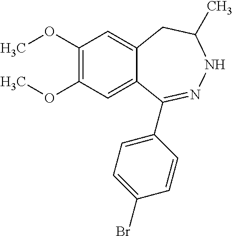

At RT, 1.99 g (5.33 mmol) of 1-(4-bromophenyl)-7,8-dimethoxy-4-methyl-5H-2,3-benzodiazepine (obtained as described in Example 20A) were initially charged in 200 ml of methanol, 3.0 ml of 2M hydrochloric acid were added and 1.68 g (26.6 mmol) of sodium cyanoborohydride were introduced. The mixture was stirred at RT for 1 h and then made alkaline with 2M aqueous sodium hydroxide solution (pH about 8). Most of the methanol was removed on a rotary evaporator, and the residue was partitioned between water and dichloromethane. The phases were separated and the aqueous phase was extracted with dichloromethane. The combined organic phases were washed with sat. sodium chloride solution and dried with sodium sulphate and the solvent was removed on a rotary evaporator. The residue was purified by flash chromatography (SiO₂, hexane/ethyl acetate). This gave 1.56 mg (78% of theory) of the product as a yellow resin which crystallized.

LCMS (method 2): Rₜ=0.96 min; m/z=375; 377 (Br isotope pattern, M+H)⁺

¹H-NMR (400 MHz, DMSO-d₆): δ=1.09 (d, 3H), 2.58 (dd, 1H), 2.83 (dd, 1H), 3.27 (s, 3H), 3.51 (s, 3H), 3.77-3.82 (m, 1H), 6.47 (s, 1H), 6.85 (s, 1H), 7.01 (d, 1H), 7.33 (d, 2H), 7.47 (d, 2H).

Analogously to Example 25A, the following compounds were prepared from the corresponding 5H-2,3-benzodiazepines:

| No | Structure | Name | Analytical data |
|---|---|---|---|
| 26A | 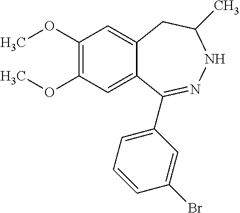 | (±)-1-(3-bromophenyl)- 7,8-dimethoxy-4- methyl-4,5-dihydro- 3H-2,3-benzodiazepine | ¹H-NMR (400 MHz, CDCl₃): δ = 1.28 (d, 3H), 2.62 (dd, 1H), 2.89 (dd, 1H), 3.71 (s, 3H), 3.94 (s, 3H), 4.11 (m, 1H), 6.59 (s, 1H), 6.76 (s, 1H), 7.22 (dd, 1H), 7.45 (dbr, 1H), 7.48 (dbr, 1H), 7.75 (m, 1H). LCMS (method 3): Rₜ = 0.99 min; m/z = 375; 377 (M + H, Br isotope pattern)⁺ |
| 27A | 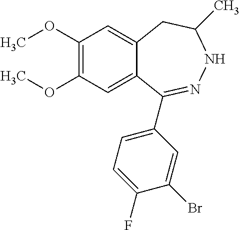 | (±)-1-(3-bromo-4- fluorophenyl)-7,8- dimethoxy-4-methyl- 4,5-dihydro-3H-2,3- benzodiazepine | ¹H-NMR (400 MHz, CDCl₃): δ = 1.29 (d, 3H), 2.61 (dd, 1H), 2.89 (dd, 1H), 3.72 (s, 3H), 3.95 (s, 3H), 4.12 (m, 1H), 6.57 (s, 1H), 6.77 (s, 1H), 7.10 (dd, 1H), 7.45 (ddd, 1H), 7.81 (dd, 1H). LCMS (method 3): Rₜ = 1.03 min; m/z = 393; 395 (Br isotope pattern, M + H)⁺ |
| 28A | 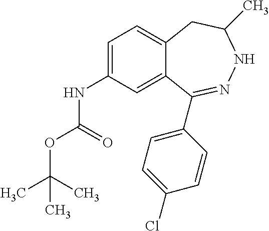 [SMILES: CC1Cc2ccc(NC(=O)OC(C)(C)C)cc2C(c2ccc(Cl)cc2)=NN1] | (±)-tert-butyl [1-(4- chlorophenyl)-4- methyl-4,5-dihydro- 3H-2,3-benzodiazepin- 8-yl]carbamate | ¹H-NMR (400 MHz, DMSO-d₆): δ = 1.60 (d, 3H), 1.37 (s, 9H), 2.58 (dd, 1H), 2.82 (dd, 1H), 3.75-3.81 (m, 1H), 7.07 (d, 1H), 7.09 (d, 1H), 7.14 (d, 1H), 7.33-7.38 (m, 4H), 9.16 (s, br, 1H). LCMS (method 2): Rₜ = 1.23 min; m/z = 386 (M + H)⁺ |
| 29A | 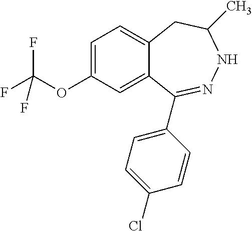 [SMILES: CC1Cc2ccc(OC(F)(F)F)cc2C(c2ccc(Cl)cc2)=NN1] | (±)-1-(4-chlorophenyl)- 4-methyl-8- (trifluoromethoxy)-4,5- dihydro-3H-2,3- benzodiazepine | ¹H-NMR (400 MHz, DMSO-d₆): δ = 1.10 (d, 3H), 2.75 (dd, 1H), 2.99 (dd, 1H), 3.76-3.83 (m, 1H), 6.84 (s, br, 1H), 7.21-7.24 (m, 1H), 7.32-7.38 (m,5H), 7.64 (s, br, 1H). LCMS (method 2): Rₜ = 1.50 min; m/z = 355 (M + H)⁺ |

### Example 30A

### (±)-1-(4-Bromophenyl)-7,8-dimethoxy-N,4-dimethyl-4,5-dihydro-3H-2,3-benzodiazepine-3-carboxamide

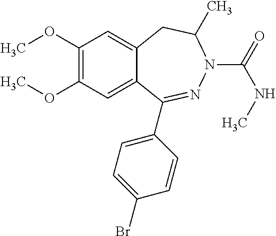 [SMILES: CNC(=O)N1N=C(c2ccc(Br)cc2)c2cc(OC)c(OC)cc2CC1C]

At RT, 1.56 g (4.16 mmol) of (±)-1-(4-bromophenyl)-7,8-dimethoxy-4-methyl-4,5-dihydro-3H-2,3-benzodiazepine (Example 25A) were dissolved in 50 ml of THF, 1.68 g (8.31 mmol) of 4-nitrophenyl chloroformate (CAS [7693-46-1]) were added dropwise and the mixture was stirred at RT for 1 h. During this time, the clear yellow solution slowly became turbid. 20.8 ml (41.6 mmol) of a 2M solution of methylamine in THF were added dropwise and the mixture was stirred at 60° C. for 5 h. The mixture was allowed to cool to RT, concentrated on a rotary evaporator and partitioned between water and ethyl acetate and the phases were separated. The aqueous phase was extracted with ethyl acetate. The combined organic phases were washed with sat. sodium chloride solution and dried with sodium sulphate and the solvent was removed on a rotary evaporator. If the reaction of the intermediate 4-nitrophenyl carbamate with methylamine was incomplete (monitored by UPLC/MS), the reaction of methylamine with the crude product/intermediate mixture can be repeated analogously to achieve complete conversion. The crude product was purified by flash chromatography (SiO₂, hexane/ethyl acetate). This gave 1.90 g (100% of theory) of the desired product as a yellow foam.

LCMS (method 2): Rₜ=1.33 min; m/z=432; 434 (Br isotope pattern, M+H)⁺

¹H-NMR (400 MHz, DMSO-d₆): δ=0.92 (d, 3H), 2.64 (d, 3H), 2.67 (dd, 1H), 2.91 (dd, 1H), 3.53 (s, 3H), 3.80 (s, 3H), 5.03-5.11 (m, 1H), 6.47 (s, 1H), 6.60 (q, 1H), 6.98 (s, 1H), 7.56 (s, 4H).

### Enantiomer Separation

19.9 g of the compound prepared by the process described under 30A were separated into the enantiomers by chiral preparative HPLC under the following conditions:

system: SFC Prep 400; column: Chiralpak AZ-H 5 μm 250×50 mm; mobile phase: CO₂/isopropanol 75:25 (v/v); flow rate: 300 ml/min; temperature: 38° C.; pressure 80 bar; solution: 5 g/100 ml of methanol/acetonitrile 50:50 (v/v); detection: UV 220 nm.

### Example 30.1A

(4R)-1-(4-Bromophenyl)-7,8-dimethoxy-N,4-dimethyl-4,5-dihydro-3H-2,3-benzodiazepine-3-carboxamide

9.29 g, light-yellow solid, HPLC (Method F): Rₜ=3.29 min, purity>99%

optical rotation: [α]_{D}²⁰=−89.3° (c=1.00; methanol)

### Example 30.2A

### (4S)-1-(4-Bromophenyl)-7,8-dimethoxy-N,4-dimethyl-4,5-dihydro-3H-2,3-benzodiazepine-3-carboxamide

9.9 g, light-yellow solid, HPLC (Method F): Rₜ=4.55 min, purity 96%

optical rotation: [α]_{D}²⁰=+81.3° (c=1.00; methanol)

Analogously to Example 30A, the following compounds were prepared from the corresponding 4,5-dihydro-3H-2,3-benzodiazepines:

| No | Structure | Name | Analytical data |
|---|---|---|---|
| 31A | 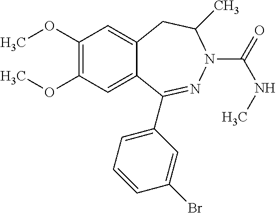 [SMILES: CNC(=O)N1N=C(c2cccc(Br)c2)c2cc(OC)c(OC)cc2CC1C] | (±)-1-(3-bromophenyl)- 7,8-dimethoxy-N,4- dimethyl-4,5-dihydro- 3H-2,3- benzodiazepine-3- carboxamide | ¹H-NMR (500 MHz, CDCl₃): δ = 0.95 (d, 3H), 2.86 (dd, 1H), 2.90 (d, 3H), 3.12 (dd, 1H), 3.66 (s, 3H), 3.93 (s, 3H), 5.48 (m, 1H), 6.50 (m, 1H), 6.54 (s, 1H), 6.71 (s, 1H), 7.26 (dd, 1H), 7.39 (dbr, 1H), 7.52 (dbr, 1H), 7.64 (m, 1H). LCMS (method 3): Rₜ = 1.27 min; m/z = 432; 434 (M + H, Br isotope pattern)⁺ |
| 32A | 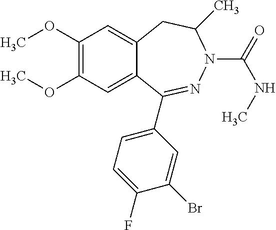 [SMILES: CNC(=O)N1N=C(c2ccc(F)c(Br)c2)c2cc(OC)c(OC)cc2CC1C] | (±)-1-(3-bromo-4- fluorophenyl)-7,8- dimethoxy-N,4- dimethyl-4,5-dihydro- 3H-2,3- benzodiazepine-3- carboxamide | ¹H-NMR (400 MHz, CDCl₃): δ = 0.95 (d, 3H), 2.86 (dd, 1H), 2.90 (d, 3H), 3.10 (dd, 1H), 3.67 (s, 3H), 3.93 (s, 3H), 5.48 (m, 1H), 6.44 (m, 1H), 6.52 (s, 1H), 6.71 (s, 1H), 7.14 (dd, 1H), 7.39 (ddd, 1H), 7.69 (dd, 1H). LCMS (method 3): Rₜ = 1.31 min; m/z = 450; 452 (Br isotope pattern, M + H)⁺ |
| 33A | 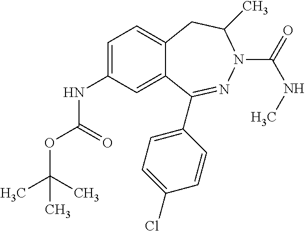 [SMILES: CNC(=O)N1N=C(c2ccc(Cl)cc2)c2cc(NC(=O)OC(C)(C)C)ccc2CC1C] | (±)-tert-butyl [1-(4- chlorophenyl)-4- methyl-3- (methylcarbamoyl)- 4,5-dihydro-3H-2,3- benzodiazepin-8- yl]carbamate | ¹H-NMR (400 MHz, DMSO-d₆): δ = 0.89 (d, 3H), 1.37 (s, 9H), 2.60-2.66 (m, 1H), 2.63 (d, 3H), 2.90 (dd, 1H), 4.97-5.05 (m, 1H), 6.61 (q, 1H), 7.14 (d, 1H), 7.21 (d, 1H), 7.43-7.47 (m, 1H), 7.45 (d, 2H), 7.62 (d, 2H), 9.30 (s, br, 1H). LCMS (method 2): Rₜ = 1.45 min; m/z = 443 (M+H)⁺ |
| 34A | 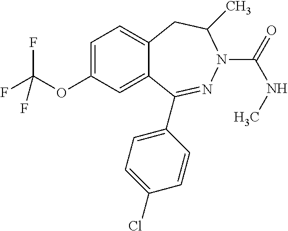 [SMILES: CNC(=O)N1N=C(c2ccc(Cl)cc2)c2cc(OC(F)(F)F)ccc2CC1C] | (±)-1-(4-chlorophenyl)- N,4-dimethyl-8- (trifluoromethoxy)-4,5- dihydro-3H-2,3- benzodiazepine-3- carboxamide | ¹H-NMR (300 MHz, CDCl₃): δ = 0.91 (d, 3H), 2.90 (d, 3H), 2.96 (dd, 1H), 3.14 (dd, 1H), 5.46-5.55 (m, 1H), 6.47-6.52 (m, 1H), 6.94 (s, br, 1H), 7.17-7.29 (m, 2H), 7.39 (s, 4H). LCMS (method 2): Rₜ = 1.53 min; m/z = 412; 414 (Cl isotope pattern, M + H)⁺ |

### Example 35A

### (±)-8-Amino-1-(4-chlorophenyl)-N,4-dimethyl-4,5-dihydro-3H-2,3-benzodiazepine-3-carboxamide

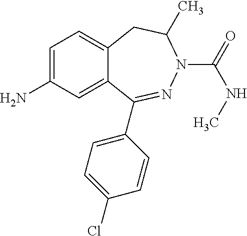 [SMILES: CNC(=O)N1N=C(c2ccc(Cl)cc2)c2cc(N)ccc2CC1C]

4.50 g (10.2 mmol) of (±)-tert-butyl [1-(4-chlorophenyl)-4-methyl-3-(methylcarbamoyl)-4,5-dihydro-3H-2,3-benzodiazepin-8-yl]carbamate (Example 33A) were initially charged in 100 ml of dichloromethane, 15 ml (20.3 mmol) of trifluoroacetic acid were added at 0° C. and the mixture was then stirred at RT for another 4 h. The mixture was carefully added to 20% strength potassium carbonate solution and extracted with dichloromethane. The combined organic phases were dried with sodium sulphate and the solvents were removed on a rotary evaporator. This gave 3.40 g (97% of theory) of the desired product as a brownish solid.

LCMS (method 2): Rₜ=1.12 min; m/z=343 (M+H)⁺

¹H-NMR (300 MHz, DMSO-d₆): δ=0.88 (d, 3H), 2.52 (dd, 1H), 2.63 (d, 3H), 2.80 (dd, 1H), 4.89-5.05 (m, 1H), 5.01 (s, br, 2H), 6.19 (d, 1H), 6.52-6.59 (m, 2H), 6.96 (d, 1H), 7.44 (d, 1H), 7.61 (d, 2H).

### Example 36A

### (±)-1-(4-Chlorophenyl)-8-(3,5-dimethyl-1H-pyrazol-1-yl)-N,4-dimethyl-4,5-dihydro-3H-2,3-benzodiazepine-3-carboxamide

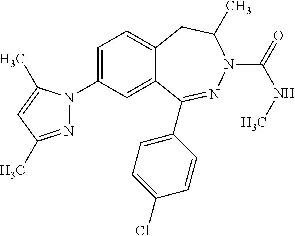 [SMILES: CNC(=O)N1N=C(c2ccc(Cl)cc2)c2cc(-n3nc(C)cc3C)ccc2CC1C]

Under argon, 1.0 g (2.9 mmol) of (±)-8-amino-1-(4-chlorophenyl)-N,4-dimethyl-4,5-dihydro-3H-2,3-benzodiazepine-3-carboxamide (Example 35A) was dissolved in 40 ml of concentrated hydrochloric acid, and the mixture was cooled to 0° C. Over a period of 25 min, a solution of 240 mg (3.50 mmol) of sodium nitrite in 10 ml of water was metered in, and the mixture was stirred at this temperature for 30 min. A solution of 1.65 g (7.29 mmol) of tin(II) chloride in 8 ml of concentrated hydrochloric acid was then slowly added dropwise over 30 min. The ice bath was removed and the mixture was stirred at RT for another 45 min. 60 μl (5.8 mmol) of 2,4-pentanedione were then added, and the mixture was stirred for another 30 min. Finally, 20 ml of acetonitrile were added and the mixture was stirred at RT for another 1 h. The mixture was added to ice-water, adjusted to pH 10 with aqueous sodium hydroxide solution and extracted three times with dichloromethane. The solvent was removed on a rotary evaporator. This gave 1.16 g (88% of theory) of the desired product which was converted further without further purification.

LCMS (method 1): Rₜ=1.38 min; m/z=422 (M+H)⁺

¹H-NMR (300 MHz, DMSO-d₆): δ=0.91 (d, 3H), 2.07 (s, 3H), 2.17 (s, 3H), 2.65 (d, 3H), 2.83 (dd, 1H), 3.05 (dd, 1H), 5.10-5.18 (m, 1H), 5.98 (s, 1H), 6.71 (q, 1H), 7.02 (d, 1H), 7.45 (d, 2H), 7.43-7.53 (m, 2H), 7.64 (d, 2H).

### Preparation of the Compounds According to the Invention

### Example 1

### [1S-(1R*,4S*)]-7,8-Dimethoxy-N,4-dimethyl-1-[4-(3-oxo-2-azabicyclo[2.2.1]hept-2-yl)phenyl]-4,5-dihydro-3H-2,3-benzodiazepine-3-carboxamide

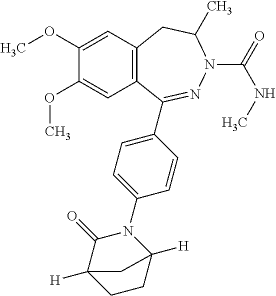 [SMILES: CNC(=O)N1N=C(c2ccc(N3C(=O)C4CCC3C4)cc2)c2cc(OC)c(OC)cc2CC1C]

Under argon, 100 mg (0.231 mmol) of (±)-1-(4-bromophenyl)-7,8-dimethoxy-N,4-dimethyl-4,5-dihydro-3H-2,3-benzodiazepine-3-carboxamide (Example 30A), 28 mg (0.254 mmol) of (±)-2-azabicyclo[2.2.1]heptan-3-one (CAS [24647-29-8]), 98 mg (0.46 mmol) of potassium phosphate and 88 mg (0.46 mmol) of copper(I) iodide were initially charged in 4 ml of degassed dioxane. 82 mg (0.93 mmol) of N,N-dimethylethylenediamine were then added under argon and the mixture was degassed again and heated at 130° C. for 3 hours. After cooling, ethyl acetate and saturated aqueous ammonium chloride solution were added to the mixture. The aqueous phase was extracted three more times with ethyl acetate, and then the combined organic phases were dried with sodium sulphate. The solvent was removed on a rotary evaporator and the residue was purified by preparative RP-HPLC. This gave 56 g (52% of theory) of the desired product (stereoisomer mixture) as a solid.

LCMS (method 2): Rₜ=1.0 min; m/z=463 (M+H)⁺

¹H-NMR (500 MHz, DMSO-d6): δ=1.01 (dd, 3H), 1.54-1.61 (m, 2H), 1.69-1.77 (m, 1H), 1.92-2.04 (m, 3H), 2.57-2.65 (m, 1H), 2.67 (dd, 3H), 2.84 (br. s., 1H), 2.90 (dd, 1H), 3.59 (s, 3H), 3.84 (s, 3H), 4.67 (d, 1H), 4.98-5.07 (m, 1H), 6.44-6.51 (m, 1H), 6.53 (s, 1H), 7.01 (s, 1H), 7.58-7.62 (m, 2H), 7.64-7.68 (m, 2H).

Analogously to Example 1, Example 30.2A and the appropriate commercially available amide (CAS[134003-03-5]) gave the following exemplary compound:

| | | | |
|---|---|---|---|
| 2 | 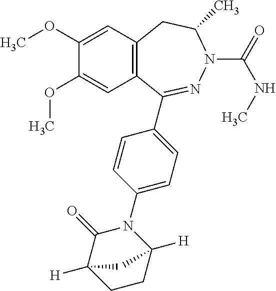 [SMILES: CNC(=O)N1N=C(c2ccc(N3C(=O)[C@@H]4CC[C@H]3C4)cc2)c2cc(OC)c(OC)cc2C[C@@H]1C] | {1S-[1R*,2(S*),4S*]}- 7,8-dimethoxy-N,4- dimethyl-1-[4-(-3-oxo-2- azabicyclo[2.2.1]hept-2- yl)phenyl]-4,5-dihydro- 3H-2,3-benzodiazepine- 3-carboxamide | LCMS (method 2): Rₜ = 1.11 min; m/z = 463 (M + H)⁺ [α]_{D}²⁰ = 205.5° (c = 1.00; methanol) |

### Example 3.1

### (4S)-1-[4-(1,1-Dioxido-1-thia-6-azaspiro[3.3]hept-6-yl)phenyl]-7,8-dimethoxy-N,4-dimethyl-4,5-dihydro-3H-2,3-benzodiazepine-3-carboxamide

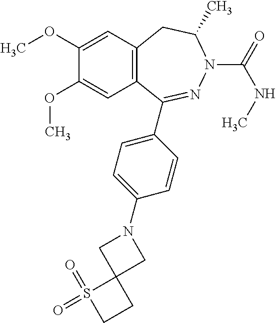 [SMILES: CNC(=O)N1N=C(c2ccc(N3CC4(CCS4(=O)=O)C3)cc2)c2cc(OC)c(OC)cc2C[C@@H]1C]

Under argon, 1.00 g (2.31 mmol) of (4S)-1-(4-bromophenyl)-7,8-dimethoxy-N,4-dimethyl-4,5-dihydro-3H-2,3-benzodiazepine-3-carboxamide (Example 30.2A) was initially charged in 35 ml of degassed toluene. 2.41 g (9.25 mmol) of 1,1-dioxo-1-thia-6-azaspiro[3.3]heptane trifluoroacetate (free base CAS[1352546-75-8], 445 mg (4.62 mmol) of sodium tert-butoxide and 91 mg (0.12 mmol) of chloro-(2-dicyclohexylphosphino-2,4,6-triisopropyl-1,1-biphenyl)[2-(2-amino-1,1-biphenyl)]palladium(II) (CAS [1310584-14-5]) were added. The mixture was degassed again and saturated with argon and then stirred at 80° C. for 7 hours. After cooling, the mixture was added to sat. sodium bicarbonate solution and extracted with ethyl acetate. The combined organic phases were washed with sat. sodium chloride solution and dried with sodium sulphate. The solvents were removed on a rotary evaporator and the residue (1.6 g of an orange foam) was purified by flash chromatography (SiO₂, dichloromethane/methanol 0-3-5%). This gave 570 mg (49% of theory) of the desired product as a yellow solid.

LCMS (method 1): Rₜ=1.03 min; m/z=499 (M+H)⁺

¹H-NMR (300 MHz, DMSO-d6): δ=1.02 (d, 3H), 2.33-2.41 (m, 2H), 2.40-2.50 (m, 1H), 2.59 (d, 3H), 2.81 (dd, 1H), 3.55 (s, 3H), 3.79 (s, 3H), 4.04-4.14 (m, 4H), 4.33-4.40 (m, 2H), 4.81-4.92 (m, 1H), 6.26 (q, 1H), 6.47 (s, 1H), 6.53 (d, 2H), 6.99 (s, 1H), 7.56 (d, 2H).

Specific optical rotation: [α]_{D}²⁰=355° (c=1.00; methanol)

### Example 3.2

### (4R)-1-[4-(1,1-Dioxido-1-thia-6-azaspiro[3.3]hept-6-yl)phenyl]-7,8-dimethoxy-N,4-dimethyl-4,5-dihydro-3H-2,3-benzodiazepine-3-carboxamide

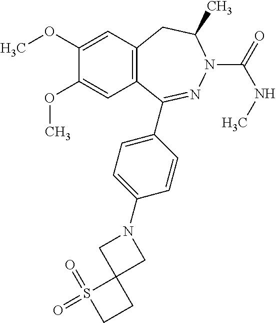 [SMILES: CNC(=O)N1N=C(c2ccc(N3CC4(CCS4(=O)=O)C3)cc2)c2cc(OC)c(OC)cc2C[C@H]1C]

Analogously to the preparation of Example 3.1, using (4R)-1-(4-bromophenyl)-7,8-dimethoxy-N,4-dimethyl-4,5-dihydro-3H-2,3-benzodiazepine-3-carboxamide (Example 30.1A), Example 3.2 was prepared as a solid.

LCMS (method 1): Rₜ=1.04 min; m/z=499 (M+H)⁺

Specific optical rotation: [α]_{D}²⁰=−326.7° (c=1.00; methanol)

Analogously to Example 3.1, Example 30A and the appropriate commercially available amines:

| CAS number | Name |
|---|---|
| 1352546-75-8 | 1-thia-6-azaspiro[3.3]heptane-1,1-dioxide trifluoroacetate |
| (free base) | |
| 54745-74-3 | 8-oxa-3-azabicyclo[3.2.1]octane hydrochloride (1:1) |
| 1045709-32-7 | ethanedioic acid - 2-oxa-6-azaspiro[3.3]heptane (1:2) |
| 25602-68-0 | 8-azabicyclo[3.2.1]octan-3-one hydrochloride (1:1) |
| 1041026-71-4 | ethanedioic acid - tert-butyl |
| | 2,6-diazaspiro[3.3]heptane-2-carboxylate (1:2) |
| 176-64-7 | 8-azaspiro[4.5]decane |
| 1394840-24-4 | ethanedioic acid - 2-benzyl-2,6- |
| | diazaspiro[3.3]heptane (1:1) |
| 11046153-20-1 | 2-oxa-6-azaspiro[3.5]nonane |
| 241820-91-7 | 2-oxa-7-azaspiro[3.5]nonane |

optionally followed by enantiomer separation using the preparative HPLC method indicated in each case, gave the following exemplary compounds:

| No | Structure | Name | Analytical data |
|---|---|---|---|
| 3 | 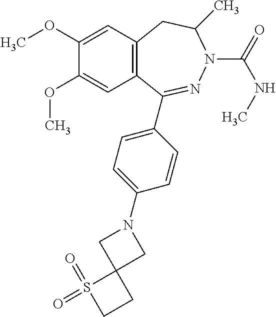 [SMILES: CNC(=O)N1N=C(c2ccc(N3CC4(CCS4(=O)=O)C3)cc2)c2cc(OC)c(OC)cc2CC1C] | 1-[4-(1,1-dioxido-1- thia-6-azaspiro[3.3]hept- 6-yl)phenyl]-7,8- dimethoxy-N,4-dimethyl- 4,5-dihydro-3H-2,3- benzodiazepine-3- carboxamide | ¹H-NMR (300 MHz, DMSO-d₆): δ = 1.02 (d, 3H), 2.33-2.41 (m, 2H), 2.40-2.50 (m, 1H), 2.60 (d, 3H), 2.81 (dd, 1H), 3.56 (s, 3H), 3.80 (s, 3H), 4.04-4.14 (m, 4H), 4.33-4.40 (m, 2H), 4.81-4.92 (m, 1H), 6.26 (q, 1H), 6.48 (s, 1H), 6.53 (d, 2H), 6.99 (s, 1H), 7.56 (d, 2H). LCMS (method 2): Rₜ = 1.03 min; m/z = 499 (M + H)⁺ |
| 4 | 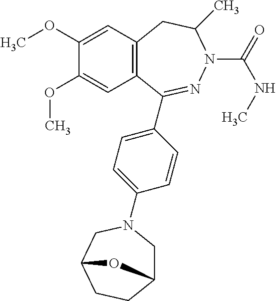 [SMILES: CNC(=O)N1N=C(c2ccc(N3C[C@H]4CC[C@@H](C3)O4)cc2)c2cc(OC)c(OC)cc2CC1C] | (±)-7,8-dimethoxy-N,4- dimethyl-1-[4-(8-oxa-3- azabicyclo[3.2.1]oct-3- yl)phenyl]-4,5-dihydro- 3H-2,3-benzodiazepine- 3-carboxamide | ¹H-NMR (300 MHz, DMSO-d₆): δ = 1.01 (d, 3H), 1.75-1.83 (m, 4H), 2.40 (dd, 1H), 2.59 (d, 3H), 2.80 (dd, 1H), 2.81-2.87 (m, 2H), 3.44-3.48 (m, 2H), 3.58 (s, 3H), 3.80 (s, 3H), 4.36-4.47 (m, 2H), 4.79-4.91 (m, 1H), 6.25 (q, 1H), 6.51 (s, 1H), 6.82 (d, 2H), 6.99 (s, 1H), 7.55 (d, 2H). LCMS (method 2): Rₜ = 1.15 min; m/z = 465 (M + H)⁺ |
| 4.1 | 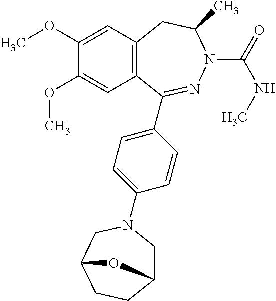 [SMILES: CNC(=O)N1N=C(c2ccc(N3C[C@H]4CC[C@@H](C3)O4)cc2)c2cc(OC)c(OC)cc2C[C@H]1C] | (4R)-7,8-dimethoxy-N,4- dimethyl-1-[4-(8-oxa-3- azabicyclo[3.2.1]oct-3- yl)phenyl]-4,5-dihydro- 3H-2,3-benzodiazepine- 3-carboxamide | prep. HPLC Method I analyt. HPLC (Method A): Rₜ = 5.1 min |
| 4.2 | 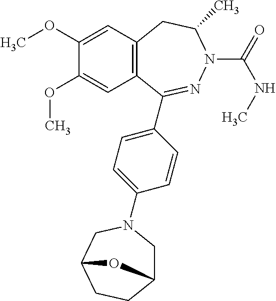 [SMILES: CNC(=O)N1N=C(c2ccc(N3C[C@H]4CC[C@@H](C3)O4)cc2)c2cc(OC)c(OC)cc2C[C@@H]1C] | (4S)-7,8-dimethoxy-N,4- dimethyl-1-[4-(8-oxa-3- azabicyclo[3.2.1]oct-3- yl)phenyl]-4,5-dihydro- 3H-2,3-benzodiazepine- 3-carboxamide | prep. HPLC Method I analyt. HPLC (Method A): Rₜ = 7.3 min |
| 5 | 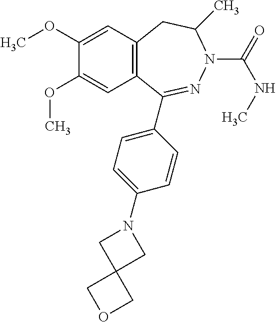 [SMILES: CNC(=O)N1N=C(c2ccc(N3CC4(COC4)C3)cc2)c2cc(OC)c(OC)cc2CC1C] | (±)-7,8-dimethoxy-N,4- dimethyl-1-[4-(2-oxa-6- azaspiro[3.3]hept-6- yl)phenyl]-4,5-dihydro- 3H-2,3-benzodiazepine- 3-carboxamide | ¹H-NMR (300 MHz, DMSO-d₆): δ = 1.02 (d, 3H), 2.36-2.50 (m, 1H), 2.59 (d, 3H), 2.79 (dd, 1H), 3.56 (s, 3H), 3.80 (s, 3H), 4.03 (s, 4H), 4.70 (s, 4H), 4.78-4.88 (m, 1H), 6.20 (q, 1H), 6.40 (d, 2H), 6.47 (s, 1H), 6.98 (s, 1H), 7.53 (d, 2H). LCMS (method 2): Rₜ = 1.03 min; m/z = 451 (M + H)⁺ |
| 5.1 | 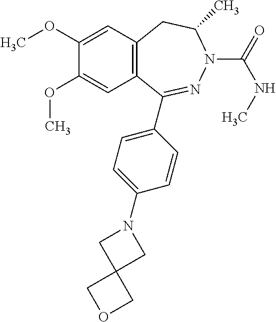 [SMILES: CNC(=O)N1N=C(c2ccc(N3CC4(COC4)C3)cc2)c2cc(OC)c(OC)cc2C[C@@H]1C] | (4S)-7,8-dimethoxy-N,4- dimethyl-1-[4-(2-oxa-6- azaspiro[3.3]hept-6- yl)phenyl]-4,5-dihydro- 3H-2,3-benzodiazepine- 3-carboxamide | prep. HPLC Method II analyt. HPLC (Method B): Rₜ = 2.63 min |
| 5.2 | 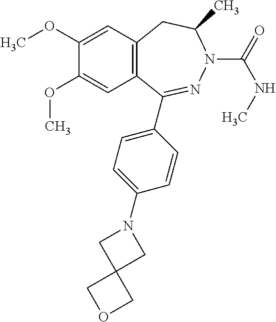 [SMILES: CNC(=O)N1N=C(c2ccc(N3CC4(COC4)C3)cc2)c2cc(OC)c(OC)cc2C[C@H]1C] | (4R)-7,8-dimethoxy-N,4- dimethyl-1-[4-(2-oxa-6- azaspiro[3.3]hept-6- yl)phenyl]-4,5-dihydro- 3H-2,3-benzodiazepine- 3-carboxamide | prep. HPLC Method II analyt. HPLC (Method B): Rₜ = 3.41 min |
| 6 | 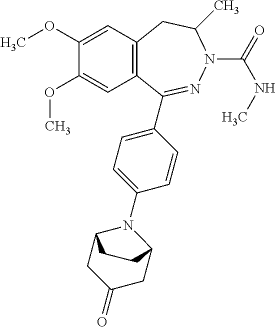 [SMILES: CNC(=O)N1N=C(c2ccc(N3[C@@H]4CC[C@H]3CC(=O)C4)cc2)c2cc(OC)c(OC)cc2CC1C] | (±)-7,8-dimethoxy-N,4- dimethyl-1-[4-(3-oxo-8- azabicyclo[3.2.1]oct-8- yl)phenyl]-4,5-dihydro- 3H-2,3-benzodiazepine- 3-carboxamide | ¹H-NMR (300 MHz, DMSO-d₆): δ = 1.03 (d, 3H), 1.05-1.09 (m, 2H), 1.62-1.70 (m, 2H), 2.01-2.10 (m, 2H), 2.15-2.25 (m, 2H), 2.39-2.50 (m, 1H), 2.60 (d, 3H), 2.80 (dd, 1H), 3.58 (s, 3H), 3.80 (s, 3H), 4.55-4.64 (m, 2H), 4.80-4.92 (m, 1H), 6.24 (q, 1H), 6.54 (s, 1H), 6.97 (d, 2H), 7.00 (s, 1H), 7.62 (d, 2H). LCMS (method 2): Rₜ = 1.12 min; m/z = 477 (M + H)⁺ |
| 6.1 | 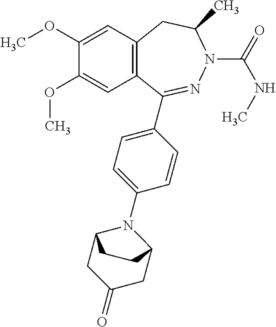 [SMILES: CNC(=O)N1N=C(c2ccc(N3[C@@H]4CC[C@H]3CC(=O)C4)cc2)c2cc(OC)c(OC)cc2C[C@H]1C] | (4R)-7,8-dimethoxy-N,4 dimethyl-1-[4-(3-oxo-8- azabicyclo[3.2.1]oct-8- yl)phenyl]-4,5-dihydro- 3H-2,3-benzodiazepine- 3-carboxamide | prep. HPLC Method III analyt. HPLC (Method E): Rₜ = 4.80 min |
| 6.2 | 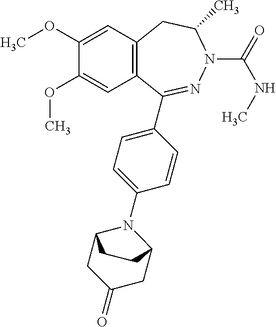 [SMILES: CNC(=O)N1N=C(c2ccc(N3[C@@H]4CC[C@H]3CC(=O)C4)cc2)c2cc(OC)c(OC)cc2C[C@@H]1C] | (4S)-7,8-dimethoxy-N,4- dimethyl-1-[4-(3-oxo-8- azabicyclo[3.2.1]oct-8- yl)phenyl]-4,5-dihydro- 3H-2,3-benzodiazepine- 3-carboxamide | prep. HPLC Method III analyt. HPLC (Method E): Rₜ = 6.63 min |
| 8 | 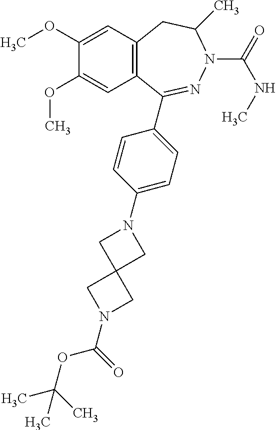 [SMILES: CNC(=O)N1N=C(c2ccc(N3CC4(CN(C(=O)OC(C)(C)C)C4)C3)cc2)c2cc(OC)c(OC)cc2CC1C] | tert-butyl 6-{4-[(±)-7,8- dimethoxy-4-methyl-3- (methylcarbamoyl)-4,5- dihydro-3H-2,3- benzodiazepine-1- yl]phenyl}-2,6- diazaspiro[3.3]heptane-2- carboxylate | ¹H-NMR (400 MHz, CDCl₃): δ = 1.16 (d, 3H), 1.47 (s, 9H), 2.73 (dd, 1H), 2.87 (d, 3H), 2.92 (dd, 1H), 3.72 (s, 3H), 3.96 (s, 3H), 4.08 (s, br, 4H), 4.14 (s, br, 4H), 5.28-5.33 (m, 1H), 6.05 (m, 1H), 6.45 (s, 1H), 6.47 (s, 1H), 6.64 (s, 1H), 6.78 (s, 1H), 7.51 (s, 1H),7.53 (s, 1H). LCMS (method 2): Rₜ = 1.31 min; m/z = 550 (M + H)⁺ |
| 8.1 | 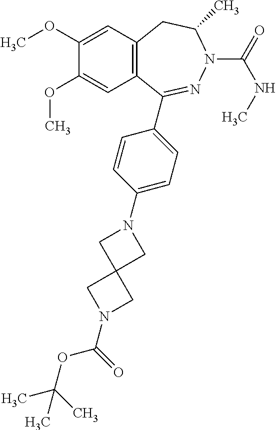 [SMILES: CNC(=O)N1N=C(c2ccc(N3CC4(CN(C(=O)OC(C)(C)C)C4)C3)cc2)c2cc(OC)c(OC)cc2C[C@@H]1C] | tert-butyl 6-{4-[(4S)-7,8- dimethoxy-4-methyl-3- (methylcarbamoyl)-4,5- dihydro-3H-2,3- benzodiazepine-1- yl)phenyl}-2,6- diazaspiro[3.3]heptane-2- carboxylate | LCMS (method 2): Rₜ = 1.31 min; m/z = 550 (M + H)⁺ prep. HPLC Method IV analyt. HPLC (Method D): Rₜ = 3.02 min [α]_{D}²⁰ = 298.5° (c = 1.00; methanol) |
| 8.2 | 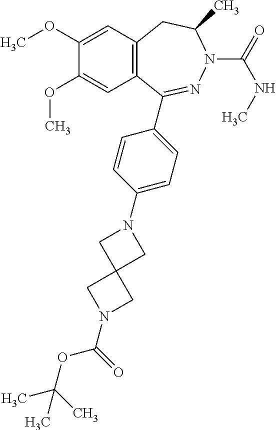 [SMILES: CNC(=O)N1N=C(c2ccc(N3CC4(CN(C(=O)OC(C)(C)C)C4)C3)cc2)c2cc(OC)c(OC)cc2C[C@H]1C] | tert-butyl 6-{4-[(4R)-7,8- dimethoxy-4-methyl-3- (melhylcarbamoyl)-4,5- dihydro-3H-2,3- benzodiazepine-1- yl]phenyl}-2,6- diazaspiro[3.3]heptane-2- carboxylate | LCMS (method 2): Rₜ = 1.31 min; m/z = 550 (M + H)⁺ prep. HPLC Method IV analyt. HPLC (Method D): Rₜ = 2.12 min [α]_{D}²⁰ = −310° (c = 1.00; methanol) |
| 9 | 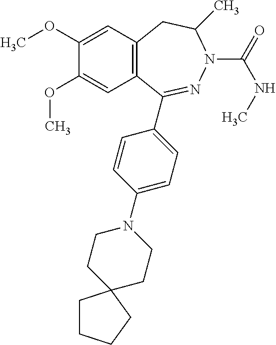 [SMILES: CNC(=O)N1N=C(c2ccc(N3CCC4(CCCC4)CC3)cc2)c2cc(OC)c(OC)cc2CC1C] | (±)-1-[4-(8- azaspiro[4.5]dec-8- yl)phenyl]-7,8- dimethoxy-N,4-dimethyl- 4,5-dihydro-3H-2,3- benzodiazepine-3- carboxamide | ¹H-NMR (400 MHz, CDCl₃): δ = 1.16 (d, 3H), 1.51 (t,4H), 1.61-1.72 (m, 8H), 2.73 (dd, 1H), 2.88 (d, 3H), 2.94 (dd, 1H), 3.31 (m, 4H), 3.73 (s, 3H), 3.96 (s, 3H), 5.22-5.33 (m, 1H), 6.05 (m, 1H), 6.68 (s, 1H), 6.78 (s, 1H), 6.93 (d, 2H), 7.51 (d, 2H). LCMS (method 2): Rₜ = 1.51 min; m/z = 491 (M + H)⁺ |
| 9.1 | 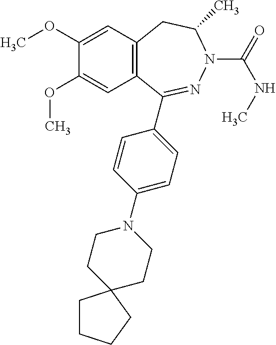 [SMILES: CNC(=O)N1N=C(c2ccc(N3CCC4(CCCC4)CC3)cc2)c2cc(OC)c(OC)cc2C[C@@H]1C] | (4S)-1-[4-(8- azaspiro[4.5]dec-8- yl)phenyl]-7,8- dimethoxy-N,4-dimethyl- 4,5-dihydro-3H-2,3- benzodiazepine-3- carboxamide | prep. HPLC Method VII analyt. HPLC (Method H): Rₜ = 6.53 min [α]_{D}²⁰ = 334.2° +/− 0.30° (c = 1.00; methanol LCMS (method 1): Rₜ = 1.50 min; m/z = 491 (M + H)⁺ |
| 9.2 | 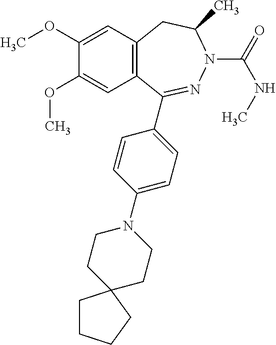 [SMILES: CNC(=O)N1N=C(c2ccc(N3CCC4(CCCC4)CC3)cc2)c2cc(OC)c(OC)cc2C[C@H]1C] | (4R)-1-[4-(8- azaspiro[4.5]dec-8- yl)phenyl]-7,8- dimethoxy-N,4-dimethyl- 4,5-dihydro-3H-2,3- benzodiazepine-3- carboxamide | prep. HPLC Method VII analyt. HPLC (Method H): Rₜ = 4.58 min [α]_{D}²⁰ = −369.0° +/− 0.30° (c = 1.00; methanol LCMS (method 1): Rₜ = 1.50 min; m/z = 491 (M + H)⁺ |
| 10 | 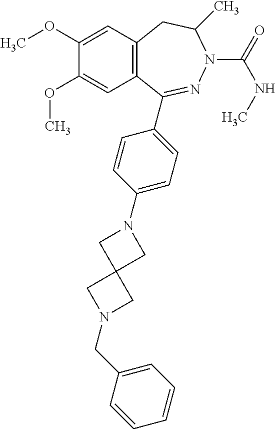 [SMILES: CNC(=O)N1N=C(c2ccc(N3CC4(CN(Cc5ccccc5)C4)C3)cc2)c2cc(OC)c(OC)cc2CC1C] | (±)-1-[4-(6-benzyl-2,6- diazaspiro[3.3]hept-2- yl)phenyl]-7,8- dimethoxy-N,4-dimethyl- 4,5-dihydro-3H-2,3- benzodiazepine-3- carboxamide | ¹H-NMR (400 MHz, CDCl₃): δ = 1.15 (d, 3H), 2.71 (dd, 1H), 2.87 (d, 3H), 2.91 (dd, 1H), 3.44 (s, 4H), 3.63 (s, 2H), 3.72 (s, 3H), 3.95 (s, 3H), 4.04 (s, br, 4H), 5.21-5.33 (m, 1H), 5.98- 6.06 (m, 1H), 6.44 (d, 2H), 6.64 (s, 1H), 6.77 (s, 1H), 7.30-7.40 (m, 5H), 7.48 (d, 2H). LCMS (method 2): Rₜ = 0.84 min; m/z = 540 (M + H)⁺ |
| 11 | 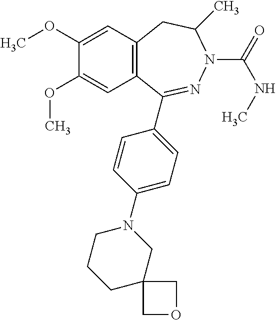 [SMILES: CNC(=O)N1N=C(c2ccc(N3CCCC4(COC4)C3)cc2)c2cc(OC)c(OC)cc2CC1C] | (±)-7,8-dimethoxy-N,4- dimethyl-1-[4-(2-oxa-6- azaspiro[3.5]non-6- yl)phenyl]-4,5-dihydro- 3H-2,3-benzodiazepine- 3-carboxamide | ¹H-NMR (400 MHz, CDCl₃): δ = 1.13 (d, 3H), 1.67-1.76 (m, 2H), 1.88 (t, 2H), 2.75 (dd, 1H), 2.89 (d, 3H), 2.98 (dd, 1H), 3.17 (t, 2H), 3.47 (s, 2H), 3.73 (s, 3H), 3.96 (s, 3H), 4.48 (dd, 4H), 5.27-5.38 (m, 1H), 6.12-6.19 (m, 1H), 6.67 (s, 1H), 6.77 (s, 1H), 6.99 (d, 2H), 7.52 (d, 2H). LCMS (method 2): Rₜ = 1.13 min; m/z = 479 (M + H)⁺ |
| 12 | 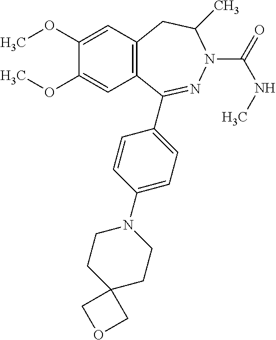 [SMILES: CNC(=O)N1N=C(c2ccc(N3CCC4(CC3)COC4)cc2)c2cc(OC)c(OC)cc2CC1C] | (±)-7,8-dimethoxy-N,4- dimethyl-1-[4-(2-oxa-7- azaspiro[3.5]non-7- yl)phenyl]-4,5-dihydro- 3H-2,3-benzodiazepine- 3-carboxamide | ¹H-NMR (400 MHz, CDCl₃): δ = 1.13 (d, 3H), 2.01-2.09 (m, 4H), 2.74 (dd, 1H), 2.88 (d, 3H), 2.96 (dd, 1H), 3.22-3.29 (t, 4H), 3.72 (s, 3H), 3.96 (s, 3H), 4.51 (s, 4H), 5.26-5.36 (m, 1H), 6.09-6.16 (m, 1H), 6.66 (s, 1H), 6.77 (s, 1H), 6.93 (d, 2H), 7.50 (d, 2H). LCMS (method 2): Rₜ = 1.06 min; m/z = 479 (M + H)⁺ |

Analogously to Example 3.1, Example 30A or Example 32A and the appropriate commercially available amines gave the following exemplary compounds:

| No | Structure | Name | Analytical data |
|---|---|---|---|
| 13 | 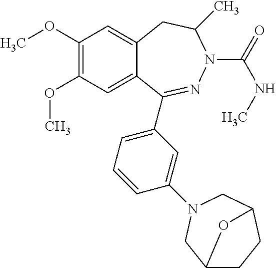 [SMILES: CNC(=O)N1N=C(c2cccc(N3CC4CCC(C3)O4)c2)c2cc(OC)c(OC)cc2CC1C] | (±)-7,8-dimethoxy-N,4- dimethyl-1-[3-(8-oxa-3- azabicyclo[3.2.1]oct-3- yl)phenyl]-4,5-dihydro- 3H-2,3-benzodiazepine- 3-carboxamide | ¹H-NMR (400 MHz, CDCl₃): δ = 1.00 (d, 3H), 1.94 (m, 4H), 2.83 (dd, 1H), 2.86 (d, 3H), 3.01 (m, 2H), 3.08 (dd, 1H), 3.31 (m, 2H), 3.63 (s, 3H), 3.93 (s, 3H), 4.47 (sbr, 2H), 5.43 (m, 1H), 6.47 (m, 1H), 6.63 (s, 1H), 6.71 (s, 1H), 6.85 (dbr, 1H), 6.90 (sbr, 1H), 6.96 (dbr, 1H),7.27 (dd, 1H). LCMS (method 3): Rₜ = 1.21 min; m/z = 465 (M + H)⁺ |
| 14 | 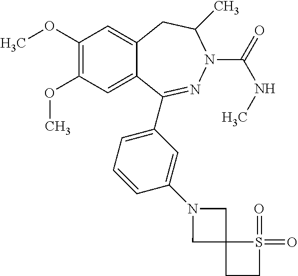 [SMILES: CNC(=O)N1N=C(c2cccc(N3CC4(CCS4(=O)=O)C3)c2)c2cc(OC)c(OC)cc2CC1C] | 1-[3-(1,1-dioxido-1- thia-6-azaspiro[3.3]hept- 6-yl)phenyl]-7,8- dimethoxy-N,4-dimethyl- 4,5-dihydro-3H-2,3- benzodiazepine-3- carboxamide | ¹H-NMR (400 MHz, CDCl₃): δ = 1.00 (d, 3H), 2.43 (m, 2H), 2.83 (dd, 1H), 2.88 (d, 3H), 3.10 (dd, 1H), 3.67 (s, 3H), 3.94 (s, 3H), 4.03 (m, 2H), 4.06 (m, 2H), 4.57 (m, 2H), 5.45 (m, 1H), 6.46 (m, 1H), 6.56 (dd, 1H), 6.59 (s, 1H), 6.62 (dd, 1H), 6.71 (s, 1H), 6.94 (dbr, 1H), 7.27 (dd, 1H). LCMS (method 3): Rₜ = 1.07 min; m/z = 499 (M + H)⁺ |
| 15 | 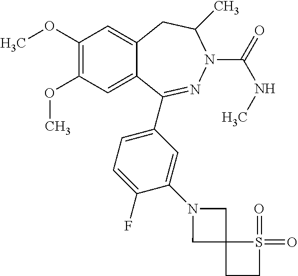 [SMILES: CNC(=O)N1N=C(c2ccc(F)c(N3CC4(CCS4(=O)=O)C3)c2)c2cc(OC)c(OC)cc2CC1C] | (±)-1-[3-(1,1-dioxido-1- thia-6-azaspiro[3.3]hept- 6-yl)-4-fluorophenyl]- 7,8-dimethoxy-N,4- dimethyl-4,5-dihydro-3H- 2,3-benzodiazepine-3- carboxamide | ¹H-NMR (400 MHz, CDCl₃): δ = 1.00 (d, 3H), 2.43 (m, 2H), 2.82 (dd, 1H), 2.88 (d, 3H), 3.07 (dd, 1H), 3.69 (s, 3H), 3.94 (s, 3H), 4.05 (m, 2H), 4.13 (m, 2H), 4.66 (m, 2H), 5.43 (m, 1H), 6.38 (m, 1H), 6.56 (s, 1H), 6.65 (dd, 1H), 6.72 (s, 1H), 6.89 (ddd, 1H), 6.99 (dd, 1H). LCMS (method 3): Rₜ = 1.10 min; m/z = 517 (M + H)⁺ |

### Example 17

### (±)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide

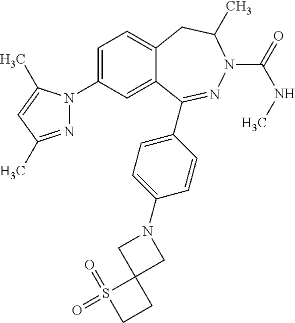 [SMILES: CNC(=O)N1N=C(c2ccc(N3CC4(CCS4(=O)=O)C3)cc2)c2cc(-n3nc(C)cc3C)ccc2CC1C]

The compound was obtained analogously to Example 3.1 from Example 36A in a yield of 77%.

LCMS (method 1): Rₜ=1.15 min; m/z=533 (M+H)⁺

¹H-NMR (300 MHz, DMSO-d₆): δ=1.07 (d, 3H), 2.10 (s, 3H), 2.25 (s, 3H), 2.34-2.43 (m, 2H), 2.48-2.57 (m, 1H), 2.62 (d, 3H), 2.99 (dd, 1H), 4.04-4.17 (m, 4H), 4.39 (dd, 2H), 4.87-4.99 (m, 1H), 6.02 (s, 1H), 6.40 (q, 1H), 6.58 (d, 2H), 7.07 (d, 1H), 7.47-7.56 (m, 2H), 7.62 (d, 2H).

### Enantiomer Separation:

270 mg of (±)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide were separated by preparative HPLC using the following method: system: Sepiatec: Prep SFC100; column: Chiralpak IA 5 μm 250×20 mm; mobile phase: CO₂/methanol 7/3; flow rate: 80 ml/min; pressure (outlet): 150 bar; temperature: 40° C.; detection: UV 254 nm.

### Example 17.1

### (4R)-8-(3,5-Dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide

56 mg, HPLC (Method C): Rₜ=1.95 min, purity 99%

### Example 17.2

### (4S)-8-(3,5-Dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide

69 mg, HPLC (Method C): Rₜ=2.62 min, purity 95.1%

Analogously to Example 17, Example 34A and the appropriate commercially available amine (CAS-No. 1499162-59-2), optionally followed by enantiomer separation using the preparative HPLC method indicated below, gave the following exemplary compounds:

| No | Structure | Name | Analytical data |
|---|---|---|---|
| 18 | 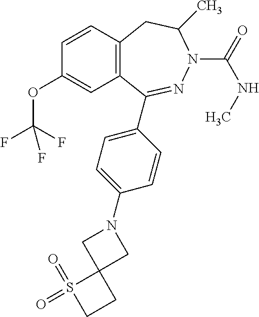 [SMILES: CNC(=O)N1N=C(c2ccc(N3CC4(CCS4(=O)=O)C3)cc2)c2cc(OC(F)(F)F)ccc2CC1C] | (±)-1-[4-(1,1-dioxido-1- thia-6-azaspiro[3.3]hept- 6-yl)phenyl]-N,4- dimethyl-8- (trifluoromethoxy)-4,5- dihydro-3H-2,3- benzodiazepine-3- carboxamide | ¹H-NMR (400 MHz, CDCl₃): δ = 1.06 (d, 3H), 2.45 (m, 2H), 2.81 (dd, 1H), 2.87 (d, 3H), 3.02 (dd, 1H), 4.07 (m, 4H), 4.64 (m, 2H), 5.31 (m, 1H), 6.14 (q, 1H), 6.99 (sbr, 1H), 7.19 (dbr, 2H), 7.27 (d, 1H), 7.44 (d, 2H). LCMS (method 2): Rₜ = 1.23 min; m/z = 523 (M + H)⁺ |
| 18.1 | 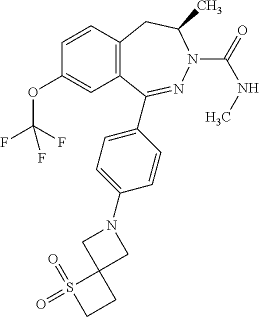 [SMILES: CNC(=O)N1N=C(c2ccc(N3CC4(CCS4(=O)=O)C3)cc2)c2cc(OC(F)(F)F)ccc2C[C@H]1C] | (4R)-1-[4-(1,1-dioxido-1- thia-6-azaspiro[3.3]hept- 6-yl)phenyl]-N,4- dimethyl-8- (trifluoromethoxy)-4,5- dihydro-3H-2,3- benzodiazepine-3- carboxamide | prep. HPLC Method VI analyt. HPLC (Method G): Rₜ = 4.21 min [α]_{D}²⁰ = −401.1° (c = 1.00; methanol) |
| 18.2 | 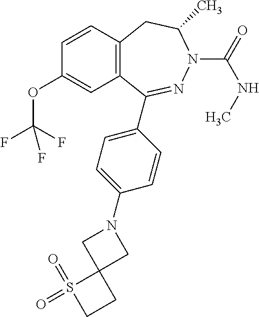 [SMILES: CNC(=O)N1N=C(c2ccc(N3CC4(CCS4(=O)=O)C3)cc2)c2cc(OC(F)(F)F)ccc2C[C@@H]1C] | (4S)-1-[4-(1,1-dioxido-1- thia-6-azaspiro[3.3]hept- 6-yl)phenyl]-N,4- dimethyl-8- (trifluoromethoxy)-4,5- dihydro-3H-2,3- benzodiazepine-3- carboxamide | prep. HPLC Method VI analyt. HPLC (Method G): Rₜ = 5.29 min [α]_{D}²⁰ = 394.3° (c = 1.00; methanol) |

### Example 19

### [1S-(1R*,4R*)]-1-[4-(2,5-Diazabicyclo[2.2.1]hept-2-yl)phenyl]-7,8-dimethoxy-4,5-dihydro-N,4-dimethyl-3H-2,3-benzodiazepine-3-carboxamide

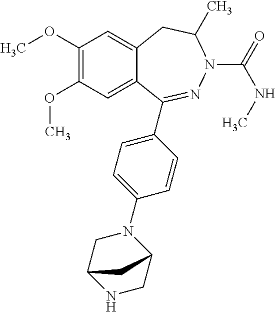 [SMILES: CNC(=O)N1N=C(c2ccc(N3C[C@@H]4C[C@H]3CN4)cc2)c2cc(OC)c(OC)cc2CC1C]

509 mg (926 μmol) of tert-butyl [1S-(1R*,4R*)]-5-{4-[7,8-dimethoxy-4-methyl-3-(methylcarbamoyl)-4,5-dihydro-3H-2,3-benzodiazepin-1-yl]phenyl}-2,5-diazabicyclo[2.2.1]heptane-2-carboxylate were prepared analogously to Example 3.1 from Example 30A using the commercially available tert-butyl (1S,4S)-2,5-diazabicyclo[2.2.1]heptane-2-carboxylate (CAS[113451-59-5]). Analytical data for tert-butyl [1S-(1R*,4R*)]-5-{4-[7,8-dimethoxy-4-methyl-3-(methylcarbamoyl)-4,5-dihydro-3H-2,3-benzodiazepin-1-yl]phenyl}-2,5-diazabicyclo[2.2.1]heptane-2-carboxylate:

¹H-NMR (300 MHz, CDCl₃): δ=1.16 (m, 3H), 1.41/1.46 (s, 9H), 2.00 (m, 2H), 2.69 (dd, 1H), 2.84 (d, 3H), 2.85 (m, 1H), 3.16-3.66 (m, 4H), 3.72/3.74 (s, 3H), 3.93 (s, 3H), 4.47 (s, 1H), 4.53/4.67 (s, 1H), 5.22 (m, 1H), 5.94 (m, 1H), 6.55 (m, 2H), 6.67 (s, 1H), 6.76 (s, 1H), 7.52 (m, 2H).

LCMS (method 3): Rₜ=1.26 min; m/z=550 (M+H)⁺

These were initially charged in 15 ml of dichloromethane and, at 0° C., 713 μl (9.26 mmol) of trifluoroacetic acid were added and stirring was continued at RT for 20 h. The mixture was carefully added to 2 M aqueous sodium hydroxide solution and extracted with dichloromethane. The combined organic phases were dried with sodium sulphate and the solvents were removed on a rotary evaporator. This gave 346 mg (82% of theory) of the desired product as a yellowish solid.

LCMS (method 2): Rₜ=0.66 min; m/z=450 (M+H)⁺

¹H-NMR (300 MHz, CDCl₃): δ=1.16 (d, 3H), 1.90 (dbr, 1H), 1.98 (dbr, 1H), 2.68 (dd, 1H), 2.84 (d, 3H), 2.87 (m, 1H), 3.11 (dd, 1H), 3.12 (m, 1H), 3.68 (dbr, 1H), 3.72 (s, 3H), 3.88 (s, 1H), 3.93 (s, 3H), 4.39 (s, 1H), 5.21 (m, 1H), 5.91 (m, 1H), 6.55 (d, 2H), 6.65 (s, 1H), 6.76 (s, 1H), 7.50 (m, 2H).

Analogously to Example 19, Example 32A was used to prepare, by a cross-coupling reaction, tert-butyl [1S-(1R*,4R*)]-5-{5-[7,8-dimethoxy-4-methyl-3-(methylcarbamoyl)-4,5-dihydro-3H-2,3-benzodiazepin-1-yl]-2-fluorophenyl}-2,5-diazabicyclo[2.2.1]heptane-2-carboxylate. Analytical data:

¹H-NMR (300 MHz, CDCl₃): δ=1.00 (m, 3H), 1.45 (m, 9H), 1.94 (m, 2H), 2.82 (dd, 1H), 2.87 (d, 3H), 3.07 (dd, 1H), 3.17-3.78 (m, 7H), 3.93 (s, 3H), 4.47 (m, 1H), 4.58 (m, 1H), 5.42 (m, 1H), 6.43 (m, 1H), 6.62 (m, 1H), 6.71 (s, 1H), 6.74 (m, 1H), 6.81 (m, 1H), 6.99 (m, 1H).

LCMS (method 3): Rₜ=1.39 min; m/z=568 (M+H)⁺

Subsequent deprotection gave the following exemplary compound.

| No | Structure | Name | Analytical data |
|---|---|---|---|
| 20 | 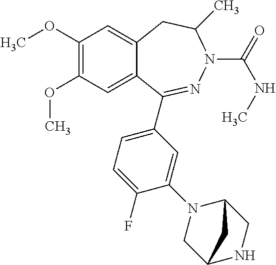 [SMILES: CNC(=O)N1N=C(c2ccc(F)c(N3C[C@@H]4C[C@H]3CN4)c2)c2cc(OC)c(OC)cc2CC1C] | [1S-(1R*,4R*)]-1-[3-(2,5- diazabicyclo[2.2.1]hept- 2-yl)-4-fluorophenyl]- 7,8-dimethoxy-4,5- dihydro-N,4-dimethyl- 3H-2,3-benzodiazepine- 3-carboxamide | ¹H-NMR (300 MHz, CDCl₃): δ = 1.01/1.02 (d, 3H), 1.82 (dbr, 1H), 1.96 (dbr, 1H), 2.84 (d, 3H), 2.89/2.90 (m, 1H), 3.08 (dd, 1H), 3.09 (m, 2H), 3.23 (dbr, 1H), 3.81 (m, 1H), 3.71 (s, 3H), 3.78 (sbr, 1H), 3.96 (s,3H), 4.37/4.43 (s, 1H), 5.44 (m, 1H), 6.45 (m, 1H), 6.63/6.65 (s, 1H), 6.73/6.74 (s, 1H), 6.74 (m, 1H), 6.79 (m, 1H), 7.01 (m, 1H). LCMS (method 3): Rₜ = 0.80 min; m/z = 468 (M + H)⁺ |

General Suzuki coupling procedure for the preparation of Examples 21-27:

2.61 mmol of (±)-1-(4-bromophenyl)-7,8-dimethoxy-N,4-dimethyl-4,5-dihydro-3H-2,3-benzodiazepine-3-carboxamide (Example 30A) were dissolved in 18 ml of 1,4-dioxane, and 6.61 mmol of the appropriate boronic acid, 2.90 ml of 1.5 M aqueous potassium carbonate solution and 0.44 mmol of dichloro[1,1′-bis(diphenylphosphino)ferrocene]palladium(II) (complex with CH₂Cl₂, CAS [95464-05-4]) were added. The mixture was irradiated in the microwave at 130° C. for 15 min and subsequently concentrated to dryness on a rotary evaporator. The residue was purified by preparative RP-HPLC.

| No | Structure | Name | Rₜ | m/z |
|---|---|---|---|---|
| 21 | 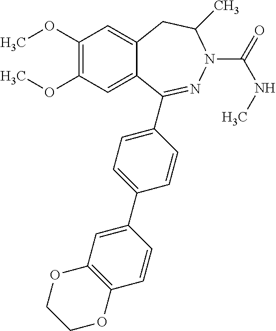 [SMILES: CNC(=O)N1N=C(c2ccc(-c3ccc4c(c3)OCCO4)cc2)c2cc(OC)c(OC)cc2CC1C] | (±)-1-[4-(2,3-dihydro-1,4- benzodioxin-6-yl)phenyl]-7,8- dimethoxy-N,4-dimethyl-4,5- dihydro-3H-2,3- benzodiazepine-3- carboxamide | | |
| 22 | 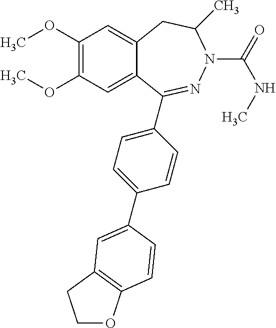 [SMILES: CNC(=O)N1N=C(c2ccc(-c3ccc4c(c3)CCO4)cc2)c2cc(OC)c(OC)cc2CC1C] | (±)-1-[4-(2,3-dihydro-1- benzofuran-5-yl)phenyl]-7,8- dimethoxy-N,4-dimethyl-4,5- dihydro-3H-2,3- benzodiazepine-3- carboxamide | | |
| 23 | 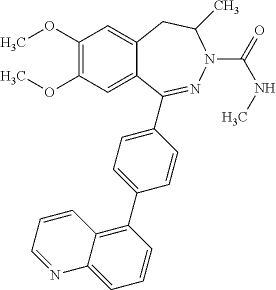 [SMILES: CNC(=O)N1N=C(c2ccc(-c3cccc4ncccc34)cc2)c2cc(OC)c(OC)cc2CC1C] | (±)-1-[4-(quinolin-5- yl)phenyl]-7,8-dimethoxy-N,4- dimethyl-4,5-dihydro-3H-2,3- benzodiazepine-3- carboxamide | | |
| 24 | 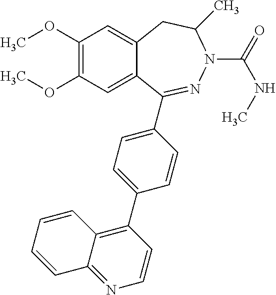 [SMILES: CNC(=O)N1N=C(c2ccc(-c3ccnc4ccccc34)cc2)c2cc(OC)c(OC)cc2CC1C] | (±)-1-[4-(quinolin-4- yl)phenyl]-7,8-dimethoxy-N,4- dimethyl-4,5-dihydro-3H-2,3- benzodiazepine-3- carboxamide | | |
| 25 | 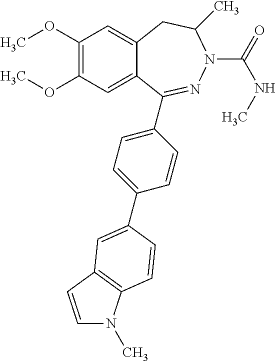 [SMILES: CNC(=O)N1N=C(c2ccc(-c3ccc4c(ccn4C)c3)cc2)c2cc(OC)c(OC)cc2CC1C] | (±)-7,8-dimethoxy-N,4- dimethyl-1-[4-(1-methyl-1H- indol-5-yl)phenyl]-4,5- dihydro-3H-2,3- benzodiazepine-3- carboxamide | | |
| 26 | 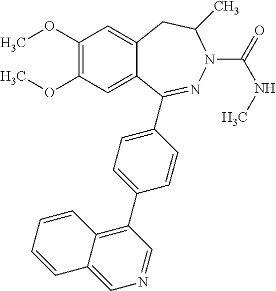 [SMILES: CNC(=O)N1N=C(c2ccc(-c3cncc4ccccc34)cc2)c2cc(OC)c(OC)cc2CC1C] | (±)-1-[4-(isoquinolin-4- yl)phenyl]-7,8-dimethoxy-N,4- dimethyl-4,5-dihydro-3H-2,3- benzodiazepine-3- carboxamide | | |
| 27 | 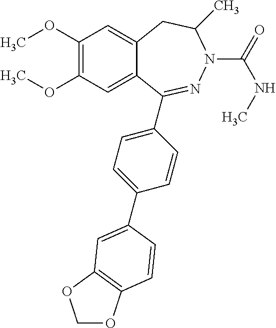 [SMILES: CNC(=O)N1N=C(c2ccc(-c3ccc4c(c3)OCO4)cc2)c2cc(OC)c(OC)cc2CC1C] | (±)-1-[4-(1,3-benzodioxol-5- yl)phenyl]-7,8-dimethoxy-N,4- dimethyl-4,5-dihydro-3H-2,3- benzodiazepine-3- carboxamide | | |

### Example 28

### (±)-7,8-Dimethoxy-N,4-dimethyl-1-[4-(3-oxo-2,8-diazaspiro[4.5]dec-8-yl)phenyl]-4,5-dihydro-3H-2,3-benzodiazepine-3-carboxamide

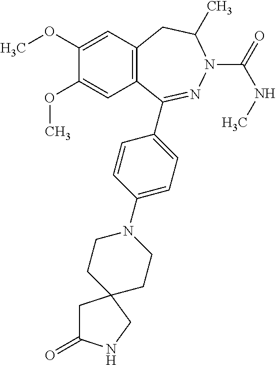 [SMILES: CNC(=O)N1N=C(c2ccc(N3CCC4(CC3)CNC(=O)C4)cc2)c2cc(OC)c(OC)cc2CC1C]

Under argon, 100 mg (0.231 mmol) of (±)-1-(4-bromophenyl)-7,8-dimethoxy-N,4-dimethyl-4,5-dihydro-3H-2,3-benzodiazepine-3-carboxamide (Example 30A), 31 mg (0.324 mmol) of sodium tert-butoxide and 39 mg (0.254 mmol) of 2,8-diazaspiro[4.5]decan-3-one (CAS[561314-57-6] were initially charged in 4 ml of toluene, and the mixture was degassed by flushing with argon. 9.1 mg (0.012 mmol) of chloro-(2-dicyclohexylphosphino-2,4,6-triisopropyl-1,1-biphenyl)[2-(2-amino-1,1-biphenyl)]palladium(II) (CAS [1310584-14-5]) were then added, and the reaction mixture was degassed again, saturated with argon and then stirred at 110° C. for about 16 hours. After cooling, the mixture was added to sat. sodium bicarbonate solution and extracted with ethyl acetate. The combined organic phases were filtered through a water-separating filter and the solvents were removed on a rotary evaporator. The residue was purified by preparative RP-HPLC. This gave 16 mg (14% of theory) of the desired product as a solid.

LCMS (method 1): Rₜ=0.74 min; m/z=506 (M+H)⁺

¹H-NMR (400 MHz, CDCl₃): δ=1.03 (d, 3H), 1.73 (t, 6H), 2.59 (s, 2H), 2.80-3.0 (m, 4H), 2.91 (d, 3H), 3.10 (dd, 1H), 3.69 (s, 3H), 3.72 (s, 2H), 3.96 (s, 3H), 5.39-5.49 (m, 1H), 6.41-6.49 (m, 1H), 6.63 (s, 1H), 6.75 (s, 1H), 7.54 (d, 2H), 7.68 (d, 2H).

Analogously to Example 28, Example 30A or 30.2A and the appropriate commercially available amines:

| CAS number | Name |
|---|---|
| 220290-68-6 | 2-oxa-6-azaspiro[3.4]octane |
| 5654-83-1 | octahydropyrrolo[1,2-a]pyrazine |
| 1061873-16-2 | 2-methyl-2,8-diazaspiro[4.5]decane |
| 945947-99-9 | 3-oxa-1,8-diazaspiro[4.5]decan-2-one |
| 1357396-60-1 | (3aR,6aS)-2-methyloctahydropyrrolo[3,4-c]pyrrole |
| 1427388-39-3 | 2-thia-6-azaspiro[3.3]heptane 2,2-dioxide |
| | hydrochlorid (1:1) |
| 31560-06-2 | (1S,4S)-2-oxa-5-azabicyclo[2.2.1]heptane |
| | hydrochloride (1:1) |
| 1214875-47-4 | octahydrofuro[3,2-c]pyridine |

gave the following exemplary compounds:

| No | Structure | Name | Analytical data |
|---|---|---|---|
| 29 | 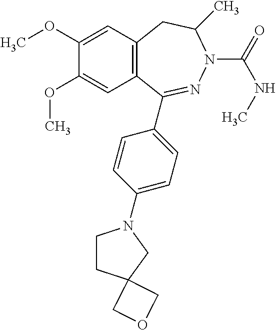 [SMILES: CNC(=O)N1N=C(c2ccc(N3CCC4(COC4)C3)cc2)c2cc(OC)c(OC)cc2CC1C] | (±)-7,8-dimethoxy-N,4- dimethyl-1-[4-(2-oxa-6- azaspiro[3.4]oct-6- yl)phenyl]-4,5-dihydro- 3H-2,3-benzodiazepine- 3-carboxamide | ¹H-NMR (400 MHz, CDCl₃): δ = 1.19 (d, 3H), 2.37 (t, 2H), 2.71 (dd, 1H), 2.88 (d, 3H), 2.89-2.94 (m, 1H), 3.42 (t, 2H), 3.65 (s, 2H), 3.74 (s, 3H), 3.96 (s, 3H), 4.69 (d, 2H), 4.75 (d, 2H), 5.19-5.30 (m, 1H), 5.92-5.99 (m, 1H), 6.57 (d, 2H), 6.68 (s, 1H), 6.79 (s, 1H), 7.55 (d, 2H). LCMS (method 1): Rₜ = 1.05 min; m/z = 465 (M + H)⁺ |
| 30 | 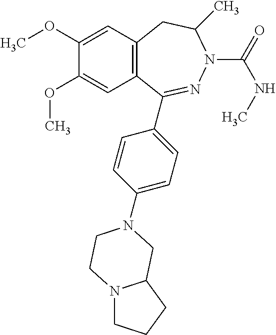 [SMILES: CNC(=O)N1N=C(c2ccc(N3CCN4CCCC4C3)cc2)c2cc(OC)c(OC)cc2CC1C] | (±)-1-[4- (hexahydropyrrolo[1,2- a]pyrazin-2(1H)- yl)phenyl]-7,8- dimethoxy-N,4-dimethyl- 4,5-dihydro-3H-2,3- benzodiazepine-3- carboxamide | ¹H-NMR (400 MHz, CDCl₃): δ = 1.11 (d, 3H), 1.51-1.65 (m, 1H), 1.76- 2.00 (m, 3H), 2.29 (q, 2H), 2.41- 2.53 (m, 1H), 2.65-2.78 (m, 2H), 2.86 (d, 3H), 2.93 (dd, 1H), 3.05 (td, 1H), 3.15-3.25 (m, 2H), 3.67-3.78 (m, 4H), 3.87 (d, 1H), 3.93 (s, 3H), 5.22-5.34 (m, 1H), 6.09 (d, 1H), 6.65 (s, 1H), 6.75 (s, 1H), 6.92 (d, 2H),7.49 (d, 2H). LCMS (method 1): Rₜ = 0.75 min; m/z = 478 (M + H)⁺ |
| 31 | 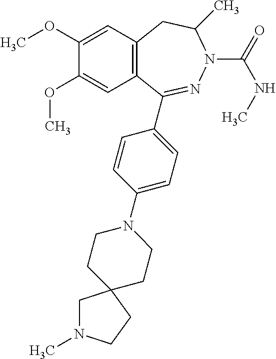 [SMILES: CNC(=O)N1N=C(c2ccc(N3CCC4(CCN(C)C4)CC3)cc2)c2cc(OC)c(OC)cc2CC1C] | (±)-7,8-dimethoxy-N,4- dimethyl-1-[4-(2-methyl- 2,8-diazaspiro[4.5]dec-8- yl)phenyl]-4,5-dihydro- 3H-2,3-benzodiazepine- 3-carboxamide | ¹H-NMR (300 MHz, CDCl₃): δ = 1.15 (d, 3H), 1.68-1.85 (m, 6H), 2.37 (s, 3H), 2.46 (s, 2H), 2.62 (t, 2H), 2.73 (dd, 1H), 2.88 (d, 3H), 2.95 (dd, 1H), 3.22-3.37 (m, 4H), 3.73 (s, 3H), 3.96 (s, 3H), 5.22-5.36 (m, 1H), 6.03-6.13 (m, 1H), 6.67 (s, 1H), 6.77 (s, 1H), 6.94 (d, 2H), 7.51 (d, 2H). LCMS (method 1): Rₜ = 0.78 min; m/z = 506 (M + H)⁺ |
| 32 | 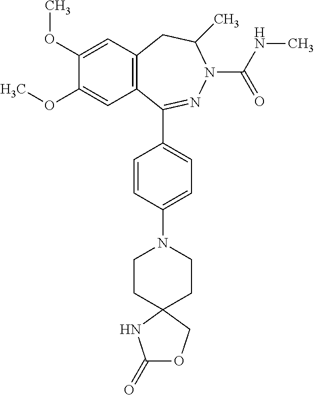 | (±)-7,8-dimethoxy-N,4- dimethyl-2-[4-(2-oxo-3- oxa-1,8- diazaspiro[4.5]dec-8- yl)phenyl]-4,5-dihydro- 3H-2,3-benzodiazepine- 3-carboxamide | ¹H-NMR (300 MHz, CDCl₃): δ = 1.11 (d, 3H), 1.95-2.02 (m, 4H), 2.76 (dd, 1H), 2.89 (d, 3H), 2.99 (dd, 1H), 3.09-3.18 (m, 1H), 3.31-3.39 (m, 4H), 3.72 (s, 3H), 3.96 (s, 3H), 4.26 (s, 2H), 5.28-5.38 (m, 1H), 6.14-6.23 (m, 1H), 6.65 (s, 1H), 6.77 (s, 1H), 6.95 (d, 2H), 7.51 (d, 2H). LCMS (method 2): Rₜ = 0.85 min; m/z = 508 (M + H)⁺ |
| 33 | 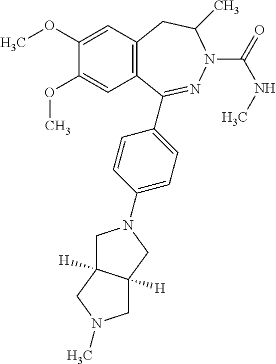 [SMILES: CNC(=O)N1N=C(c2ccc(N3C[C@H]4CN(C)C[C@H]4C3)cc2)c2cc(OC)c(OC)cc2CC1C] | (±)-7,8-dimethoxy-N,4- dimethyl-1-{4- [(3aR,6aS)-5- methylhexahydropyrrolo [3,4-c]pyrrol-2(1H)- yl]phenyl}-4,5-dihydro- 3H-2,3-benzodiazepine- 3-carboxamide | ¹H-NMR (300 MHz, CDCl₃): δ = 1.18 (d, 3H), 2.37 (s, 3H), 2.53 (dd, 2H), 2.71-2.80 (m, 3H), 2.87 (d, 3H), 2.90 (dd, 1H), 3.00-3.10 (m, 2H), 3.30 (m, 2H), 3.53 (m, 2H), 3.74 (s, 3H), 3.96 (s, 3H), 5.18-5.31 (m, 1H), 5.93-6.02 (m, 1H), 6.64 (d, 2H), 6.66 (s, 1H), 6.78 (s, 1H), 7.51 (d, 2H). LCMS (method 1): Rₜ = 0.76 min; m/z = 478 (M + H)⁺ |
| 34 | 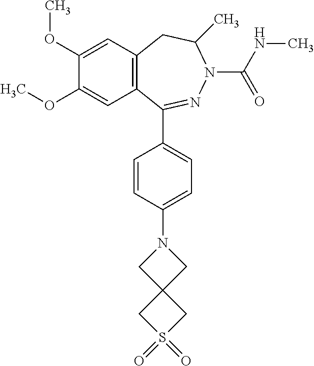 | (±)-1-[4-(2,2-dioxido-2- thia-6-azaspiro[3.3]hept- 6-yl)phenyl]-7,8- dimethoxy-N,4-dimethyl- 4,5-dihydro-3H-2,3- benzodiazepine-3- carboxamide | ¹H-NMR (400 MHz, CDCl₃): δ = 1.12 (d, 3H), 2.75 (dd, 1H), 2.88 (d, 3H), 2.97 (dd, 1H), 3.71 (s, 3H), 3.96 (s, 3H), 4.19 (s, 4H), 4.40 (s, 4H), 5.27- 5.37 (m, 1H), 6.10-6.16 (m, 1H), 6.50 (d, 2H), 6.62 (s, 1H), 6.77 (s, 1H), 7.50 (d, 2H). LCMS (method 2): Rₜ = 0.88 min; m/z = 499 (M + H)⁺ |
| 35 | 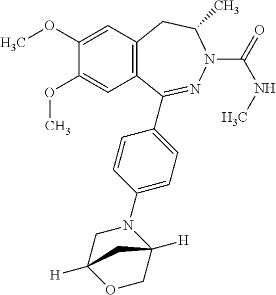 [SMILES: CNC(=O)N1N=C(c2ccc(N3C[C@@H]4C[C@H]3CO4)cc2)c2cc(OC)c(OC)cc2C[C@@H]1C] | (4S)-7,8-dimethoxy-N,4- dimethyl-1-{4-[(1S,4S)-2- oxa-5- azabicyclo[2.2.1]hept-5- yl]phenyl}-4,5-dihydro- 3H-2,3-benzodiazepine- 3-carboxamide | ¹H-NMR (300 MHz, CDCl₃): δ = 1.18 (d, 3H), 1.99-2.10 (m, 2H), 2.72 (dd, 1H), 2.88 (s, 3H), 2.91 (dd, 1H), 3.27 (d, 1H), 3.61 (dd, 1H), 3.75 (s, 3H), 3.88-3.98 (m, 2H), 3.96 (s, 3H), 4.51 (s, 1H), 4.72 (s, 1H), 5.19-5.33 (m, 1H), 5.93-6.02 (m, 1H), 6.60 (d, 2H), 6.68 (s, 1H), 6.79 (s, 1H), 7.53 (d, 2H). LCMS (method 1): Rₜ = 1.02 min; m/z = 451 (M + H)⁺ |
| 36 | 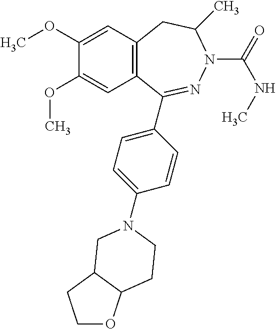 [SMILES: CNC(=O)N1N=C(c2ccc(N3CCC4OCCC4C3)cc2)c2cc(OC)c(OC)cc2CC1C] | (±)-1-[4- (hexahydrofuro[3,2- c]pyridin-5(4H)- yl)phenyl]-7,8- dimethoxy-N,4-dimethyl- 4,5-dihydro-3H-2,3- benzodiazepine-3- carboxamide | ¹H-NMR (300 MHz, CDCl₃): δ = 1.13 (d, 3H), 1.66-1.85 (m, 3H), 2.01 - 2.12 (m, 1H), 2.18-2.25 (m, 1H), 2.67-2.76 (m, 2H), 2.87 (d, 3H), 2.82-2.97 (m, 2H), 3.17-3.25 (m, 1H), 3.71 (s, 3H), 3.95 (s, 3H), 3.97- 4.05 (m, 3H), 4.07-4.14 (m, 1H), 5.23-5.33 (m, 1H), 6.05-6.12 (m, 1H), 6.65 (s, 1H), 6.76 (s, 1H), 6.95 (d, 2H), 7.50 (d, 2H). LCMS (method 1): Rₜ = 1.13 min; m/z = 479 (M + H)⁺ |

### Example 37

### (±)-1-[4-(2-Azaspiro[3.3]hept-2-yl)phenyl]-7,8-dimethoxy-N,4-dimethyl-4,5-dihydro-3H-2,3-benzodiazepine-3-carboxamide

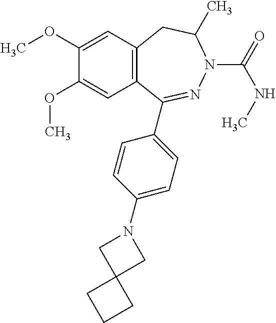 [SMILES: CNC(=O)N1N=C(c2ccc(N3CC4(CCC4)C3)cc2)c2cc(OC)c(OC)cc2CC1C]

Under argon, 100 mg (231 μmol) of (±)-1-(4-bromophenyl)-7,8-dimethoxy-N,4-dimethyl-4,5-dihydro-3H-2,3-benzodiazepine-3-carboxamide (Example 30A), 6.4 mg (7 μmol) of tris(dibenzylideneacetone)dipalladium (CAS [51364-51-3]) and 9.2 mg (23 mol) of 2′-(dicyclohexylphosphino)-N,N-dimethylbiphenyl-2-amine (DavePhos, CAS [213697-53-1]) were initially charged in 2.5 ml of degassed THF in a microwave glass, and the mixture was degassed carefully by introduction of argon. Under argon countercurrent, 31 mg (0.32 mmol) of sodium tert-butoxide and then 124 mg (0.925 mmol) of 2-azaspiro[3.3]heptane hydrochloride (1:1) (CAS[1420271-08-4]) were added. The mixture was degassed again and saturated with argon, the vessel was closed and the mixture was stirred at 85° C. for 30 minutes. After cooling, the mixture was partitioned between water and ethyl acetate and the phases were separated. The solvents were removed on a rotary evaporator and the residue was purified by preparative RP-HPLC. This gave 2.1 mg (2% of theory) of the desired product.

LCMS (method 2): Rₜ=1.35 min; m/z=449.8 (M+H)⁺

¹H-NMR (300 MHz, CDCl₃): δ=1.17 (d, 3H), 1.48-1.98 (m, 2H), 2.25 (t, 4H), 2.71 (dd, 1H), 2.87 (d, 3H), 2.92 (m, 1H), 3.73 (s, 3H), 3.93 (s, 4H), 3.96 (s, 3H), 5.18-5.31 (m, 1H), 5.92-6.00 (m, 1H), 6.43 (d, 2H), 6.65 (s, 1H), 6.78 (s, 1H), 7.49 (d, 2H).

### Example 38

### (±)-7,8-Dimethoxy-N,4-dimethyl-1-[4-(6-methyl-2,6-diazaspiro[3.3]hept-2-yl)phenyl]-4,5-dihydro-3H-2,3-benzodiazepine-3-carboxamide

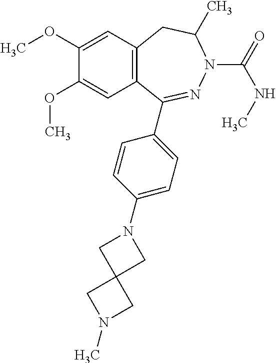 [SMILES: CNC(=O)N1N=C(c2ccc(N3CC4(CN(C)C4)C3)cc2)c2cc(OC)c(OC)cc2CC1C]

Under argon, 200 mg (463 μmol) of (±)-1-(4-bromophenyl)-7,8-dimethoxy-N,4-dimethyl-4,5-dihydro-3H-2,3-benzodiazepine-3-carboxamide (Example 30A), 87.3 mg (278 μmol) of ethanediacid 2-methyl-2,6-diazaspiro[3.3]heptane (1:2) and 62 mg (0.648 mmol) of sodium tert-butoxide were initially charged in 10 ml of toluene. By introduction of argon, the mixture was carefully freed from oxygen, and 1 mg (2 μmol) of 2-[di-(3S,5S,7S)-adamantan-1-ylphosphino]-N,N-dimethylaniline (CAS[1219080-77-9]) and 0.3 mg (1 μmol) of palladium(t-cinnamyl) chloride dimer (CAS [12131-44-1]) were then added. The mixture was degassed again and then heated at 110° C. for 4 h. After cooling, the mixture was partitioned between aqueous saturated sodium bicarbonate solution and ethyl acetate and the phases were separated. The solvents were removed on a rotary evaporator and the residue was purified by preparative RP-HPLC. This gave 6 mg (2% of theory) of the desired product.

LCMS (method 2): Rₜ=0.57 min; m/z=464 (M+H)⁺

¹H-NMR (300 MHz, CDCl₃): δ=1.16 (d, 3H), 2.36 (s, 3H), 2.71 (dd, 1H), 2.87 (d, 3H), 2.91 (dd, 1H), 3.43 (s, br, 4H), 3.72 (s, 3H), 3.95 (s, 3H), 4.03 (s, 4H), 5.19-5.33 (m, 1H), 5.97-6.05 (m, 1H), 6.44 (d, 2H), 6.64 (s, 1H), 6.77 (s, 1H), 7.48 (d, 2H).

Analogously to Example 38, Example 30A and the appropriate commercially available amine gave the following exemplary compound:

| No | Structure | Name | Analytical data |
|---|---|---|---|
| 39 | 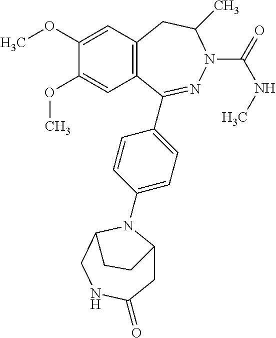 [SMILES: CNC(=O)N1N=C(c2ccc(N3C4CCC3CC(=O)NC4)cc2)c2cc(OC)c(OC)cc2CC1C] | (±)-7,8-Dimethoxy-N,4- dimethyl-1-[4-(4-oxo-3,9- diazabicyclo[4.2.1]non-9- yl)phenyl]-4,5-dihydro- 3H-2,3-benzodiazepine- 3-carboxamide | ¹H-NMR (400 MHz, CDCl₃): δ = 1.02 (d, 3H), 1.90-2.14 (m, 4H), 2.83 (dd, 1H), 2.91 (d, 3H), 2.89-2.96 (m, 3H), 3.10 (dd, 1H), 3.66-3.79 (m, 6H), 3.96 (s, 3H), 4.12 (dd, 1H), 5.39-5.49 (m, 1H), 6.44-6.51 (m, 1H), 6.66 (d, 1H), 6.74 (s, 1H), 7.24 (dd, 2H), 7.54 (d, 2H). LCMS (method 1): Rₜ = 0.72 min; m/z = 492 (M + H)⁺ |

### Example 40

### [1S-(1R*,4R*)]-7,8-Dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide

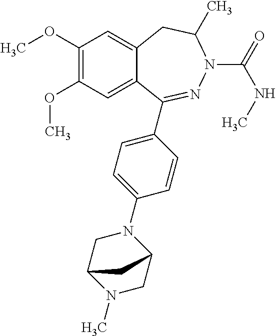 [SMILES: CNC(=O)N1N=C(c2ccc(N3C[C@@H]4C[C@H]3CN4C)cc2)c2cc(OC)c(OC)cc2CC1C]

304 mg (675 μmol) of [1S-(1R*,4R*)]-1-[4-(2,5-diazabicyclo[2.2.1]hept-2-yl)phenyl]-7,8-dimethoxy-4,5-dihydro-N,4-dimethyl-3H-2,3-benzodiazepine-3-carboxamide (Example 19) were dissolved in 10 ml of DMF, and 24 mg (743 μmol) of sodium hydride (60% in mineral oil) were added carefully with ice bath cooling. After 15 min of stirring in the ice bath, 51 μl (810 μmol) of iodomethane were added and the reaction mixture was stirred at room temperature for a further 2 hours. For workup, saturated aqueous sodium bicarbonate solution was added. The mixture was extracted 3× with ethyl acetate, the combined organic phases were washed with sat. sodium chloride solution and dried with sodium sulphate. The solvents were removed on a rotary evaporator and the crude product was purified by preparative HPLC. This gave 143 g (46% of theory) of the desired product as a mixture of epimers.

¹H-NMR (400 MHz, CDCl₃): δ=1.19/1.20 (d, 3H), 1.94 (dbr, 1H), 2.06 (dbr, 1H), 2.44 (s, 3H), 2.71 (m, 1H), 2.89 (m, 3H), 2.87 (d, 3H), 3.00 (dd, 1H), 3.44 (m, 2H), 3.57 (sbr, 1H), 3.75 (s, 3H), 3.96 (s, 3H), 4.32 (sbr, 1H), 5.93 (m, 1H), 6.57 (d, 2H), 6.69/6.68 (s, 1H), 6.79 (s, 1H), 7.52 (d, 2H).

LCMS (method 3): Rₜ=0.68 min; m/z=464 (M+H)⁺

### Epimer Separation:

136 mg of [1S-(1R*,4R*)]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide were separated by preparative HPLC using the following method: system: Sepiatec: Prep SFC100; column: Chiralpak ID 5 μm 250×20 mm; mobile phase: CO₂/ethanol 65/35+0.5% vol. diethylamine; flow rate: 80 ml/min; pressure (outlet): 100 bar; temperature: 40° C.; detection: UV 254 nm.

### Example 40.1

### [1S-[1R*,2(S*),4R*]]-7,8-Dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide

44.5 mg, HPLC (Method J): Rₜ=3.20 min, purity 97.4%

### Example 40.2

### [1S-[1R*,2(R*),4R*]]-7,8-Dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide

42.3 mg, HPLC (Method J): Rₜ=4.76 min, purity 99%

Analogously to Example 40, the following exemplary compounds were prepared from Example 20:

| No | Structure | Name | Analytical data |
|---|---|---|---|
| 41 | 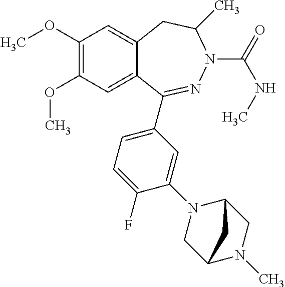 | [1S-(1R*,4R*)]-1-[4- fluoro-3-(5-methyl-2,5- diazabicyclo[2.2.1]hept- 2-yl)phenyl]-7,8- dimethoxy-4,5-dihydro- N,4-dimethyl-3H-2,3- benzodiazepine-3- carboxamide | ¹H-NMR (400 MHz, CDCl₃): δ = 0.99/1.01 (d, 3H), 1.85/1.86 (m, 1H), 1.94 (m, 1H), 2.39 (s, 3H), 2.80 (m, 2H), 2.84 (m, 1H), 2.87 (d, 3H), 3.07/3.08 (dd, 1H), 3.38 (m, 1H), 3.42 (sbr, 1H), 3.53 (m, 1H), 3.68 (s, 3H), 3.93 (s, 3H), 4.27/4.29 (sbr, 1H), 5.41 (m, 1H), 6.40/6.45 (m, 1H), 6.62/6.63 (s, 1H), 6.67-6.83 (m, 2H), 6.71/6.71 (s, 1H), 6.96/7.00 (dd, 1H). LCMS (method 3): Rₜ = 1.14 min; m/z = 482 (M + H)⁺ |
| 41.1 | 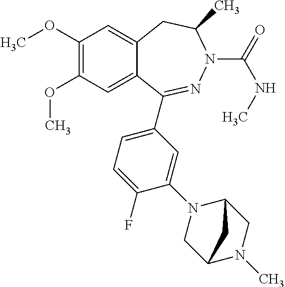 | [1S-(1R*,2(S*),4R*)]-1- [4-fluoro-3-(5-methyl- 2,5- diazabicyclo[2.2.1]hept- 2-yl)phenyl]-7,8- dimethoxy-4,5-dihydro- N,4-dimethyl-3H-2,3- benzodiazepine-3- carboxamide | prep. HPLC Method VIII analyt. HPLC (Method K): Rₜ = 1.18 min [α]_{D}²⁰ = −199.8° (c = 1.00; methanol) |
| 41.2 | 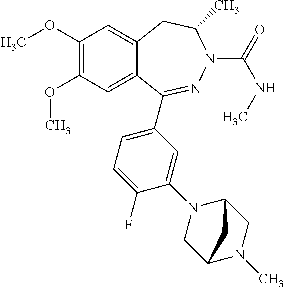 | [1S-(1R*,2(R*),4R*)]-1- [4-fluoro-3-(5-methyl- 2,5- diazabicyclo[2.2.1]hept 2-yl)phenyl]-7,8- dimethoxy-4,5-dihydro- N,4-dimethyl-3H-2,3- benzodiazepine-3- carboxamide | prep. HPLC Method VIII analyt. HPLC (Method K): Rₜ = 1.38 min [α]_{D}²⁰ = 89.1° (c = 1.00; methanol) |

### Biological Efficacy of the Compounds According to the Invention

### 1. Assays

### 1.1 Protein-Protein Interaction Assay

### Binding Assay BRD4/Acetylated Peptide H4 (“PRO”)

To assess the BRD4 binding strength of the substances described in this application, the ability thereof to inhibit the interaction between BRD4 (BD1) and acetylated histone H4 in a dose-dependent manner was quantified.

For this purpose, a time-resolved fluorescence resonance energy transfer (TR-FRET) assay was used, which measures the binding between N-terminally His₆-tagged BRD4 (BD1) (amino acids 67-152, longer constructs also being possible, preferably amino acids 44-168) and a synthetic acetylated histone H4 (Ac—H4) peptide with sequence GRGK(Ac)GGK(Ac)GLGK(Ac)GGAK(Ac)RHGSGSK-biotin. The recombinant BRD4 protein produced in-house according to Filippakopoulos et al., Nature, 2010, 468:1119-1123 was expressed in E. coli and purified by means of (Ni-NTA) affinity and (Sephadex G-75) size exclusion chromatography. The Ac—H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4 solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4 and the substances. Subsequently, 3 μl of a 1.67-fold concentrated solution (in assay buffer) consisting of Ac—H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4/Ac—H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4/Ac—H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4 were present. The IC₅₀ was determined by regression analysis based on a 4-parameter equation (minimum, maximum, IC₅₀, Hill; Y=max+(min−max)/(1+(X/IC₅₀)^{Hill})).

### 1.2 Cell Assays

### Cell Proliferation Assays

In accordance with the invention, the ability of the substances to inhibit cell proliferation was determined. Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells (DSMZ, ACC 554) were sown at a concentration of 4000 cells/well in 100 μl of growth medium (RPMI1640, 10% FCS) on 96-well microtitre plates.

The MV4-11 cells (ATCC, CRL 9591) were sown at a concentration of 5000 cells/well in 100 μl of growth medium (RPMI1640, 10% FCS) on 96-well microtitre plates.

The B16F10 cells (ATCC, CRL-6475) were sown at a concentration of 300-500 cells/well in 100 μl of growth medium (DMEM with phenol red, 10% FCS) on 96-well microtitre plates.

The LOX-IMVI cells (NCI-60) were sown at a concentration of 1000 cells/well in 100 μl of growth medium (RPMI1640, 10% FCS) on 96-well microtitre plates.

The MOLP-8 cells (DSMZ, ACC 569) were sown at a concentration of 4000 cells/well in 100 μl of growth medium (RPMI1640, 20% FCS) on 96-well microtitre plates.

The KMS-12-PE cells (DSMZ, ACC 606) were sown at a concentration of 4000 cells/well in 100 μl of growth medium (RPMI1640, 20% FCS) on 96-well microtitre plates.

The LAPC-4 cells (ATCC, PTA-1441TM) were sown at a concentration of 4000 cells/well in 100 μl of growth medium (RPMI1640, 2 mM L-glutamine, 10% cFCS) on 96-well microtitre plates. One day later, the LAPC-4 cells were treated with 1 nM methyltrienolone and various substance dilutions.

The MDA-MB-231 cells (DSMZ, ACC 732) were sown at a concentration of 4000 cells/well in 100 μl of growth medium (DMEM/Ham's F12 medium, 10% FCS) on 96-well microtitre plates. After overnight incubation at 37° C., the fluorescence values (CI values) were determined. Then the plates were treated with various substance dilutions (1E-5 M, 3E-6 M, 1E-6 M, 3E-7 M, 1E-7 M, 3E-8 M, 1E-8 M) and incubated at 37° C. for 72 (MV4-11, LOX-IMVI cells), 96 (MOLM-13, B16F10, MDA-MB-431 cells), 120 (MOLP-8, KMS-12-PE cells) or 168 (LAPC-4 cells) hours. Subsequently, the fluorescence values were determined (CO values). For the data analysis, the CI values were subtracted from the CO values and the results were compared between cells which had been treated with various dilutions of the substance or only with buffer solution. This was used to calculate the IC50 values (substance concentration required for 50% inhibition of cell proliferation).

The substances were examined in the cell lines of Table 1 which, in an exemplary manner, represent the stated indications:

TABLE 1

| Cell line | Source | Indication |
|---|---|---|
| MOLM-13 | DSMZ | acute myeloid leukaemia |
| MV4-11 | ATCC | acute myeloid leukaemia |
| B16F10 | ATCC | melanoma (BRAF wild-type) |
| LOX IMVI | NCI-60 | melanoma (BRAF mutated) |
| MOLP-8 | DSMZ | multiple myeloma |
| KMS-12-PE | DSMZ | multiple myeloma |
| LAPC-4 | ATCC | prostate cancer |
| MDA-MB-231 | DSMZ | mammary carcinoma |

### 2. Results:

### 2.1 Binding Assay

Table 2 shows the results from the BRD4 (BD1) binding assay.

TABLE 2

| | IC₅₀ (BRD4) |
|---|---|
| Example | (μmol/l) |
| 1 | 0.03 |
| 2 | 0.02 |
| 3 | 0.02 |
| 3.1 | 0.02 |
| 3.2 | 2.58 |
| 4 | 0.03 |
| 4.1 | 1.85 |
| 4.2 | 0.01 |
| 5 | 0.02 |
| 5.1 | 0.02 |
| 5.2 | 1.36 |
| 6 | 0.03 |
| 6.1 | 1.23 |
| 6.2 | 0.02 |
| 8 | 0.14 |
| 8.1 | 0.06 |
| 8.2 | 2.05 |
| 9 | 0.25 |
| 9.1 | 0.2 |
| 9.2 | >20.0 |
| 10 | 0.08 |
| 11 | 0.06 |
| 12 | 0.08 |
| 13 | 0.09 |
| 14 | 0.17 |
| 15 | 0.25 |
| 17 | 0.04 |
| 17.1 | 1.47 |
| 17.2 | 0.01 |
| 18 | 0.04 |
| 18.1 | 11.55 |
| 18.2 | 0.01 |
| 19 | 0.14 |
| 20 | 0.41 |
| 21 | 0.09 |
| 22 | 0.05 |
| 23 | 0.06 |
| 24 | 0.07 |
| 25 | 0.05 |
| 26 | 0.06 |
| 27 | 0.11 |
| 28 | 0.1 |
| 29 | 0.05 |
| 30 | 0.07 |
| 31 | 0.1 |
| 32 | 0.11 |
| 33 | 0.09 |
| 34 | 0.07 |
| 35 | 4.11 |
| 36 | 0.04 |
| 37 | 0.17 |
| 38 | 0.1 |
| 39 | 0.12 |
| 40 | 0.18 |
| 40.1 | 0.07 |
| 40.2 | 0.52 |
| 41 | 0.43 |
| 41.1 | 16.51 |
| 41.2 | 0.38 |

### 2.2 Cell Assays

Tables 3A and 3B show the results of various cell proliferation assays.

TABLE 3A

| | MOLM-13 | MV4-11 | B16F10 | LOX IMVI | MOLP-8 | KMS-12-PE |
|---|---|---|---|---|---|---|
| Example | IC₅₀ (μmol/l) | IC₅₀ (μmol/l) | IC₅₀ (μmol/l) | IC₅₀ (μmol/l) | IC₅₀ (μmol/l) | IC₅₀ (μmol/l) |
| 1 | 0.14 | | | | | |
| 3 | 0.15 | | | | | |
| 3.1 | 0.07 | 0.04 | 0.04 | 0.28 | 0.04 | 0.05 |
| 4 | 0.10 | | | | | |
| 4.1 | 6.09 | | | | | |
| 4.2 | 0.04 | 0.03 | 0.02 | 0.17 | 0.02 | 0.03 |
| 5 | 0.21 | | | | | |
| 5.1 | 0.11 | | | | | |
| 6 | 0.18 | | | | | |
| 6.1 | 0.45 | | | | | |
| 6.2 | 0.05 | | | | | |
| 8 | 0.26 | | 0.18 | | 0.16 | |
| 8.1 | 0.18 | | 0.13 | | 0.10 | |
| 9 | 0.50 | | 0.14 | | 0.46 | |
| 10 | 0.15 | | 0.15 | | 0.08 | |
| 11 | 0.16 | | 0.10 | | 0.10 | |
| 12 | 0.22 | | 0.17 | | 0.14 | |
| 13 | 0.56 | | | | | |
| 14 | 0.33 | | 0.26 | | 0.24 | |
| 15 | 0.60 | | 0.59 | | 0.48 | |
| 17.1 | 6.74 | | 5.59 | | 4.32 | |
| 17.2 | 0.10 | | 0.08 | | 0.06 | |
| 18 | 0.07 | | 0.06 | | 0.06 | |
| 18.2 | 0.03 | | 0.03 | | 0.03 | |
| 19 | 0.17 | | 0.15 | | 0.10 | |
| 20 | 1.07 | | 1.43 | | 0.77 | |
| 21 | 0.71 | | | | | |
| 22 | 0.55 | | | | | |
| 23 | 0.25 | | | | | |
| 24 | 0.39 | | | | | |
| 25 | 0.53 | | | | | |
| 26 | 0.55 | | | | | |
| 27 | 0.48 | | 0.35 | | 0.46 | |
| 29 | 0.22 | | 0.10 | | 0.09 | |
| 30 | 0.20 | | | | 0.09 | |
| 33 | 0.22 | | | | 0.11 | |
| 36 | 0.15 | | 0.07 | | 0.09 | |
| 37 | 0.42 | | 0.42 | | 0.45 | |
| 38 | 0.17 | | 0.40 | | 0.14 | |
| 40 | 0.39 | | 0.45 | | 0.24 | |
| 40.1 | 0.20 | | 0.21 | | 0.10 | |
| 41 | 1.14 | | 1.47 | | 0.69 | |

TABLE 3B

| | LAPC-4 | MDA-MB-231 |
|---|---|---|
| Example | IC₅₀ (μmol/l) | IC₅₀ (μmol/l) |
| 3.1 | 0.02 | 0.08 |
| 4.2 | 0.01 | 0.04 |

1. Compounds of the general formula (I)
in which 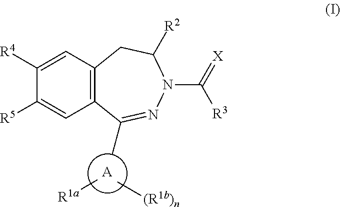
X represents an oxygen or sulphur atom,
A represents a monocyclic heteroaryl ring having 5 or 6 ring atoms,
or
represents a phenyl ring,
R^{1a} represents a spirocycloalkyl, heterospirocycloalkyl, bicycloalkyl, heterobicycloalkyl radical, a bridged cycloalkyl radical or a bridged heterocycloalkyl radical, a naphthyl radical or a bicyclic heteroaryl radical, or a partially saturated bicyclic aryl or heteroaryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
n represents 0, 1 or 2,
R^{1b} represents halogen, hydroxy, cyano, nitro or represents a C₁-C₆-alkyl-, C₁-C₆-alkoxy-, C₁-C₆-alkoxy-C₁-C₆-alkyl-, halo-C₁-C₆-alkyl-, halo-C₁-C₆-alkoxy-, C₃-C₁₀-cycloalkyl radical or a monocyclic heterocyclyl radical having 3 to 8 ring atoms,
R² represents a C₁-C₃-alkyl or trifluoromethyl or a C₃- or C₄-cycloalkyl radical,
R³ represents cyclopropyl-, C₁-C₃-alkyl-, C₁-C₃-alkoxy-, amino-, cyclopropylamino- or C₁-C₃-alkylamino-,
R⁴ and R⁵ independently of one another represent hydrogen, hydroxy, cyano, nitro, amino, aminocarbonyl-, fluorine, chlorine or bromine,
or
represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms where the monocyclic heterocyclyl and heteroaryl radicals mentioned may for their part optionally be monosubstituted by C₁-C₃-alkyl,
or
represent C₃-C₁₀-cycloalkyl- which may optionally be mono- or polysubstituted by identical or different substituents from the group consisting of halogen, amino, hydroxy, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
or
represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy-, C₃-C₁₀-cycloalkyl and a monocyclic heterocyclyl radical having 3 to 8 ring atoms,
or
represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
or
represents phenyl- which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ are presents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₆-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- may optionally be mono- or disubstituted by halogen, C₁-C₃-alkoxy- or C₁-C₃-alkyl-,
R⁹ represents C₁-C₆-alkyl-,
and their polymorphs, enantiomers, diastereomers, racemates, tautomers, solvates, physiologically acceptable salts and solvates.

2. Compounds of the general formula (I) according to claim 1 in which
X represents an oxygen atom,
A represents a monocyclic heteroaryl ring having 6 ring atoms which may contain one or two nitrogen atoms,
or
represents a phenyl ring,
R^{1a} represents a heterospirocycloalkyl, heterobicycloalkyl radical or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
n represents 0, 1 or 2,
R^{1b} represents halogen, hydroxy, cyano, nitro or represents a C₁-C₆-alkyl-, C₁-C₆-alkoxy-, C₁-C₆-alkoxy-C₁-C₆-alkyl-, halo-C₁-C₆-alkyl-, halo-C₁-C₆-alkoxy-, C₃-C₁₀-cycloalkyl radical or a monocyclic heterocyclyl radical having 3 to 8 ring atoms,
R² represents methyl,
R³ represents cyclopropyl-, C₁-C₃-alkyl-, C₁-C₃-alkoxy-, amino-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
or
represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- or amino-C₁-C₆-alkyl-, monocyclic heterocyclyl- having 3 to 8 ring atoms and monocyclic heteroaryl- having 5 or 6 ring atoms where the monocyclic heterocyclyl and heteroaryl radicals mentioned may for their part optionally be monosubstituted by C₁-C₃-alkyl,
or
represent C₃-C₁₀-cycloalkyl- which may optionally be mono- or polysubstituted by identical or different substituents from the group consisting of halogen, amino, hydroxy, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
or
represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy-, C₃-C₁₀-cycloalkyl- and a monocyclic heterocyclyl radical having 3 to 8 ring atoms,
or
represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
or
represents phenyl- which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl, C₁-C₆-alkylaminosulphonyl-, C₁-C₆-alkylamino-C₁-C₆-alkyl, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl, monocyclic heterocyclyl- having 3 to 8 ring atoms or monocyclic heteroaryl- having 5 or 6 ring atoms,
R⁹ represents C₁-C₆-alkyl-,
and their polymorphs, enantiomers, diastereomers, racemates, tautomers, solvates, physiologically acceptable salts and solvates of these salts.

3. Compounds of the general formula (I) according to claim 1 in which
X represents an oxygen atom,
A represents a phenyl ring,
R^{1a} represents a heterospirocycloalkyl radical, heterobicycloalkyl radical or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, C₁-C₆-alkylcarbonylamino-, amino-C₁-C₆-alkyl-, C₁-C₆-alkylamino-C₁-C₆-alkyl-, fluoro-C₁-C₆-alkyl-, fluoro-C₁-C₆-alkoxy-, C₃-C₁₀-cycloalkyl-, phenyl-, halophenyl-, phenyl-C₁-C₆-alkyl-, phenoxy-, pyridinyl-, —C(═O)—NR⁶R⁷, —C(═O)—R⁸, —S(═O)₂—NR⁶R⁷, —S(O)—R⁹, —S(O)₂—R⁹, —NH—S(═O)₂—R⁹ and a monocyclic heterocyclyl radical having 3 to 8 ring atoms,
n represents 0 or 1,
R^{1b} represents halogen, hydroxy, cyano, nitro or represents a C₁-C₃-alkyl-, C₁-C₃-alkoxy-, C₁-C₃-alkoxy-C₁-C₃-alkyl-, fluoro-C₁-C₃-alkyl-, fluoro-C₁-C₃-alkoxy-, C₃-C₇-cycloalkyl radical or a monocyclic heterocyclyl radical having 5 or 6 ring atoms,
R² represents methyl,
R³ represents cyclopropyl-, C₁-C₃-alkyl-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
or
represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
or
represent C₃-C₇-cycloalkyl- which may optionally be mono- or polysubstituted by identical or different substituents from the group consisting of halogen, amino, hydroxy, carboxy, C₁-C₃-alkyl-, C₁-C₃-alkoxy-, C₁-C₃-alkylamino-, amino-C₁-C₃-alkyl-, fluoro-C₁-C₃-alkyl-, fluoro-C₁-C₃-alkoxy- and a monocyclic heterocyclyl radical having 5 or 6 ring atoms,
or
represent monocyclic heteroaryl having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, hydroxy-C₁-C₃-alkyl-, C₁-C₃-alkylamino-, amino-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
or
represent monocyclic heterocyclyl having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, C₁-C₃-alkylamino-, amino-C₁-C₃-alkyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
or
represents phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylamino-, amino-C₁-C₃-alkyl-, C₁-C₃-alkylaminocarbonyl, C₆-C₃-alkylaminosulphonyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, fluoro-C₁-C₃-alkyl-, hydroxy-C₁-C₃-alkyl-, C₃-C₆-cycloalkyl-, phenyl, monocyclic heterocyclyl- having 3 to 8 ring atoms or monocyclic heteroaryl- having 5 or 6 ring atoms,
R⁹ represents C₁-C₆-alkyl-,
and their polymorphs, enantiomers, diastereomers, racemates, tautomers, solvates, physiologically acceptable salts and solvates of these salts.

4. Compounds of the general formula (I) according to claim 1 in which
X represents an oxygen atom,
A represents a phenyl ring,
R^{1a} represents a heterospirocycloalkyl-, heterobicycloalkyl- or a bridged heterocycloalkyl radical, a naphthyl radical or a bicyclic heteroaryl radical, or a partially saturated bicyclic aryl radical where the radicals mentioned may optionally be mono- or polysubstituted by identical or different substituents from the group consisting of halogen, cyano, nitro, hydroxy, amino, oxo, carboxy, C₁-C₄-alkyl-, C₁-C₄-alkoxy-, hydroxy-C₁-C₄-alkyl-, C₁-C₄-alkylamino-, C₁-C₄-alkylcarbonylamino-, amino-C₁-C₄-alkyl-, fluoro-C₁-C₄-alkyl-,
fluoro-C₁-C₄-alkoxy-, C₃-C₈-cycloalkyl-, phenyl-, halophenyl-, phenyl-C₁-C₄-alkyl-, phenoxy-, pyridinyl-, —C(═O)—NR⁶R⁷, —C(═O)—R⁸, —S(═O)₂—NR⁶R⁷, —S(═O)₂—R⁹, —NH—S(═O)₂—R⁹ and a monocyclic heterocyclyl radical having 3 to 8 ring atoms,
n represents 0 or 1,
R^{1b} represents halogen, hydroxy, cyano, nitro or represents a C₁-C₃-alkyl-, C₁-C₃-alkoxy-, C₁-C₃-alkoxy-C₁-C₃-alkyl-, fluoro-C₁-C₃-alkyl-, fluoro-C₁-C₃-alkoxy-, C₃-C₇-cycloalkyl radical or a monocyclic heterocyclyl radical having 5 or 6 ring atoms,
R² represents methyl,
R³ represents cyclopropyl-, C₁-C₃-alkyl-, cyclopropylamino- or C₁-C₃-alkylamino-,
R⁴ and R⁵ independently of one another represent hydrogen, hydroxy, cyano, nitro, amino, aminocarbonyl-, fluorine, chlorine, bromine,
or
represent C₁-C₆-alkyl-, C₁-C₆-alkoxy-, C₁-C₆-alkylamino-, C₁-C₆-alkylcarbonylamino-, C₁-C₆-alkylaminocarbonyl- or C₁-C₆-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
or
represent monocyclic heteroaryl having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, hydroxy-C₁-C₃-alkyl-, C₁-C₃-alkylamino-, amino-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
or
represent monocyclic heterocyclyl having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, C₁-C₃-alkylamino-, amino-C₁-C₃-alkyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
or
represent phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylamino-, amino-C₁-C₃-alkyl-, C₁-C₃-alkylaminocarbonyl, C₁-C₃-alkylaminosulphonyl-, hydroxy-C₁-C₃-alkyl-, fluoro-C₁-C₃-alkyl-, fluoro-C₁-C₃-alkoxy-, C₃-C₇-cycloalkyl- and a monocyclic heterocyclyl radical having 5 or 6 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₄-alkyl-, C₁-C₄-alkoxy-, fluoro-C₁-C₃-alkyl-, hydroxy-C₁-C₃-alkyl-, C₃-C₈-cycloalkyl-, phenyl, monocyclic heterocyclyl- having 3 to 8 ring atoms or monocyclic heteroaryl- having 5 or 6 ring atoms,
R⁹ represents C₁-C₄-alkyl-,
and their polymorphs, enantiomers, diastereomers, racemates, tautomers, solvates, physiologically acceptable salts and solvates of these salts.

5. Compounds of the general formula (I) according to claim 1 in which
X represents an oxygen atom,
A represents a phenyl ring,
R^{1a} represents a heterospirocycloalkyl, heterobicycloalkyl or a bridged heterocycloalkyl radical, a naphthyl radical or a bicyclic heteroaryl radical, or a partially saturated bicyclic aryl radical, where the radicals mentioned may optionally be mono- or polysubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl-, halophenyl-, phenyl-C₁-C₂-alkyl, pyridinyl-, phenoxy- and —C(═O)—R⁸,
n represents 0 or 1,
R^{1b} represents halogen, hydroxy, cyano, or represents a C₁-C₃-alkyl-, C₁-C₃-alkoxy-, fluoro-C₁-C₃-alkyl- or fluoro-C₁-C₃-alkoxy radical,
R² represents methyl,
R³ represents C₁-C₃-alkyl- or C₁-C₃-alkylamino-,
R⁴ and R⁵ independently of one another represent hydrogen, hydroxy, cyano, aminocarbonyl-, fluorine, chlorine, bromine,
or
represent C₁-C₃-alkyl-, C₁-C₄-alkoxy-, C₁-C₆-alkylamino-, C₁-C₄-alkylcarbonylamino-, C₁-C₄-alkylaminocarbonyl- or C₁-C₄-alkylaminosulphonyl- which may optionally be mono- or polysubstituted by identical or different substituents from the group consisting of halogen, amino, hydroxy, carboxy, hydroxy-C₁-C₃-alkyl-, C₁-C₃-alkoxy-, C₁-C₃-alkylamino- and amino-C₁-C₃-alkyl-,
or
represent monocyclic heteroaryl- having 5 or 6 ring atoms which may optionally be mono- or polysubstituted by identical or different substituents from the group consisting of halogen, amino, hydroxy, cyano, nitro, carboxy, C₁-C₃-alkyl-, C₁-C₃-alkoxy-, fluoro-C₁-C₃-alkyl- and fluoro-C₁-C₃-alkoxy-,
or
represent monocyclic heterocyclyl- having 3 to 8 ring atoms which may optionally be mono- or polysubstituted by identical or different substituents from the group consisting of halogen, amino, hydroxy, cyano, oxo, carboxy, C₁-C₃-alkyl-, C₁-C₃-alkoxy-, fluoro-C₁-C₃-alkyl- and fluoro-C₁-C₃-alkoxy-,
or
represents phenyl- which may optionally be mono- or polysubstituted by identical or different substituents from the group consisting of halogen, amino, hydroxy, cyano, nitro, carboxy, C₁-C₃-alkyl-, C₁-C₃-alkoxy-, C₁-C₃-alkylaminocarbonyl, C₁-C₃-alkylaminosulphonyl-, fluoro-C₁-C₃-alkyl- and fluoro-C₁-C₃-alkoxy-,
R⁸ represents hydroxy, C₁-C₄-alkyl-, C₁-C₆-alkoxy-, fluoro-C₁-C₃-alkyl-, hydroxy-C₁-C₃-alkyl-, C₃-C₈-cycloalkyl-, phenyl, monocyclic heterocyclyl- having 4 to 7 ring atoms or monocyclic heteroaryl- having 5 or 6 ring atoms,
and their polymorphs, enantiomers, diastereomers, racemates, tautomers, solvates, physiologically acceptable salts and solvates of these salts.

6. Compounds of the general formula (I) according to claim 1 in which
X represents an oxygen atom,
A represents a phenyl ring,
R^{1a} represents the radicals bicyclo[2.2.1]heptyl-, spiro[3.3]heptyl-, bicyclo[3.2.1]octyl-, spiro[3.4]octyl-, bicyclo[4.2.1]nonyl-, spiro[3.5]nonyl-, spiro[4.5]decyl- which contain one, two or three identical or different heteroatoms selected from the group consisting of oxygen, nitrogen and sulphur, and which may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl, phenyl-C₁-C₂-alkyl, phenoxy- and —C(═O)—R⁸,
or
represents the radicals perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl- which may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl, phenyl-C₁-C₂-alkyl, phenoxy- and —C(═O)—R⁸,
n represents 0 or 1,
R^{1b} represents fluorine, chlorine or cyano,
R² represents methyl,
R³ represents methyl or C₁-C₃-alkylamino-,
R⁴ and R⁵ independently of one another represent hydrogen, hydroxy, cyano, aminocarbonyl-, fluorine, chlorine, bromine,
or
represent C₁-C₄-alkyl-, C₁-C₄-alkoxy- which may optionally be mono- or polysubstituted by identical or different substituents from the group consisting of fluorine, amino, hydroxy, carboxy, C₁-C₃-alkoxy,
or
represent monocyclic heteroaryl- having 5 or 6 ring atoms which may optionally be mono- or polysubstituted by identical or different substituents from the group consisting of halogen, cyano, nitro, C₁-C₃-alkyl-, C₁-C₃-alkoxy-, fluoro-C₁-C₃-alkyl- and fluoro-C₁-C₃-alkoxy-,
or
represent monocyclic heterocyclyl- having 4 to 7 ring atoms which may optionally be mono- or polysubstituted by identical or different substituents from the group consisting of halogen, cyano, oxo, C₁-C₃-alkyl-, C₁-C₃-alkoxy-, fluoro-C₁-C₃-alkyl- and fluoro-C₁-C₃-alkoxy-,
or
represent phenyl which may optionally be mono- or polysubstituted by identical or different substituents from the group consisting of halogen, cyano, C₁-C₃-alkyl-, C₁-C₃-alkoxy-, fluoro-C₁-C₃-alkyl- and fluoro-C₁-C₃-alkoxy-,
R⁸ represents C₁-C₄-alkyl or C₁-C₄-alkoxy-,
and their polymorphs, enantiomers, diastereomers, racemates, tautomers, solvates, physiologically acceptable salts and solvates of these salts.

7. Compounds of the general formula (I) according to claim 1 in which
X represents an oxygen atom,
A represents a phenyl ring,
R^{1a} represents the radicals bicyclo[2.2.1]heptyl-, spiro[3.3]heptyl-, bicyclo[3.2.1]octyl-, spiro[3.4]octyl-, bicyclo[4.2.1]nonyl-, spiro[3.5]nonyl-, spiro[4.5]decyl- which contain one, two or three identical or different heteroatoms selected from the group consisting of oxygen, nitrogen and sulphur and which may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₈-cycloalkyl, monocyclic heterocyclyl- having 3 to 8 ring members, phenyl, halophenyl-, phenyl-C₁-C₂-alkyl-, phenoxy- and —C(═O)—R⁸,
or
represents the radicals perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl- which may optionally be mono- or disubstituted by identical or different radicals from the group consisting of oxo, halogen, cyano, hydroxy, C₁-C₄-alkyl, fluoro-C₁-C₆-alkyl-, C₁-C₄-alkoxy, fluoro-C₁-C₄-alkoxy, C₃-C₈-cycloalkyl, monocyclic heterocyclyl- having 3 to 8 ring members, phenyl, halophenyl-, phenyl-C₁-C₂-alkyl-, phenoxy- and —C(═O)—R⁸,
n represents 0 or 1,
R^{1b} represents fluorine, chlorine or cyano,
R² represents methyl,
R³ represents methyl or C₁-C₃-alkylamino-,
R⁴ represents hydrogen or C₁-C₃-alkoxy-,
R⁵ represents hydrogen, C₁-C₃-alkoxy or fluoro-C₁-C₃-alkoxy-,
or
represents a monocyclic heteroaryl radical having 5 or 6 ring atoms which may be mono- or disubstituted by C₁-C₃-alkyl, C₁-C₃-alkoxy- or halogen,
R⁸ represents C₁-C₄-alkyl or C₁-C₄-alkoxy-,
and their polymorphs, enantiomers, diastereomers, racemates, tautomers, solvates, physiologically acceptable salts and solvates of these salts.

8. Compounds of the general formula (I) according to claim 1 in which
X represents an oxygen atom,
A represents a phenyl ring,
R^{1a} represents the radicals 2-azabicyclo[2.2.1]heptyl-, 2,5-diazabicyclo[2.2.1]heptyl-, 2-oxa-5-azabicyclo[2.2.1]heptyl-, 2-azaspiro[3.3]heptyl-, 1-thia-6-azaspiro[3.3]heptyl-, 2-thia-6-azaspiro[3.3]heptyl-, 2-oxa-6-azaspiro[3.3]heptyl-, 2,6-diazaspiro[3.3]heptyl-, 8-oxa-3-azabicyclo[3.2.1]octyl-, 8-azabicyclo[3.2.1]octyl-, 2-oxa-6-azaspiro[3.4]octyl-, 3,9-diazabicyclo[4.2.1]nonyl-, 2-oxa-6-azaspiro[3.5]nonyl-, 2-oxa-7-azaspiro[3.5]nonyl-, 8-azaspiro[4.5]decyl-, 2,8-diazaspiro[4.5]decyl-, 3-oxa-1,8-diazaspiro[4.5]decyl-, perhydrofuro[3,2-c]pyridinyl-, perhydropyrrolo[1,2-a]pyrazinyl-, perhydropyrrolo[3,4-c]pyrrolyl-, quinolinyl-, isoquinolinyl-, indolyl-, 2,3-dihydro-1,4-benzodioxinyl-, 1,3-benzodioxolyl-, 2,3-dihydro-1-benzofuranyl-, where the radicals mentioned may optionally be mono- or disubstituted by identical or different radicals from the group consisting of oxo, halogen, cyano, hydroxy, C₁-C₄-alkyl, fluoro-C₁-C₄-alkyl-, C₁-C₄-alkoxy, fluoro-C₁-C₄-alkoxy, C₃-C₆-cycloalkyl-, monocyclic heterocyclyl- having 3 to 8 ring members, phenyl, halophenyl-, phenyl-C₁-C₂-alkyl-, phenoxy- and —C(═O)—R⁸,
n represents 0 or 1,
R^{1b} represents fluorine,
R² represents methyl,
R³ represents methyl or C₁-C₃-alkylamino-,
R⁴ represents hydrogen or C₁-C₃-alkoxy-,
R⁵ represents hydrogen, C₁-C₃-alkoxy- or fluoro-C₁-C₃-alkoxy-,
or
represents a monocyclic heteroaryl radical having 5 ring atoms which contains at least one nitrogen atom through which it is attached to the remainder of the molecule, and which may be mono- or disubstituted by C₁-C₃-alkyl or halogen,
R⁸ represents C₁-C₄-alkyl or C₁-C₄-alkoxy-,
and their polymorphs, enantiomers, diastereomers, racemates, tautomers, solvates, physiologically acceptable salts and solvates of these salts.

9. Compounds of the general formula (I) according to claim 1 in which
X represents an oxygen atom,
A represents a phenyl ring,
R^{1a} represents the radicals 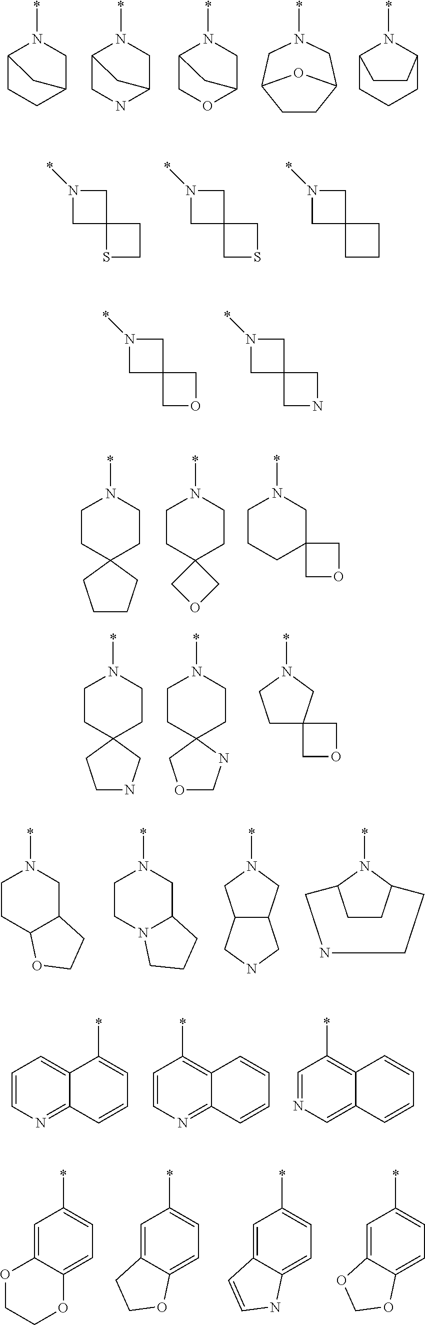
where “*” denotes the point of attachment to the remainder of the molecule,
and the radicals may optionally be mono- or disubstituted by identical or different substituents from the group consisting of oxo, halogen, cyano, hydroxy, C₁-C₄-alkyl, fluoro-C₁-C₄-alkyl-, C₁-C₄-alkoxy-, fluoro-C₁-C₄-alkoxy-, C₃-C₈-cycloalkyl-, monocyclic heterocyclyl- having 3 to 8 ring members, phenyl, halophenyl-, phenyl-C₁-C₂-alkyl-, phenoxy- and —C(═O)—R⁸,
n represents 0 or 1,
R^{1b} represents fluorine,
R² represents methyl,
R³ represents C₁-C₃-alkylamino-,
R⁴ represents hydrogen or methoxy-,
R⁵ represents methoxy-, trifluoromethoxy- or 3,5-dimethylpyrazol-1-yl,
R⁸ represents C₁-C₄-alkyl or C₁-C₄-alkoxy,
and their polymorphs, enantiomers, diastereomers, racemates, tautomers, solvates, physiologically acceptable salts and solvates of these salts.

10. Compounds of the general formula (I) according to claim 1 in which
X represents an oxygen atom,
A represents a phenyl ring,
R^{1a} represents the radicals 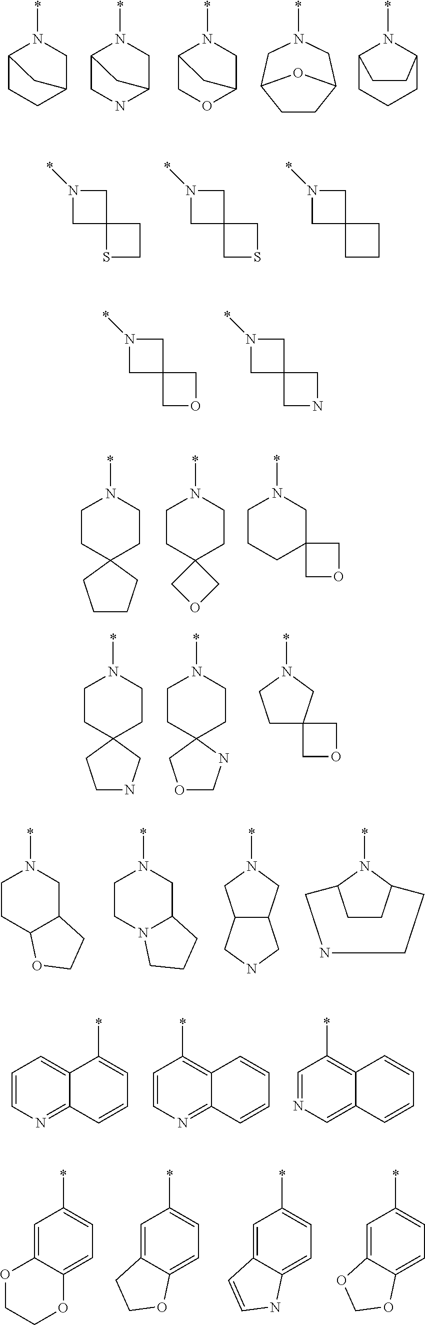
where “*” denotes the point of attachment to the remainder of the molecule and the radicals may optionally be mono- or disubstituted by identical or different substituents from the group consisting of oxo, fluorine, chlorine, bromine, cyano, hydroxy, methyl, ethyl, methoxy-, ethoxy-, benzyl-, phenyl-, phenoxy- and —C(═O)—R⁸,
n represents 0 or 1,
R^{1b} represents fluorine,
R² represents methyl,
R³ represents C₁-C₃-alkylamino-,
R⁴ represents hydrogen or methoxy-,
R⁵ represents methoxy-, trifluoromethoxy- or 3,5-dimethylpyrazol-1-yl,
R⁸ represents methyl or tert-butoxy-,
and their polymorphs, enantiomers, diastereomers, racemates, tautomers, solvates, physiologically acceptable salts and solvates of these salts.

11. Compounds of the general formula (I) according to claim 1 in which
X represents an oxygen atom,
A represents a phenyl ring,
R^{1a} represents the radicals 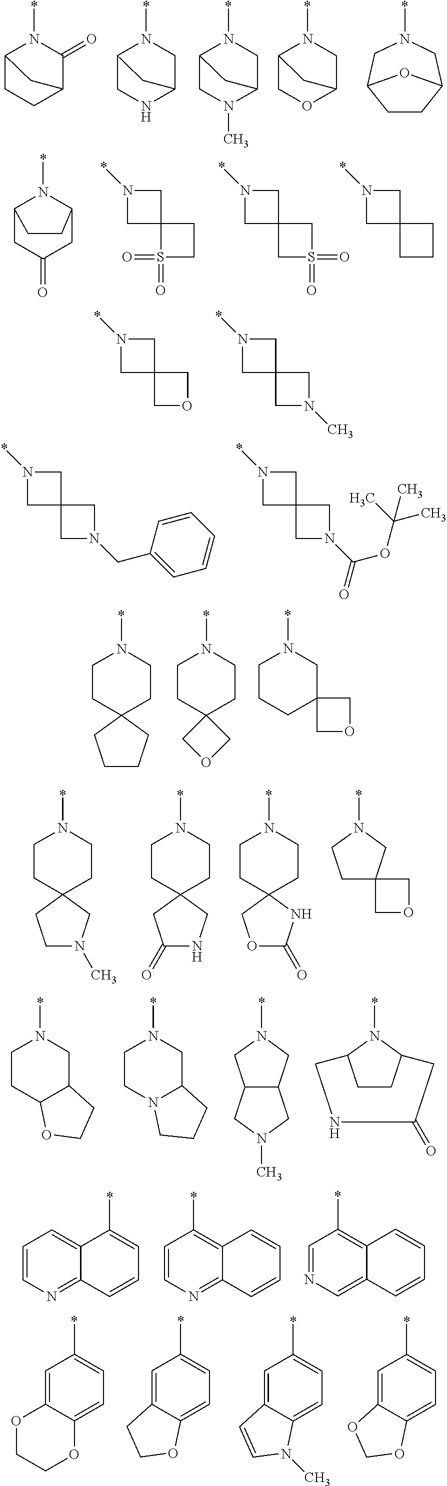
where “*” denotes the point of attachment to the remainder of the molecule,
n represents 0 or 1,
R^{1b} represents fluorine,
R² represents methyl-,
R³ represents methylamino-,
R⁴ represents hydrogen or methoxy-,
R⁵ represents methoxy-, trifluoromethoxy- or 3,5-dimethylpyrazol-1-yl, and their polymorphs, enantiomers, diastereomers, racemates, tautomers, solvates, physiologically acceptable salts and solvates of these salts.

12. Compounds of the general formula (I) according to claim 1:
[1S-(1R*,4S*)]-7,8-dimethoxy-N,4-dimethyl-1-[4-(3-oxo-2-azabicyclo[2.2.1]hept-2-yl)phenyl]-4,5-dihydro-3H-2,3-benzodiazepine-3-carboxamide;
{1S-[1R*,2(S*),4S*]}-7,8-dimethoxy-N,4-dimethyl-1-[4-(-3-oxo-2-azabicyclo[2.2.1]hept-2-yl)phenyl]-4,5-dihydro-3H-2,3-benzodiazepine-3-carboxamide;
(4S)-1-[4-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(4R)-1-[4-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(±)-1-[4-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(8-oxa-3-azabicyclo[3.2.1]oct-3-yl)phenyl]-4,5-dihydro-3H-2,3-benzodiazepine-3-carboxamide;
(4R)-7,8-dimethoxy-N,4-dimethyl-1-[4-(8-oxa-3-azabicyclo[3.2.1]oct-3-yl)phenyl]-4,5-dihydro-3H-2,3-benzodiazepine-3-carboxamide;
(4S)-7,8-dimethoxy-N,4-dimethyl-1-[4-(8-oxa-3-azabicyclo[3.2.1]oct-3-yl)phenyl]-4,5-dihydro-3H-2,3-benzodiazepine-3-carboxamide;
(±)-7,8-dimethoxy-N,4-dimethyl-1-[4-(2-oxa-6-azaspiro[3.3]hept-6-yl)phenyl]-4,5-dihydro-3H-2,3-benzodiazepine-3-carboxamide;
(4S)-7,8-dimethoxy-N,4-dimethyl-1-[4-(2-oxa-6-azaspiro[3.3]hept-6-yl)phenyl]-4,5-dihydro-3H-2,3-benzodiazepine-3-carboxamide;
(4R)-7,8-dimethoxy-N,4-dimethyl-1-[4-(2-oxa-6-azaspiro[3.3]hept-6-yl)phenyl]-4,5-dihydro-3H-2,3-benzodiazepine-3-carboxamide;
(±)-7,8-dimethoxy-N,4-dimethyl-1-[4-(3-oxo-8-azabicyclo[3.2.1]oct-8-yl)phenyl]-4,5-dihydro-3H-2,3-benzodiazepine-3-carboxamide;
(4R)-7,8-dimethoxy-N,4-dimethyl-1-[4-(3-oxo-8-azabicyclo[3.2.1]oct-8-yl)phenyl]-4,5-dihydro-3H-2,3-benzodiazepine-3-carboxamide;
(4S)-7,8-dimethoxy-N,4-dimethyl-1-[4-(3-oxo-8-azabicyclo[3.2.1]oct-8-yl)phenyl]-4,5-dihydro-3H-2,3-benzodiazepine-3-carboxamide;
tert-butyl 6-{4-[(±)-7,8-dimethoxy-4-methyl-3-(methylcarbamoyl)-4,5-dihydro-3H-2,3-benzodiazepine-1-yl]phenyl}-2,6-diazaspiro[3.3]heptane-2-carboxylate;
tert-butyl 6-{4-[(4S)-7,8-dimethoxy-4-methy-3-(methylcarbamoyl)-4,5-dihydro-3H-2,3-benzodiazepine-1-yl]phenyl}-2,6-diazaspiro[3.3]heptane-2-carboxylate;
tert-butyl 6-{4-[(4R)-7,8-dimethoxy-4-methyl-3-(methylcarbamoyl)-4,5-dihydro-3H-2,3-benzodiazepine-1-yl]phenyl}-2,6-diazaspiro[3.3]heptane-2-carboxylate;
(±)-1-[4-(8-azaspiro[4.5]dec-8-yl)phenyl]-7,8-dimethoxy-N,4-dimethyl-4,5-dihydro-3H-2,3-benzodiazepine-3-carboxamide;
(4S)-1-[4-(8-azaspiro[4.5]dec-8-yl)phenyl]-7,8-dimethoxy-N,4-dimethyl-4,5-dihydro-3H-2,3-benzodiazepine-3-carboxamide;
(4R)-1-[4-(8-azaspiro[4.5]dec-8-yl)phenyl]-7,8-dimethoxy-N,4-dimethyl-4,5-dihydro-3H-2,3-benzodiazepine-3-carboxamide;
(±)-1-[4-(6-benzyl-2,6-diazaspiro[3.3]hept-2-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(2-oxa-6-azaspiro[3.5]non-6-yl)phenyl]-4,5-dihydro-3H-2,3-benzodiazepine-3-carboxamide;
(±)-7,8-dimethoxy-N,4-dimethyl-1-[4-(2-oxa-7-azaspiro[3.5]non-7-yl)phenyl]-4,5-dihydro-3H-2,3-benzodiazepine-3-carboxamide;
(±)-7,8-dimethoxy-N,4-dimethyl-1-[3-(8-oxa-3-azabicyclo[3.2.1]oct-3-yl)phenyl]-4,5-dihydro-3H-2,3-benzodiazepine-3-carboxamide;
(±)-1-[3-(1,1-dioxido-1-thia-6-azaspiro[3.3]hept-6-yl)phenyl]-7,8-dimethoxy-N,4-dimethyl-4,5-dihydro-3H-2,3-benzodiazepine-3-carboxamide;
(±)-1-[3-(1,1-dioxido-1-thia-6-azaspiro[3.3]hept-6-yl)-4-fluorophenyl]-7,8-dimethoxy-N,4-dimethyl-4,5-dihydro-3H-2,3-benzodiazepine-3-carboxamide;
(±)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide;
(4R)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide;
(4S)-8-(3,5-dimethyl-1H-pyrazol-1-yl)-1-[4-(1,1-dioxido-1-thia-6-azaspiro[3.3]hept-6-yl)phenyl]-N,4-dimethyl-4,5-dihydro-3H-2,3-benzodiazepine-3-carboxamide;
(±)-1-[4-(1,1-dioxido-1-thia-6-azaspiro[3.3]hept-6-yl)phenyl]-N,4-dimethyl-8-(trifluoromethoxy)-4,5-dihydro-3H-2,3-benzodiazepine-3-carboxamide;
(4R)-1-[4-(1,1-dioxido-1-thia-6-azaspiro[3.3]hept-6-yl)phenyl]-N,4-dimethyl-8-(trifluoromethoxy)-4,5-dihydro-3H-2,3-benzodiazepine-3-carboxamide;
(4S)-1-[4-(1,1-dioxido-1-thia-6-azaspiro[3.3]hept-6-yl)phenyl]-N,4-dimethyl-8-(trifluoromethoxy)-4,5-dihydro-3H-2,3-benzodiazepine-3-carboxamide;
[1S-(1R*,4R*)]-1-[4-(2,5-diazabicyclo[2.2.1]hept-2-yl)phenyl]-7,8-dimethoxy-4,5-dihydro-N,4-dimethyl-3H-2,3-benzodiazepine-3-carboxamide;
[1S-(1R*,4R*)]-1-[3-(2,5-diazabicyclo[2.2.1]hept-2-yl)-4-fluorophenyl]-7,8-dimethoxy-4,5-dihydro-N,4-dimethyl-3H-2,3-benzodiazepine-3-carboxamide;
(±)-1-[4-(2,3-dihydro-1,4-benzodioxin-6-yl)phenyl]-7,8-dimethoxy-N,4-dimethyl-4,5-dihydro-3H-2,3-benzodiazepine-3-carboxamide;
(±)-1-[4-(2,3-dihydro-1-benzofuran-5-yl)phenyl]-7,8-dimethoxy-N,4-dimethyl-4,5-dihydro-3H-2,3-benzodiazepine-3-carboxamide;
(±)-1-[4-(quinolin-5-yl)phenyl]-7,8-dimethoxy-N,4-dimethyl-4,5-dihydro-3H-2,3-benzodiazepine-3-carboxamide;
(±)-1-[4-(quinolin-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(1-methyl-1H-indol-5-yl)phenyl]-4,5-dihydro-3H-2,3-benzodiazepine-3-carboxamide;
(±)-1-[4-(isoquinolin-4-yl)phenyl]-7,8-dimethoxy-N,4-dimethyl-4,5-dihydro-3H-2,3-benzodiazepine-3-carboxamide;
(±)-1-[4-(1,3-benzodioxol-5-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(3-oxo-2,8-diazaspiro[4.5]dec-8-yl)phenyl]-4,5-dihydro-3H-2,3-benzodiazepine-3-carboxamide;
(±)-7,8-dimethoxy-N,4-dimethyl-1-[4-(2-oxa-6-azaspiro[3.4]oct-6-yl)phenyl]-4,5-dihydro-3H-2,3-benzodiazepine-3-carboxamide;
(±)-1-[4-(hexahydropyrrolo[1,2-a]pyrazin-2(1H)-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(2-methyl-2,8-diazaspiro[4.5]dec-8-yl)phenyl]-4,5-dihydro-3H-2,3-benzodiazepine-3-carboxamide;
(±)-7,8-dimethoxy-N,4-dimethyl-1-[4-(2-oxo-3-oxa-1,8-diazaspiro[4.5]dec-8-yl)phenyl]-4,5-dihydro-3H-2,3-benzodiazepine-3-carboxamide;
(±)-7,8-dimethoxy-N,4-dimethyl-1-{4-[(3aR,6aS)-5-methylhexahydropyrrolo[3,4-c]pyrrol-2(1H)-yl]phenyl}-4,5-dihydro-3H-2,3-benzodiazepine-3-carboxamide;
(±)-1-[4-(2,2-dioxido-2-thia-6-azaspiro[3.3]hept-6-yl)phenyl]-7,8-dimethoxy-N,4-dimethyl-4,5-dihydro-3H-2,3-benzodiazepine-3-carboxamide;
(4S)-7,8-dimethoxy-N,4-dimethyl-1-{4-[(1S,4S)-2-oxa-5-azabicyclo[2.2.1]hept-5-yl]phenyl}-4,5-dihydro-3H-2,3-benzodiazepine-3-carboxamide;
(±)-1-[4-(hexahydrofuro[3,2-c]pyridin-5(4H-yl)phenyl]-7,8-dimethoxy-N,4-dimethyl-4,5-dihydro-3H-2,3-benzodiazepine-3-carboxamide;
(±)-1-[4-(2-azaspiro[3.3]hept-2-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(6-methyl-2,6-diazaspiro[3.3]hept-2-yl)phenyl]-4,5-dihydro-3H-2,3-benzodiazepine-3-carboxamide;
(±)-7,8-dimethoxy-N,4-dimethyl-1-[4-(4-oxo-3,9-diazabicyclo[4.2.1]non-9-yl)phenyl]-4,5-dihydro-3H-2,3-benzodiazepine-3-carboxamide;
[1S-(1R*,4R*)]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide;
[1S-[1R*,2(S*),4R*]]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide;
[1S-[1R*,2(R*),4R*]]-7,8-dimethoxy-N,4-dimethyl-1-[4-(5-methyl-2,5-diazabicyclo[2.2.1]hept-2-yl)phenyl]-4,5-dihydro-3H-2,3-benzodiazepine-3-carboxamide;
[1S-(1R*,4R*)]-1-[4-fluoro-3-(5-methyl-2,5-diazabicyclo[2.2.1]hept-2-yl)phenyl]-7,8-dimethoxy-4,5-dihydro-N,4-dimethyl-3H-2,3-benzodiazepine-3-carboxamide;
[1S-(1R*,2(R*),4R*)]-1-[4-fluoro-3-(5-methyl-2,5-diazabicyclo[2.2.1]hept-2-yl)phenyl]-7,8-dimethoxy-4,5-dihydro-N,4-dimethyl-3H-2,3-benzodiazepine-3-carboxamide and
[1S-(1R*,2(S*),4R*)]-1-[4-fluoro-3-(5-methyl-2,5-diazabicyclo[2.2.1]hept-2-yl)phenyl]-7,8-dimethoxy-4,5-dihydro-N,4-dimethyl-3H-2,3-benzodiazepine-3-carboxamide.

13. A method for treating a subject suffering from any of hyperproliferative disorders, benign hyperplasias, inflammatory disorders, autoimmune disorders, sepsis, viral infections, vascular disorders, atherosclerotic disorders and neurodegenerative disorders comprising administering the compound of claim 1 to the subject in an effective amount thereby treating the disorder.

14. A method for treating a subject suffering from tumour disorders comprising administering the compound of claim 1 to the subject in an effective amount thereby treating the disorder.

15. A method for controlling male fertility in a subject comprising administering to the subject the Compound of claim 1 in a sufficient amount thereby controlling male fertility.

16. A method for treating a subject suffering from leukaemias, prostate carcinomas, mammary carcinomas, melanomas or multiple myelomas comprising administering the compound of claim 1 to the subject in an effective amount thereby treating the disorder.

17. (canceled)

18. (canceled)

19. A composition comprising the Compound of the formula (I) of claim 1 in combination with a further active compound.

20. Pharmaceutical formulation comprising a compound of the formula (I) of claim 1.
